(19) United States
(12) Patent Application Publication
(10) Pub. No.: US 2009/0246754 A1
(43) Pub. Date: Oct. 1, 2009

(54) OPTIMIZED PROBES AND PRIMERS AND METHODS OF USING SAME FOR THE DETECTION AND QUANTITATION OF BK VIRUS

(75) Inventors: Heather L.B. Kiefer, Westford, MA (US); James R. Hully, Salisbury, MA (US); William Lauzier, East Bridgewater, MA (US); Julian Chandler, Allston, MA (US); Damien Slater, Roslindale, MA (US)

Correspondence Address: Intelligent Medical Devices, Inc.;C/O Foley & Lardner LLP, 111 Huntington Avenue, 26th Floor, Boston, MA, 02199-7610, US

(73) Assignee: Intelligent MDx

(21) Appl. No.: 12/388,378

(22) Filed: Feb. 18, 2009

Related U.S. Application Data
(60) Provisional application No. 61/029,724, filed on Feb. 19, 2008.

(51) Int. Cl. C12Q 1/70; C07H 21/04

(52) U.S. Cl. 435/5; 536/24.32; 536/24.33

(57) ABSTRACT

Described herein are probes and primers for detecting and quantitating variant BK viral strains and methods and devices of identifying and using the described probes and primers.

### RELATED APPLICATION

This application claims the benefit of U.S. Provisional Patent Application No. 61/029,724, filed Feb. 19, 2008, the contents of which are incorporated by reference herein in its entirety.

### BACKGROUND OF THE INVENTION

BK virus (BKV) is a ubiquitous polyomavirus that infects approximately 80% of adults in the United States. Adults with primary BKV infections are typically asymptomatic or are minimally symptomatic with fever and non-specific upper respiratory infection symptoms. After primary infection, however, a latent infection may occur in renal epithelial cells and possibly other tissues such as the brain. When this happens in immunocompromised or immunosuppressed patients, BKV can reactivate and cause serious disease. BKV-associated nephropathy is thus a leading cause of renal dysfunction and transplant failure in kidney transplant patients. Accordingly, whenever an immunocompromised or immunosuppressed individual suffers renal dysfunction, a test should be performed to identify resurgence of latent BKV infection. When a diagnosis of BKV is made, reducing the dose of immunosuppressive therapy and monitoring viral load has been the standard of care.

Biopsy-based evidence of infection has been used for diagnosis because BKV is difficult to culture and can take weeks to grow. Nucleic acid diagnostic testing kits are available, but they cannot adequately identify the broad genetic diversity of target BKV pathogens. A rapid and accurate diagnostic test for the detection and quantitation of BKV, therefore, regardless of the specific infecting variant, would support effective treatments and control of infection.

### SUMMARY OF THE INVENTION

Disclosed herein are nucleic acid probes and primers for detecting and quantitating viral genetic material from BK virus, and methods and devices for using the probes and primers.

One embodiment is directed to n isolated polynucleotide comprising a nucleotide sequence selected from the group consisting of: SEQ ID NOS: 1-334.

One embodiment is directed to a PCR primer pair for amplifying BK virus or one or more variants thereof, comprising a pair of sequences selected from the group consisting of: (1) SEQ ID NOS: 1 and 3; (2) SEQ ID NOS: 6 and 3; (3) SEQ ID NOS: 7 and 3; (4) SEQ ID NOS: 8 and 3; (5) SEQ ID NOS: 9 and 3; (6) SEQ ID NOS: 10 and 12; (7) SEQ ID NOS: 13 and 12; (8) SEQ ID NOS: 15 and 17; (9) SEQ ID NOS: 15 and 19; (10) SEQ ID NOS: 20 and 21; (11) SEQ ID NOS: 15 and 22; (12) SEQ ID NOS: 23 and 25; (13) SEQ ID NOS: 26 and 25; (14) SEQ ID NOS: 28 and 29; (15) SEQ ID NOS: 15 and 30; (16) SEQ ID NOS: 28 and 31; (17) SEQ ID NOS: 15 and 32; (18) SEQ ID NOS: 20 and 32; (19) SEQ ID NOS: 10 and 33; (20) SEQ ID NOS: 10 and 34; (21) SEQ ID NOS: 23 and 35; (22) SEQ ID NOS: 28 and 36; (23) SEQ ID NOS: 37 and 39; (24) SEQ ID NOS: 37 and 36; (25) SEQ ID NOS: 28 and 33; (26) SEQ ID NOS: 13 and 32; (27) SEQ ID NOS: 42 and 43; (28) SEQ ID NOS: 28 and 44; (29) SEQ ID NOS: 10 and 45; (30) SEQ ID NOS: 15 and 46; (31) SEQ ID NOS: 10 and 47; (32) SEQ ID NOS: 20 and 48; (33) SEQ ID NOS: 10 and 49; (34) SEQ ID NOS: 15 and 50; (35) SEQ ID NOS: 10 and 51; (36) SEQ ID NOS: 52 and 54; (37) SEQ ID NOS: 20 and 47; (38) SEQ ID NOS: 10 and 49; (39) SEQ ID NOS: 55 and 57; (40) SEQ ID NOS: 58 and 19; (41) SEQ ID NOS: 60 and 22; (42) SEQ ID NOS: 55 and 62; (43) SEQ ID NOS: 58 and 34; (44) SEQ ID NOS: 26 and 66; (45) SEQ ID NOS: 37 and 67; (46) SEQ ID NOS: 68 and 50; (47) SEQ ID NOS: 20 and 70; (48) SEQ ID NOS: 10 and 72; (49) SEQ ID NOS: 55 and 73; (50) SEQ ID NOS: 55 and 74; (51) SEQ ID NOS: 37 and 75; (52) SEQ ID NOS: 76 and 25; (53) SEQ ID NOS: 55 and 78; (54) SEQ ID NOS: 26 and 79; (55) SEQ ID NOS: 80 and 82; (56) SEQ ID NOS: 58 and 84; (57) SEQ ID NOS: 76 and 29; (58) SEQ ID NOS: 15 and 21; (59) SEQ ID NOS: 23 and 35; (60) SEQ ID NOS: 87 and 25; (61) SEQ ID NOS: 88 and 25; (62) SEQ ID NOS: 10 and 89; (63) SEQ ID NOS: 10 and 90; (64) SEQ ID NOS: 20 and 91; (65) SEQ ID NOS: 55 and 78; (66) SEQ ID NOS: 92 and 94; (67) SEQ ID NOS: 95 and 57; (68) SEQ ID NOS: 97 and 94; (69) SEQ ID NOS: 100 and 94; (70) SEQ ID NOS: 97 and 103; (71) SEQ ID NOS: 95 and 12; (72) SEQ ID NOS: 104 and 94; (73) SEQ ID NOS: 97 and 105; (74) SEQ ID NOS: 97 and 21; (5) SEQ ID NOS: 106 and 94; (76) SEQ ID NOS: 107 and 103; (77) SEQ ID NOS: 109 and 103; (78) SEQ ID NOS: 111 and 112; (79) SEQ ID NOS: 113 and 94; (80) SEQ ID NOS: 114 and 103; (81) SEQ ID NOS: 115 and 116; (82) SEQ ID NOS: 117 and 94; (83) SEQ ID NOS: 109 and 105; (84) SEQ ID NOS: 18 and 94; (85) SEQ ID NOS: 119 and 103; (86) SEQ ID NOS: 120 and 103; (87) SEQ ID NOS: 121 and 103; (88) SEQ ID NOS: 123 and 105; (89) SEQ ID NOS: 125 and 103 (90) SEQ ID NOS: 126 and 12; (91) SEQ ID NOS: 128 and 62; (92) SEQ ID NOS: 129 and 105; (93) SEQ ID NOS: 130 and 12; (94) SEQ ID NOS: 131 and 94; (95) SEQ ID NOS: 132 and 94; (96) SEQ ID NOS: 107 and 12; (97) SEQ ID NOS: 97 and 57; and (98) SEQ ID NOS: 98 ad 57.

One embodiment is directed to a PCR primer pair for amplifying BK virus or one or more variants thereof, comprising a pair of sequences selected from the group consisting of: (1) SEQ ID NOS: 147 and 234; (2) SEQ ID NOS: 146 and 217; (3) SEQ ID NOS: 148 and 225; (4) SEQ ID NOS: 172 and 250 (5) SEQ ID NOS: 138 and 216; (6) SEQ ID NOS: 139 and 221; (7) SEQ ID NOS: 150 and 227; (8) SEQ ID NOS: 145 and 233; (9) SEQ ID NOS: 142 and 226; (10) SEQ ID NOS: 170 and 248; (1) SEQ ID NOS: 151 and 230; (12) SEQ ID NOS: 158 and 241; (13) SEQ ID NOS: 150 and 238; (14) SEQ ID NOS: 147 and 218; (15) SEQ ID NOS: 146 and 228; (16) SEQ ID NOS: 136and 237; (17) SEQ ID NOS: 172 and 249; (18) SEQ ID NOS: 138 and 223; (19) SEQ ID NOS: 143 and 219 (20) SEQ ID NOS: 157 and 243; (21) SEQ ID NOS: 139 and 223; (22) SEQ ID NOS: 150 and 235; (23) SEQ ID NOS: 150 and 232; (24) SEQ ID NOS: 147 and 233; (25) SEQ ID NOS: 146 and 237; (26) SEQ ID NOS: 142 and 223; (27) SEQ ID NOS: 148 and 215; (28) SEQ ID NOS: 139 and 230 (29) SEQ ID NOS: 162 and 242; (30) SEQ ID NOS: 137 and 215; (31) SEQ ID NOS: 140 and 222; (32) SEQ ID NOS: 166 and 241; (33) SEQ ID NOS: 156 and 234; (34) SEQ ID NOS: 143 and 221; (35) SEQ ID NOS: 165 and 242; (36) SEQ ID NOS: 149 and 230; (37) SEQ ID NOS: 141 and 223; (38) SEQ ID NOS: 147 and 236 (39) SEQ ID NOS: 146 and 229 (40) SEQ ID NOS: 168 and 251; (41) SEQ ID NOS: 171 and 248; (42) SEQ ID NOS: 138 and 220; (43) SEQ ID NOS: 160 and 241; (44) SEQ ID NOS: 164 and 252; (45) SEQ ID NOS: 139 and 231 (46) SEQ ID NOS: 150 and 223; (47) SEQ ID NOS: 144 and 233; (48) SEQ ID NOS: 170 and 252; (49) SEQ ID NOS: 163 and 242; (50) SEQ ID NOS: 150 and 228; (51) SEQ ID NOS: 147 and 226; (52) SEQ ID NOS: 140 and 217; (53) SEQ ID NOS: 172 and 247; (54) SEQ ID NOS: 143 and 230; (55) SEQ ID NOS: 139 and 224; (56) SEQ ID NOS: 150 and 235; (57) SEQ ID NOS: 184 and 253; (58) SEQ ID NOS: 168 and 255; (59) SEQ ID NOS: 175 and 255 (60) SEQ ID NOS: 173 and 253; (61) SEQ ID NOS: 164 and 255; (62) SEQ ID NOS: 177 and 253; (63) SEQ ID NOS: 169 and 253; (64) SEQ ID NOS: 183 and 255; (65) SEQ ID NOS: 172 and 245, (66) SEQ ID NOS: 166 and 254; (67) SEQ ID NOS: 178 and 254; (68) SEQ ID NOS: 165 and 255 (69) SEQ ID NOS: 174 and 253; (70) SEQ ID NOS: 171 and 254; (71) SEQ ID NOS: 160 and 255; (72) SEQ ID NOS: 172 and 246; (73) SEQ ID NOS: 175 and 254; (74) SEQ ID NOS: 161 and 253; (75) SEQ ID NOS: 159 and 255; (76) SEQ ID NOS: 167 and 253; (77) SEQ ID NOS: 183 and 254; (78) SEQ ID NOS: 181 and 255; (79) SEQ ID NOS: 150 and 240 (80) SEQ ID NOS: 179 and 253; (81) SEQ ID NOS: 172 and 254; (82) SEQ ID NOS: 176 and 253; (83) SEQ ID NOS: 183 and 253; (84) SEQ ID NOS: 166 and 255; (85) SEQ ID NOS: 154 and 239; (86) SEQ ID NOS: 178 and 253; (87) SEQ ID NOS: 153 and 239; (88) SEQ ID NOS: 152 and 239; (89) SEQ ID NOS: 185 and 254; (90) SEQ ID NOS: 171 and 255; (91) SEQ ID NOS: 155 and 239; (92) SEQ ID NOS: 182 and 255; (93) SEQ ID NOS:180 and 254; (94) SEQ ID NOS: 172 and 244; and (95) SEQ ID NOS: 183 and 253.

One embodiment is directed to a PCR primer pair for amplifying BK virus or one or more variants thereof, comprising a pair of sequences selected from the group consisting of: (1) SEQ ID NOS: 256 and 292; (2) SEQ ID NOS: 257 and 292; (3) SEQ ID NOS: 257 and 293; (4) SEQ ID NOS: 258 and 292; (5) SEQ ID NOS: 259 and 292; (6) SEQ ID NOS: 260 and 292; (7) SEQ ID NOS: 261 and 292; (8) SEQ ID NOS: 262 and 292; (9) SEQ ID NOS: 263 and 293; (10) SEQ ID NOS: 264 and 294; (11) SEQ ID NOS: 264 and 295; (12) SEQ ID NOS: 264 and 296; (13) SEQ ID NOS: 264 and 297; (14) SEQ ID NOS: 264 and 298; (15) SEQ ID NOS: 264 and 299; (16) SEQ ID NOS: 264 and 300; (17) SEQ ID NOS: 265 and 294; (18) SEQ ID NOS: 265 and 301; (19) SEQ ID NOS:265 and 302; (20) SEQ ID NOS: 265 and 303; (21) SEQ ID NOS: 265 and 304; (22) SEQ ID NOS: 265 and 305; (23) SEQ ID NOS: 265 and 299; (24) SEQ ID NOS: 265 and 306; (25) SEQ ID NOS: 265 and 307; (26) SEQ ID NOS: 266 and 294; (27) SEQ ID NOS: 266 and 301; (28) SEQ ID NOS: 266 and 296; (29) SEQ ID NOS: 266 and 303; (30) SEQ ID NOS: 266 and 298; (31) SEQ ID NOS: 266 and 308; (32) SEQ ID NOS: 266 and 309; (33) SEQ ID NOS: 266 and 300; (34) SEQ ID NOS: 267 and 294; (35) SEQ ID NOS: 267 and 301; (36) SEQ ID NOS: 267 and 296; (37) SEQ ID NOS: 267 and 303; (38) SEQ ID NOS: 267 and 298; (39) SEQ ID NOS: 267 and 305; (40) SEQ ID NOS: 267 and 299; (41) SEQ ID NOS: 267 and 310; (42) SEQ ID NOS: 267 and 300; (43) SEQ ID NOS: 268 and 308; (44) SEQ ID NOS: 268 and 299; (45) SEQ ID NOS: 268 and 310; (46) SEQ ID NOS: 268 and 300; (47) SEQ ID NOS: 269 and 311; (48) SEQ ID NOS: 269 and 312; (49) SEQ ID NOS: 269 and 313; (50) SEQ ID NOS: 269 and 314; (51) SEQ ID NOS: 269 and 315; (52) SEQ ID NOS: 269 and 316; (53) SEQ ID NOS: 269 and 306; (54) SEQ ID NOS: 270 and 294; (55) SEQ ID NOS: 270 and 317; (56) SEQ ID NOS:270 and 303; (57) SEQ ID NOS: 270 and 298; (58) SEQ ID NOS: 270 and 308; (59) SEQ ID NOS: 270 and 309, (60) SEQ ID NOS: 270 and 310; (61) SEQ ID NOS: 270 and 300; (62) SEQ ID NOS:271 and 294; (63) SEQ ID NOS: 271 and 301; (64) SEQ ID NOS: 271 and 296; (65) SEQ ID NOS: 271 and 303; (66) SEQ ID NOS: 271 and 298; (67) SEQ ID NOS: 271 and 305; (68) SEQ ID NOS: 271 and 318; (69) SEQ ID NOS: 271 and 309; (70) SEQ ID NOS: 271 and 319; (71) SEQ ID NOS: 271 and 300; (72) SEQ ID NOS: 272 and 320; (73) SEQ ID NOS: 272 and 310; (74) SEQ ID NOS: 272 and 307; (75) SEQ ID NOS: 272 and 321; (76) SEQ ID NOS: 272 and 299; (77) SEQ ID NOS: 273 and 322; (78) SEQ ID NOS: 273 and 323; (79) SEQ ID NOS: 273 and 324, (80) SEQ ID NOS: 274 and 325; (81) SEQ ID NOS: 274 and 326; (82) SEQ ID NOS: 274 and 327; (83) SEQ ID NOS: 274 and 328; (84) SEQ ID NOS: 275 and 325; (85) SEQ ID NOS: 275 and 323; (86) SEQ ID NOS: 275 and 327; (87) SEQ ID NOS: 276 and 323; (88) SEQ ID NOS: 276 and 326; (89) SEQ ID NOS: 277 and 325; (90) SEQ ID NOS: 277 and 326; (91) SEQ ID NOS: 277 and 327; (92) SEQ ID NOS: 278 and 325; (93) SEQ ID NOS: 278 and 323; (94) SEQ ID NOS: 278 and 326; (95) SEQ ID NOS: 278 and 327; (96) SEQ ID NOS: 279 and 325; (97) SEQ ID NOS: 279 and 326; (98) SEQ ID NOS: 279 and 327; (99) SEQ ID NOS: 279 and 328; (100) SEQ ID NOS: 280 and 325; (101) SEQ ID NOS: 280 and 326; (102) SEQ ID NOS: 280 and 327; (103) SEQ ID NOS: 280 and 328; (104) SEQ ID NOS: 281 and 325; (105) SEQ ID NOS: 281 and 323; (106) SEQ ID NOS: 281 and 327; (107) SEQ ID NOS: 281 and 328; (108) SEQ ID NOS: 329 and 331; (109) SEQ ID NOS: 330 and 321; and (110) SEQ ID NOS: 329 and 331.

5. A polynucleotide probe for binding to BK virus DNA, comprising a nucleotide sequence selected from the group consisting of, SEQ ID NOS: 2, 4, 5, 11, 14, 16, 18, 24, 27, 38, 40, 41, 53, 56, 59, 61, 63, 64, 65, 69, 71, 77, 81, 83, 85, 86, 93, 96, 99, 101, 102, 108, 11, 122, 124, 127, 133-134, 186-214, 282-291, 332-333. In a particular embodiment, the probe does not cross react with an identified nucleic acid sequence of a polyoma virus with high efficiency. In a particular embodiment, the probe is labeled for quantitating BK virus. In a particular embodiment, the probe is labeled for detecting BK virus In a particular embodiment, the probe comprises a detectable moiety selected from the group consisting of: a fluorescent label, a chemiluminescent label, a radioactive label, a quenching molecule, biotin and gold.

One embodiment is directed to a method for detecting BK virus in a sample, comprising: (1) contacting at least one forward and reverse primer pair listed in Tables 2, 3 or 4 to a sample; (2) conducting a polymerase chain reaction; and (3) detecting and/or quantitating the generation of an amplified PCR product, wherein the generation of an amplified PCR product indicates the presence of BK virus in the sample. In a particular embodiment, the forward and reverse primer pair are selected from the group consisting of: (1) SEQ ID NOS: 1 and 3; (2) SEQ ID NOS: 6 and 3; (3) SEQ ID NOS: 7 and 3; (4) SEQ ID NOS: 8 and 3; (5) SEQ ID NOS: 9 and 3; (6) SEQ ID NOS: 10 and 12; (7) SEQ ID NOS: 13 and 12; (8) SEQ ID NOS: 15 and 17; (9) SEQ ID NOS: 15 and 19; (10) SEQ ID NOS: 20 and 21; (11) SEQ ID NOS: 15 and 22; (12) SEQ ID NOS: 23 and 25; (13) SEQ ID NOS: 26 and 25; (14) SEQ ID NOS: 28 and 29; (15) SEQ ID NOS: 15 and 30; (16) SEQ ID NOS: 28 and 31; (17) SEQ ID NOS: 15 and 32; (18) SEQ ID NOS: 20 and 32; (19) SEQ ID NOS: 10 and 33; (20) SEQ ID NOS: 10 and 34; (21) SEQ ID NOS: 23 and 35; (22) SEQ ID NOS: 28 and 36; (23) SEQ ID NOS: 37 and 39; (24) SEQ ID NOS: 37 and 36; (25) SEQ ID NOS: 28 and 33; (26) SEQ ID NOS: 13 and 32; (27) SEQ ID NOS: 42 and 43; (28) SEQ ID NOS: 28 and 44; (29) SEQ ID NOS: 10 and 45; (30) SEQ ID NOS: 15 and 46; (31) SEQ ID NOS: 10 and 47; (32) SEQ ID NOS: 20 and 48; (33) SEQ ID NOS: 10 and 49; (34) SEQ ID NOS: 15 and 50; (35) SEQ ID NOS: 10 and 51; (36) SEQ ID NOS: 52 and 54; (37) SEQ ID NOS: 20 and 47; (38) SEQ ID NOS: 10 and 49; (39) SEQ ID NOS: 55 and 57; (40) SEQ ID NOS: 58 and 19; (41) SEQ ID NOS: 60 and 22; (42) SEQ ID NOS: 55 and 62; (43) SEQ ID NOS: 58 and 34; (44) SEQ ID NOS: 26 and 66; (45) SEQ ID NOS: 37 and 67; (46) SEQ ID NOS: 68 and 50; (47) SEQ ID NOS: 20 and 70; (48) SEQ ID NOS: 10 and 72; (49) SEQ ID NOS: 55 and 73; (50) SEQ ID NOS: 55 and 74; (51) SEQ ID NOS: 37 and 75; (52) SEQ ID NOS: 76 and 25; (53) SEQ ID NOS: 55 and 78; (54) SEQ ID NOS: 26 and 79; (55) SEQ ID NOS: 80 and 82; (56) SEQ ID NOS: 58 and 84; (57) SEQ ID NOS: 76 and 29; (58) SEQ ID NOS: 15 and 21; (59) SEQ ID NOS: 23 and 35; (60) SEQ ID NOS: 87 and 25; (61) SEQ ID NOS: 88 and 25; (62) SEQ ID NOS: 10 and 89; (63) SEQ ID NOS: 10 and 90; (64) SEQ ID NOS: 20 and 91; (65) SEQ ID NOS: 55 and 78; (66) SEQ ID ID NOS: 92 and 94; (67) SEQ ID NOS: 95 and 57; (68) SEQ ID NOS: 97 and 94; (69) SEQ ID NOS: 100 and 94; (70) SEQ ID NOS: 97 and 103; (71) SEQ ID NOS: 95 and 12; (72) SEQ ID NOS: 104 and 94; (73) SEQ ID NOS: 97 and 105; (74) SEQ ID NOS: 97 and 21; (75) SEQ ID NOS: 106 and 94; (76) SEQ ID NOS: 107 and 103; (77) SEQ ID NOS: 109 and 103 (78) SEQ ID NOS: 111 and 112; (79) SEQ ID NOS: 113 and 94; (80) SEQ ID NOS: 114 and 1037 (81) SEQ ID NOS: 115 and 116; (82) SEQ ID NOS: 117 and 94; (83) SEQ ID NOS: 109 and 105; (84) SEQ ID NOS: 118 and 94; (85) SEQ ID NOS: 119 and 103; (86) SEQ ID NOS: 120 and 103; (87) SEQ ID NOS: 121 and 103, (88) SEQ ID NOS: 123 and 105; (89) SEQ ID NOS: 125 and 103; (90) SEQ ID NOS: 126 and 12; (91) SEQ ID NOS: 128 and 62; (92) SEQ ID NOS: 129 and 105; (93) SEQ ID NOS: 130 and 12; (94) SEQ ID NOS: 131 and 94; (95) SEQ ID NOS: 132 and 94; (96) SEQ ID NOS: 107 and 12; (97) SEQ ID NOS: 97 and 57; and (98) SEQ ID NOS: 98 and 57. In a particular embodiment, the forward and reverse primer pair are selected from the group consisting of: (1) SEQ ID NOS: 147 and 234; (2) SEQ ID NOS: 146 and 217; (3) SEQ ID NOS: 148 and 225; (4) SEQ ID NOS: 172 and 250; (5) SEQ ID NOS: 138 and 216; (6) SEQ ID NOS: 139 and 221; (7) SEQ ID NOS: 150 and 227; (8) SEQ ID NOS: 145 and 233; (9) SEQ 11) NOS: 142 and 226; (10) SEQ ID NOS: 170 and 248; (11) SEQ ID NOS: 151 and 230: (12) SEQ ID NOS: 158 and 241; (13) SEQ ID NOS: 150 and 238; (14) SEQ ID NOS: 147 and 218; (15) SEQ ID NOS: 146 and 228; (16) SEQ ID NOS: 136 and 237; (17) SEQ ID NOS: 172 and 249; (18) SEQ ID NOS: 138 and 223; (9) SEQ ID NOS: 143 and 219; (20) SEQ ID NOS: 157 and 243; (21) SEQ ID NOS: 139 and 223; (22) SEQ ID NOS: 150 and 235; (23) SEQ ID NOS: 150 and 232 (24) SEQ ID NOS: 147 and 233; (25) SEQ ID NOS: 146 and 237; (26) SEQ ID NOS: 142 and 223; (27) SEQ ID NOS: 148 and 215; (28) SEQ ID NOS: 139 and 230; (29) SEQ ID NOS: 162 and 242; (30) SEQ ID NOS: 137 and 215; (31) SEQ ID NOS: 140 and 222; (32) SEQ ID NOS: 166 and 241; (33) SEQ ID NOS: 156 and 234; (34) SEQ ID NOS: 143 and 221; (35) SEQ ID NOS: 165 and 242; (36) SEQ ID NOS: 149 and 230; (37) SEQ ID NOS: 141 and 223; (38) SEQ ID NOS: 147 and 236; (39) SEQ ID NOS: 146 and 229; (40) SEQ ID NOS: 168 and 251; (41) SEQ ID NOS: 171 and 248 (42) SEQ ID NOS: 138 and 220; (43) SEQ ID NOS: 160 and 241; (44) SEQ ID NOS: 164 and 252; (45) SEQ ID NOS: 139 and 231; (46) SEQ ID NOS: 150 and 223; (47) SEQ ID NOS: 144 and 233; (48) SEQ ID NOS: 170 and 252; (49) SEQ ID NOS: 163 and 242; (50) SEQ ID NOS: 150 and 228; (51) SEQ ID NOS: 147 and 226; (52) SEQ ID NOS: 140 and 217; (53) SEQ ID NOS: 172 and 247; (54) SEQ ID NOS: 143 and 230; (55) SEQ ID NOS: 139 and 224; (56) SEQ ID NOS: 150 and 235; (57) SEQ ID NOS: 184 and 253; (58) SEQ ID NOS: 168 and 255; (59) SEQ ID NOS: 175 and 255; (60) SEQ ID NOS: 173 and 253, (61) SEQ ID NOS: 164 and 255 (62) SEQ ID NOS: 177 and 253; (63) SEQ ID NOS: 169 and 253; (64) SEQ ID NOS: 183 and 255; (65) SEQ ID NOS: 172 and 245; (66) SEQ ID NOS: 166 and 254; (67) SEQ ID NOS: 178 and 254, (68) SEQ ID NOS: 165 and 255; (69) SEQ ID NOS: 174 and 253; (70) SEQ ID NOS:171 and 254; (71) SEQ ID NOS: 160 and 255; (72) SEQ ID NOS: 172 and 246; (73) SEQ ID NOS: 175 and 254; (74) SEQ ID NOS: 161 and 253; (75) SEQ ID NOS: 159 and 255; (76) SEQ ID NOS: 167 and 253, (77) SEQ ID NOS: 183 and 254, (78) SEQ ID NOS: 181 and 255; (79) SEQ ID NOS: 150 and 240; (80) SEQ ID NOS: 179 and 253; (81) SEQ ID NOS: 172 and 254; (82) SEQ ID NOS: 176 and 253; (83) SEQ ID NOS: 183 and 253; (84) SEQ ID NOS: 166 and 255; (85) SEQ ID NOS: 154 and 239; (86) SEQ ID NOS: 178 and 253; (87) SEQ ID NOS:153 and 239; (88) SEQ ID NOS: 152 and 239; (89) SEQ ID NOS: 185 and 254; (90) SEQ ID NOS: 171 and 255; (91) SEQ ID NOS: 155 and 239; (92) SEQ ID NOS: 182 and 255; (93) SEQ ID NOS:180 and 254; (94) SEQ ID NOS: 172 and 244; and (95) SEQ ID NOS: 183 and 253. In a particular embodiment. the forward and reverse primer pair are sleeted from the group consisting of: (1) SEQ ID NOS:256 and 292; (2) SEQ ID NOS: 257 and 292; (3) SEQ ID NOS: 257 and 293; (4) SEQ ID NOS: 258 and 292; (5) SEQ ID NOS: 259 and 292; (6) SEQ ID NOS: 260 and 292; (7) SEQ ID NOS: 261 and 292, (8) SEQ ID NOS: 262 and 292; (9) SEQ ID NOS: 263 and 293, (10) SEQ ID NOS: 264 and 294; (11) SEQ ID NOS: 264 and 295; (12) SEQ ID NOS: 264 and 296; (13) SEQ ID NOS: 264 and 297; (14) SEQ ID NOS: 264 and 298; (15) SEQ ID NOS: 264 and 299) (16) SEQ ID NOS: 264 and 300; (17) SEQ ID NOS: 265 and 294; (18) SEQ ID NOS: 265 and 301; (19) SEQ ID NOS: 265 and 302; (20) SEQ ID NOS: 265 and 303; (21) SEQ ID NOS: 265 and 304; (22) SEQ ID NOS: 265 and 305; (23) SEQ ID NOS: 265 and 299; (24) SEQ ID NOS: 265 and 306; (25) SEQ ID NOS: 265 and 307; (26) SEQ ID NOS: 266 and 294; (27) SEQ ID NOS: 266 and 301; (28) SEQ ID NOS 266 and 296; (29) SEQ ID NOS: 266 and 303; (30) SEQ ID NOS: 266 and 298; (31) SEQ ID NOS: 266 and 308; (32) SEQ ID NOS: 266 and 309; (33) SEQ ID NOS: 266 and 300; (34) SEQ ID NOS: 267 and 294; (35) SEQ ID NOS: 267 and 301; (36) SEQ ID NOS: 267 and 296; (37) SEQ ID NOS: 267 and 303; (38) SEQ ID NOS: 267 and 298; (39) SEQ ID NOS: 267 and 305; (40) SEQ ID NOS: 267 and 299; (41) SEQ ID NOS: 267 and 310; (42) SEQ ID NOS: 267 and 300; (43) SEQ ID NOS: 268 and 3089 (44) SEQ ID NOS: 268 and 299; (45) SEQ ID NOS: 268 and 310; (46) SEQ ID NOS: 268 and 300; (47) SEQ ID NOS: 269 and 311 (48) SEQ ID NOS: 269 and 312; (49) SEQ ID NOS: 269 and 313; (50) SEQ ID NOS: 269 and 314; (51) SEQ ID NOS: 269 and 315; (52) SEQ ID NOS: 269 and 316; (53) SEQ ID NOS: 269 and 306; (54) SEQ ID NOS: 270 and 294; (55) SEQ ID NOS: 270 and 317; (56) SEQ ID NOS: 270 and 303; (57) SEQ ID NOS: 270 and 298; (58) SEQ ID NOS: 270 and 308; (59) SEQ ID NOS: 270 and 309; (60) SEQ ID NOS: 270 and 310; (61) SEQ ID NOS: 270 and 300; (62) SEQ ID NOS:271 and 294; (63) SEQ ID NOS: 271 and 301; (64) SEQ ID NOS: 271 and 296; (65) SEQ ID NOS: 271 and 303; (66) SEQ ID NOS: 271 and 298; (67) SEQ ID NOS: 271 and 305, (68) SEQ ID NOS: 271 and 318; (69) SEQ ID NOS: 271 and 309; (70) SEQ ID NOS: 271 and 319; (71) SEQ ID NOS: 271 and 300; (72) SEQ ID NOS: 272 and 320; (73) SEQ ID NOS: 272 and 310; (74) SEQ ID NOS: 272 and 307; (75) SEQ ID NOS: 272 and 321; (76) SEQ ID NOS: 272 and 299; (77) SEQ ID NOS: 273 and 322; (78) SEQ ID NOS: 273 and 323; (79) SEQ ID NOS: 273 and 324; (80) SEQ ID NOS: 274 and 325; (81) SEQ ID NOS: 274 and 326; (82) SEQ ID NOS: 274 and 327; (83) SEQ ID NOS: 274 and 328; (84) SEQ ID NOS: 275 and 325; (85) SEQ ID NOS: 275 and 323; (86) SEQ ID NOS: 275 and 327; (87) SEQ ID NOS: 276 and 323; (88) SEQ ID NOS: 276 and 326; (89) SEQ ID NOS: 277 and 325; (90) SEQ ID NOS: 277 and 326; (91) SEQ ID NOS: 277 and 327; (92) SEQ ID NOS: 278 and 325; (93) SEQ ID NOS: 278 and 323; (94) SEQ ID NOS: 278 and 326; (95) SEQ ID NOS: 278 and 327; (96) SEQ ID NOS: 279 and 325; (97) SEQ ID NOS: 279 and 326; (98) SEQ ID NOS: 279 and 327; (99) SEQ ID NOS: 279 and 328; (100) SEQ ID NOS: 280 and 325; (101) SEQ ID NOS: 280 and 326; (102) SEQ ID NOS: 280 and 327; (103) SEQ ID NOS: 280 and 328; (104) SEQ ID NOS: 281 and 325; (105) SEQ ID NOS: 281 and 323; (106) SEQ ID NOS: 281 and 327; (107) SEQ ID NOS: 281 and 328; (108) SEQ ID NOS: 329 and 331; (109) SEQ ID NOS: 330 and 321; and (110) SEQ ID NOS: 329 and 331. In a particular embodiment, step (3) is performed using one or more labeled probes comprising a sequence that hybridizes to the amplicon generated by the forward and reverse primer pair group of Tables 2, 3 or 4. In a particular embodiment, the sequence of the one or more labeled probes is selected from the group consisting of: SEQ ID NOS: 2, 4, 5, 11, 14, 16, 18, 24, 27, 38, 44, 41, 53, 56, 59, 61, 63, 64, 65, 69, 71, 77, 81, 83, 85, 86, 93, 96, 99, 101, 102, 108, 110, 122, 124, 127, 133-134, 186-214, 282-291, and 332-333. In a particular embodiment, the one or more labeled probes is fluorescently labeled and the step of detecting the binding of the one or more labeled probes to the amplified PCR product comprises measuring the fluorescence of the sample. In a particular embodiment, the one or more labeled probes is fluorescently labeled and the step of quantitating the binding of the one or more labeled probes to the amplified PCR product comprises measuring the fluorescence of the sample. In a particular embodiment, the sample is selected from the group consisting of: blood, serum, plasma, sputum, urine, stool, skin, cerebrospinal fluid, saliva, semen, seminal fluid, gastric secretions, tears, oropharyngeal swabs, nasopharyngeal swabs, throat swabs, nasal aspirates, nasal wash, renal tissue and fluid therefrom including perfusion media, fluids collected from the ear, eye, mouth, and respiratory airways, neoplastic tissue obtained from biopsies, tissues utilized for transplantation between individuals and/(or animal derived tissues utilized for transplantation into a human recipient, fluids and cells derived from the culturing of human cells, human stem cells, human cartilage and fibroblasts. In a particular embodiment, the sample is a human or a mammalian sample to be utilized in or on a human subject.

One embodiment is directed to a polynucleotide comprising a nucleotide sequence selected from the group consisting of: SEQ ID NOS: 1-334, for isolating nucleic acid sequences derived from any BK virus genome.

One embodiment is directed to a polynucleotide comprising a nucleotide sequence selected from the group consisting of: SEQ ID NOS: 1-334, for sequencing nucleic acid sequences derived from any BK virus genome.

One embodiment is directed to a polynucleotide probe comprising SEQ ID NO:135 for binding to an internal control target sequence comprising SEQ ID NO:335.

One embodiment is directed to a polynucleotide probe comprising SEQ ID NO:334 for binding to an internal control target sequence comprising SEQ ID NO:336. In a particular embodiment, the probe monitors the efficiency of extraction of nucleic acids from a sample, and the amplification step of PCR.

One embodiment is directed to a method for detecting a BKV-associated condition or disease, comprising: (1) contacting at least one forward and reverse primer pair listed in Tables 2, 3 or 4 to a sample; (2) conducting a polymerase chain reaction; and (3) detecting and/or quantitating the generation of an amplified PCR product, wherein the generation of an amplified PCR product indicates the presence of BK virus in the sample. In a particular embodiment, the BKV-associated condition or disease is a BKV-associated neuropathy or BKV infection in a sample selected from the group consisting of: renal epithelial cells brain tissue neoplastic tissue obtained from biopsies, cerebrospinal fluid, and tissues utilized for transplantation.

### DETAILED DESCRIPTION

Described herein are nucleic acid primers and probes for detecting and quantitating viral genetic material, especially BK viruses, and methods and devices for designing and optimizing the respective primer and probe sequences. The present invention therefore provides a method for specifically detecting and quantitating the presence of any BK virus variant in a given sample using the primers and probes provided herein. Of particular interest in this regard is the ability of the disclosed primers and probes, as well as those that can be designed according to the disclosed methods, to specifically detect and quantitate all or a majority of currently identified strains and variants of the BK virus. The optimized primers and probes of the invention are useful, therefore, for identifying, diagnosing and quantitating the causative or contributing agent of disease caused by BK virus whereupon an appropriate treatment can then be administered to the individual and steps taken to eradicate the virus, “BK”, “BK virus” and “BKV” are exchangeable terms used herein to refer to the BK virus.

The present invention provides one or more pairs of PCR primers that can anneal to all known BKV variants and thereby amplify a PCR product from a biological sample. The present invention provides a first PCR primer and a second PCR primers each of which comprises a nucleotide sequence designed according to the inventive principles disclosed herein, which are used together to positively identify the presence of the BK virus in a sample regardless of the actual nucleotide composition of the infecting BK variant(s). The generation of an amplified PCR product or products from a sample using the primer pairs disclosed herein is diagnostically indicative of a variant of the BK virus being present in the sampled material and at least diagnostically indicative of the presence of BK virus infection. Of note, each of the primer sequences can be used as a probe to detect viral variants.

Also provided by the present invention are probes that hybridize to amplified PCR products or unamplified sample sequences. A probe can be labeled, for example, such that when it binds to an internal PCR product target sequence, or after it has been cleaved after binding, a fluorescent signal is emitted that is detectable under various spectroscopy and light-measurement apparatuses. The use of a labeled probe, therefore, can enhance the sensitivity of the PCR-based amplification of variant BKV DNA because it permits the detection of virus DNA at low template concentrations that might not be conducive to visual detection as a gel-stained PCR product.

Primers and probes of the invention are sequences that anneal to a viral genomic sequence, e.g., BKV (the “target”), The target sequence can be, for example, a viral genome or a subset, “region”, of a viral genome, In one embodiment, the entire genomic sequence can be “scanned” for optimized primers and probes useful for detecting viral variants. In other embodiments, particular regions of the viral genome can be scanned, e.g., regions that are documented in the literature as being useful for detecting multiple variants, regions that are conserved, or regions where sufficient information is available in, for example, a public database, with respect to viral variants.

Sets of primers and probes are generated based on the target to be detected. The set of all possible primers and probes can include, for example, sequences that include the variability at every site based on the known viral variants, or the primers and probes can be generated based on a consensus sequence of the target. The primers and probes are generated such that the primers and probes are able to anneal to a particular variant or a consensus sequence under high stringency conditions. For example, one of skill in the art recognizes that for any particular sequence, it is possible to provide more than one oligonucleotide sequence that will anneal to the particular target sequence, even under high stringency conditions. The set of primers and probes to be sampled for the purposes of the present invention includes, for example, all such oligonucleotides for all viral variant sequence. Alternatively, the primers and probes includes all such oligonucleotides for a given consensus sequence for a target.

Typically, stringent hybridization and washing conditions are used for nucleic acid molecules over about 500 bp. Stringent hybridization conditions include a solution comprising about 1 M Na⁺ at 25° C. to 30° C. below the Tm; e.g., 5×SSPE, 0.5% SDS, at 65° C. see, Ausubel, et al., Current Protocols in Molecular Biology, Greene Publishing, 1995; Sambrook et al., Molecular Cloning A Laboratory Manual, Cold Spring Harbor Press, 1989). Tm is dependent on both the G+C content and the concentration of salt ions, e.g., Na⁺ and K⁺. A formula to calculate the Tm of nucleic acid molecules greater than about 500 bp is Tm=81.5+0.41(%(G+C))−log₁₀[Na⁺]. Washing conditions are generally performed at least at equivalent stringency conditions as the hybridization. If the background levels are high, washing can be performed at higher stringency such as around 15° C. below the Tm.

The set of primers and probes, once determined as described above, are optimized for hybridizing to a plurality of viral variants by employing scoring and/or ranking steps that provide a positive or negative preference or “weight” to certain nucleotides in a target nucleic acid variant sequence. For example, if a consensus sequence is used to generate the full set of primers and probes, then a particular primer sequence is scored for its ability to anneal to the corresponding sequence of every known native variant sequence. Even if a probe were originally generated based on a consensus, therefore, the validation of the probe is in its ability to specifically anneal and detect every or a large majority of variant viral sequences. The particular scoring or ranking steps performed depend upon the intended use for the primer and/or probe, the particular target nucleic acid sequence, and the number of variants of that target nucleic acid sequence. The methods of the invention provide optimal primer and probe sequences because they hybridize to all or a subset of BK virus variants. Once optimized oligonucleotides are identified that can anneal to viral variants, the sequences can then further be optimized for use, for example, in conjunction with another optimized sequence as a “primer pair” or for use as a probe.

Primer or probe sequences can be ranked according to specific hybridization parameters or metrics that assign a score value indicating their ability to anneal to viral variants under highly stringent conditions. Where a primer pair is being scored, a “first” or “forward” primer is scored and the “second” or “reverse”-oriented primer sequences can be optimized similarly but with potentially additional parameters, followed by an optional evaluation for primer dimmers for example, between the forward and reverse primers.

The scoring or ranking steps that are used in the methods of determining the primers and probes described herein include, for example, the following parameters: a target sequence score for the target nucleic acid sequencers), e.g., the PriMD® score; a mean conservation score for the target nucleic acid sequence(s); a mean coverage score for the target nucleic acid sequence(s); 100% conservation score of a portion (e.g., 5′ end, center, 3′ end) of the target nucleic acid sequence(s); a species score; a strain score; a subtype score; a serotype score; an associated disease score; a year score; a country of origin score; a duplicate score; a patent score; and a minimum qualifying score. Other parameters that are used include, for example, the number of mismatches, the number of critical mismatches (e.g., mismatches that result in the predicted failure of the sequence to anneal to a target sequence), the number of native variant sequences that contain critical mismatches, and predicted Tm values. The term “Tm” refers to the temperature at which a population of double-stranded nucleic acid molecules becomes half-dissociated into single strands. Methods for calculating the Tm of nucleic acids are well known in the art (Berger and Kimmel (1987) Meth. Enzymol., Vol. 152: Guide To Molecular Cloning Techniques, San Diego: Academic Press, Inc. and Sambrook et al, (1989) Molecular Cloning. A Laboratory Manual, (2nd ed. ) Vols. 1-3, Cold Spring Harbor Laboratory).

The resultant scores represent steps in determining nucleotide or whole target nucleic acid sequence preference, while tailoring the primer and/or probe sequences so that they hybridize to a plurality of target nucleic acid variants. The methods of the invention also can comprise the step of allowing for one or more nucleotide changes when determining identity between the candidate primer and probe sequences and the target nucleic acid variant sequences, or their complements.

In another embodiment, methods for determining the primers and probes comprise the steps of comparing the candidate primer and probe nucleic acid sequences to “exclusion nucleic acid sequences” and then rejecting those candidate nucleic acid sequences that share identity with the exclusion nucleic acid sequences. In another embodiment, the methods of the invention comprise the steps of comparing the candidate primer and probe nucleic acid sequences to “inclusion nucleic acid sequences” and then rejecting those candidate nucleic acid sequences that do not share identity with the inclusion nucleic acid sequences.

In an embodiment, optimizing primers and probes comprises using a polymerase chain reaction (PCR) penalty score formula comprising at least one of a weighted sum of: primer Tm−optimal Tm; difference between primer Tms; amplicon length−minimum amplicon length; and distance between the primer and a TaqMan® probe. The optimizing step also can comprise determining the ability of the candidate sequence to hybridize with the most target nucleic acid variant sequences (e.g., the most target organisms or genes). In another embodiment, the selecting or optimizing step comprises determining which sequences have mean conservation scores closest to 1, wherein a standard of deviation on the mean conservation scores is also compared.

In other embodiments, methods for determining the primers and probes comprise the step of evaluating which target nucleic acid variant sequences are hybridized by an optimal forward primer and an optimal reverse primer, for example, by determining the number of base differences between target nucleic acid variant sequences in a database. For example, the evaluating step can comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; (4) and/or determining the secondary structure of the forward primer, reverse primer, and/or target. In an embodiment, the evaluating step includes evaluating whether the forward primer sequence, reverse primer sequences and/or probe sequence hybridizes to sequences in the database other than the nucleic acid sequences that are representative of the target variants.

Described herein are polynucleotides that have preferred primer and probe qualities. These qualities are specific to the sequences of the optimized probes, however, one of skill in the art would recognize that other molecules with similar sequences could also be used. The oligonucleotides provided herein comprise a sequence that shares at least about 60-70% identity with a sequence described in Tables 2, 3 or 4. In addition, the sequences can be incorporated into longer sequences, provided they function to specifically anneal to and identify viral variants. In another embodiment, a nucleic acid sequence share s at least about 71 %, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91 %, about 92%, about 93%, about 94%, about 95%, about 96%, about 97%, about 98%, about 99%, or about 100% identity with the sequences of Tables 2, 3 or 4 or complement thereof The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art. The primer and/or probe nucleic acid sequences of the invention are complementary to the target nucleic acid sequence. The probe and/or primer nucleic acid sequences of the invention are optimal for identifying numerous variants of a target nucleic acid, e.g., from BKV pathogen. In an embodiment, the nucleic acids of the invention are primers for the synthesis (e.g., amplification) of target nucleic acid variants and/or probes for identification, isolation, detection, or analysis of target nucleic acid variants, e.g., an amplified target nucleic acid variant that is amplified using the primers described herein.

The present polynucleotides hybridize with more than one viral variant (variants as determined by differences in their genomic sequence). The probes and primers provided herein can, for example, allow for the detection and quantitation of currently identified (an yet to be identified) viral variants or a subset thereof. The primers and probes of the present invention, depending on the variant sequencers), allow for the detection and quantitation of previously unidentified viral variants. The methods of the invention provide for optimal primers and probes, and sets thereof, and combinations of sets thereof, which can hybridize with a larger number of target variants than available primers and probes.

In other aspects, the invention also provides vectors (e.g., plasmid, phage, expression), cell lines (e.g., mammalian, insect, yeast, bacterial), and kits comprising any of the sequences of the invention described herein. The invention further provides known or previously unknown target nucleic acid variant sequences that are identified, for example, using the methods of the invention. In an embodiment, the target nucleic acid variant sequence is an amplification product. In another embodiment, the target nucleic acid variant sequence is a native or synthetic nucleic acid. The primers, probes, and target nucleic acid variant sequences vectors, cell lines, and kits can have any number of uses, such as diagnostic investigative, confirmatory, monitoring, predictive or prognostic.

A diagnostic kit is provided by the present invention that comprises one or more of the polynucleotides described herein, which are useful for detecting and quantitating BKV infection in an individual and/or from a sample. An individual can be a human male, human female, human adult, human child, or human fetus. An individual can also be any mammal, reptile, avian, fish or amphibian. Hence an individual can be a mouse, rat, sheep, dog, simian, horse, cattle, chicken, porcine, lamb, bird or fish. A sample includes any item, surface, material, clothing or environment, for example, sewage or water treatment plants, in which it is desirable to test for the presence of BKV variants. The present invention includes, for example, the testing of door handles, faucets, table surfaces, elevator buttons, chairs, toilet seats, sinks, kitchen surfaces, children's cribs, bed linen, pillows, keyboards and so on, for the presence of BKV variants.

A probe of the present invention can comprise a label such as, for example, a fluorescent label, a chemiluminescent label, a radioactive label, biotin, gold, dendrimers, aptamer, enzymes, proteins, quenchers and molecular motors. In an embodiment, the probe is a hydrolysis probe, such as, for example, a TaqMan® probe. In other embodiments, the probes of the invention are molecular beacons, SYBR Green primers, or fluorescence energy transfer (FRET) probes.

Polynucleotides of the present invention include not only primers that are useful for conducting the aforementioned PCR amplification reactions, but also include polynucleotides that are attached to a solid support, such as, for example, a microarray, multiwell plate, column, bead, glass slide, polymeric membranes glass microfiber, plastic tubes, cellulose, and carbon nanostructures. Hence, detection of BK virus variants can be performed by exposing such a polynucleotide-covered surface to a sample such that the binding of a complementary variant DNA sequence to a surface-attached polynucleotide elicits a detectable signal or reaction.

Polynucleotides also include primers for isolating and sequencing nucleic acid sequences derived from any identified or yet to be isolated and identified BK virus genome.

One embodiment of the invention uses solid support-based oligonucleotide hybridization methods to detect gene expression. Solid support-based methods suitable for practicing the present invention are widely known and are described (PCT application WO 95/11755; Huber et al., Anal. Biochem., 299:24, 2001; Meiyanto et al, Biotechniques, 31:406, 2001; Relogio et al., Nucleic Acids Res., 30:e51 2002; the contents of which are incorporated herein by reference in their entirety). Any solid surface to which oligonucleotides can be bound, covalently or non-covalently, can be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one common approach, the solid support is coated with a polymeric layer that provides linker arms with a plurality of reactive ends/sites. A common example of this type is glass slides coated with polylysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption ionization time of flight (MALDI-TOF) mass spectrometry. Therefore, streptavidin can be covalently attached to a solid support, and a biotinylated nucleic acid molecule will bind to the streptavidin-coated surface. In one version of this method, an amino-coated silicon wafer is reacted with the n-hydroxysuccinimido-ester of biotin and complexed with streptavidin, Biotinylated oligonucleotides are bound to the surface at a concentration of about 20 fmol DNA per mm².

Alternatively, one can directly bind DNA to the support using carbodiimides, for example. In one such method, the support is coated with hydrazide groups, then treated with carbodiimide. Carboxy-modified nucleic acid molecules are then coupled to the treated support Epoxide-based chemistries are also being employed with amine modified oligonucleotides. Other chemistries for coupling nucleic acid molecules to solid substrates are known to those of skill in the art.

The nucleic acid molecules, e.g., the primers and probes of the present invention, must be delivered to the substrate material which is suspected of containing or is being tested for the presence and number of BK virus molecules. Because of the miniaturization of the arrays delivery techniques must be capable of positioning very small amounts of liquids in very small regions very close to one another and amenable to automation. Several techniques and devices are available to achieve such delivery. Among these are mechanical mechanisms (e.g., arrayers from GeneticMicroSystems, MA, USA) and inkjet technology. Very fine pipets can also be used.

Other formats are also suitable within the context of this invention. For example a 96-well format with fixation of the nucleic acids to a nitrocellulose or nylon membrane may also be employed.

After the nucleic acid molecules have been bound to the solid support, it is often useful to block reactive sites on the solid support that are not consumed in binding to the nucleic acid molecule. In the absence of the blocking step, excess primers and/or probes can, to some extent, bind directly to the solid support itself, giving rise to non-specific binding. Non-specific binding can sometimes hinder the ability to detect low levels of specific binding. A variety of effective blocking agents (e.g., milk powder serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays, that can be used to simultaneously observe the expression of a number of BKV and BKV variant genes. Oligonucleotide arrays comprise two or more oligonucleotide probes provided on a solid support, wherein each probe occupies a unique location on the support. The location of each probe can be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting gene expression can be made and used according to conventional techniques described (Lockhart et al., Nat. Biotech., 14:1 675-1680, 1996; McGall et al., Proc. Natl. Acad. Sci. USA, 93:13555, 1996; Hughes et al, Nat. Biotechnol., 19:342, 2001). A variety of oligonucleotide array designs are suitable for the practice of this invention.

Generally, a detectable molecule, also referred to herein as a label, can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR, PCR primers used in generating the probes are chosen, for example, based on the sequences of Tables 2, 3 or 4. In one embodiment, oligonucleotide control probes also are used. Exemplary control probes can fall into at least one of three categories referred to herein as (1) normalization controls, (2) expression level controls and (3) negative controls. In microarray methods, one or more of these control probes can be provided on the array with the inventive cell cycle gene-related oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. The normalization controls also allow for the semi-quantification of the signals from other features on the microarray. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (e.g., without forming secondary structures) and do not match any test probes. In one embodiment, the normalization controls are mammalian genes.

“Negative control” probes are not complementary to any of the test oligonucleotides (e.g., the inventive cell cycle gene-related oligonucleotides), normalization controls, or expression controls. In one embodiment, the negative control is a mammalian gene which is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In a one embodiment, background is calculated as the average hybridization signal intensity for the lowest 5 to 10 percent of the oligonucleotide probes being used, or, where a different background signal is calculated for each target gene, for the lowest 5 to 10 percent of the probes for each gene. Where the oligonucleotide probes corresponding to a particular BK virus target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. The invention also provides methods of labeling nucleic acid molecules with cleavable mass spectrometry tags (CMST; U.S. Pat. No. 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect gene expression. These methods include reverse-traniscriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol., 3:RESEARCH0034, 2002; Stein, Cell Mol. Life Sci. 59:1235, 2002.

The forward and reverse amplification primers and internal hybridization probe is designed to hybridize specifically and uniquely with one nucleotide sequence derived from the transcript of a target gene. In one embodiment, the selection criteria for primer and probe sequences incorporates constraints regarding nucleotide content and size to accommodate TaqMan® requirements. SYBR Green® can be used as a probe-less Q-RTPCR alternative to the TaqMan®-type assay, discussed above (ABI Prism® 7900 Sequence Detection System User Guide Applied Biosystems, chap. 1-8, App. A-F. (2002)). A device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867).

The primers and probes of the present invention may anneal to or hybridize to various BKV genetic material or genetic material derived therefrom, such as RNA, DNA, cDNA, or a PCR product.

A “sample” that is tested for the presence of BK virus variant includes, but is not limited to, for example, blood, serum, plasma, sputum, urine, stool, skin cerebrospinal fluid, saliva, gastric secretions, hair, renal tissue and fluid therefrom, kidney or other tissues and fluid therefrom, brain tissue and fluid therefrom, neoplastic tissue obtained from biopsies and tear fluid. A sample can be obtained by an oropharyngeal swab, nasopharyngeal swab, throat swab, nasal aspirate, nasal wash, or fluid collected from the ear, eye, mouth, or respiratory airway. The tissue sample may be fresh, fixed, preserved, or frozen. A sample also includes any item surface, material or clothing, or environment for example, sewage or water treatment plants, in which it may be desirable to test for the presence of BKV variants. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of BKV variants.

The target nucleic acid variant that is amplified may be RNA or DNA or a modification thereof. Thus, the amplifying step can comprise isothermal or non-isothermal reaction such as polymerase chain reaction Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isothermal amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen a molecular weight tag, and a modifier of probe Tm. The detection step can also comprise the incorporation of a label (e.g. fluorescent or radioactive) during an extension reaction. The detecting step comprises measuring fluorescence, mass, charge and/or chemiluminescence.

Hybridization can be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Scoring a Set of Predicted Annealing Oligonucleotides

Each of the sets of primers and probes selected is ranked by a combination of methods as individual primers and probes and as a primer/probe set. This involves one or more methods of ranking (e.g., joint ranking, hierarchical ranking, and serial ranking) where sets of primers and probes are eliminated or included based on any combination of the following criteria, and a weighted ranking again based on any combination of the following criteria, for example: (A) Percentage Identity to Target Variants; (B) Conservation Score; (C) Coverage Score; (D) Strain/Subtype/Serotype Score; (E) Associated Disease Score; (F) Duplicates Sequences Score; (G) Year and Country of Origin Score; (H) Patent Score, and (I) Epidemiology Score.

### (A) Percentage Identity

A percentage identity score is based upon the number of target nucleic acid variant (e.g., native) sequences that can hybridize with perfect conservation (the sequences are perfectly complimentary) to each primer or probe of a primer pair and probe set. If the score is less than 100%, the program ranks additional primer pair and probe sets that are not perfectly conserved. This is a hierarchical scale for percent identity starting with perfect complimentarity, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus sequence and these scores represent how many of the target nucleic acid variants sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid variant sequences have the same base (either an A, C, G, or T/U) at that position. If there is an equal number of bases (e.g., 50% A & 50% T) at a position, it is identified with an N.

(ii) Candidate Primer/Probe Sequence Conservation

An overall conservation score is generated for each candidate primer or probe sequence that represents how many of the target nucleic acid variant sequences will hybridize to the primers or probes. A candidate sequence that is perfectly complimentary to all the target nucleic acid variant sequences will have a score of 1.0 and rank the highest. For example, illustrated below in Table 1 are three different 10-base candidate probe sequences that are targeted to different regions of a consensus target nucleic acid variant sequence. Each candidate probe sequence is compared to a total of 10 native sequences.

TABLE 1

```
#1. A A A C A C G T G C
        0.7 1.0 1.0 1.0 1.0 1.0 1.0 1.0 1.0 1.0
SEQ ID NO:337
→Number of target nucleic acid variant sequences
that are perfectly complimentary-7. Three out of
the ten sequences do not have an A at position 1.
#2. C C T T G T T C C A
        1.0 0.9 1.0 0.9 0.9 1.0 1.0 1.0 1.0 1.0
SEQ ID NO:338
→Number of target nucleic acid variant sequences
that are perfectly complimentary-7, 8, or 9. At
least one tarqet nucleic acid variant does not
have a C at position 2, T at position 4, or G at
position 5. These differences may all be on one
target nucleic acid variant molecule or may be on
two or three separate molecules.
#3. C A G G G A C G A T
        1.0 1.0 1.0 1.0 1.0 0.9 0.8 1.0 1.0 1.0
SEQ ID NO:339
→Number of target nucleic acid variant sequences
that are perfectly complimentary-7 or 8. At least
one target nucleic acid variant does not have an A
at position 6 and at least two target nucleic acid
variant do not have a C at position 7. These
differences may all be on one target nucleic acid
variant molecule or may be on two separate
molecules.
```

A simple arithmetic mean for each candidate sequence would generate the same value of 0.97. The number of target nucleic acid variant sequences identified by each candidate probe sequence however, can be very different. Sequence #1 can only identify 7 native sequences because of the 0.7 (out of 1.0) score by the first base-A. Sequence #2 has three bases each with a score of 0.9, each of these could represent a different or shared target nucleic acid variant sequence. Consequently, Sequence #2 can identify 7, 8 or 9 target nucleic acid variant sequences. Similarly, Sequence #3 can identify 7 or 8 of the target nucleic acid variant sequences. Sequence #2 would, therefore, be the best choice if all the three bases with a score of 0.9 represented the same 9 target nucleic acid variant sequences.

(iii) Overall Conservation Score of the Primer and Probe Set—Percent Identity

The same method described in (ii) when applied to the complete primer pair and probe set will generate the percent identity for the set (see A above). For example, using the same sequences illustrated above, if Sequences #1 and #2 are primers and Sequence #3 is a probe, then the percent identity for the target can be calculated from how many of the target nucleic acid variant sequences are identified with perfect complimentarity by all three primer/probe sequences. The percent identity could be no better than 0.7 (7 out of 10 target nucleic acid variant sequences) but as little as 0.1 if each of the degenerate bases reflects a different target nucleic acid variant sequence. Again, an arithmetic mean of these three sequences would be 0.97. As none of the above examples were able to capture all the target nucleic acid variant sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complimentary to all the known native sequences that were used to generate the consensus sequence. If there is no such candidate, then the sets are ranked according to how many degenerate bases can be accepted and still hybridize to just the target sequence during the PCR and yet identify all the native sequences.

The hybridization conditions, for TaqMan® as an example are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers and probe set. The rules can be as simple as counting the number of degenerate bases to more sophisticated approaches based on exploiting the PCR criteria used by the PriMD® software. Each target nucleic acid variant sequence has a value or weight (see Score assignment above). If the failed target nucleic acid variant sequence is medically valuable, the primer/probe set is rejected. This in silico analysis provides a degree of confidence for a given genotype and is important when new sequences are added to the databases. New target nucleic acid variant sequences are automatically entered into both the “include” and “exclude” categories. Published primer and probes will also be ranked by the PriMD software.

(iv) Position (5′ to 3′) of the Base Conservation Score

In an embodiment, primers do not have bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria is different. For example, with a TaqMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TaqMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TaqMan® probe. If there are two or more degenerate bases in a primer and probe set the ranking will initially select the sets where the degeneracies occur on different sequences.

### B. Coverage Score

The total number of aligned sequences is considered under coverage score. A value is assigned to each position based on how many times that position has been reported or sequenced. Alternatively, coverage can be defined as how representative the sequences are of the known strains, subtypes etc., or their relevance to a certain diseases. For example, the target nucleic acid variant sequences for a particular gene may be very well conserved and show complete coverage but certain strains are not represented in those sequences.

A sequence is included if it aligns with any part of the consensus sequence, which is usually a whole gene or a functional unit, or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

### C. Strain/Subtype/Serotype Score

A value is assigned to each strain or subtype or serotype based upon its relevance to a disease. For example, strains of BKV that are linked to high frequencies of infection will have a higher score than strains that are generally regarded as benign. The score is based upon sufficient evidence to automatically associate a particular strain with a disease. For example, certain strains of adenovirus are not associated with diseases of the upper respiratory system. Accordingly, there will be sequences included in the consensus sequence that are not associated with diseases of the upper respiratory system.

### D. Associated Disease Score

The associated disease score pertains to strains that are not known to be associated with a particular disease (to differentiate from D above). Here, a value is assigned only if the submitted sequence is directly linked to the disease and that disease is pertinent to the assay.

### E. Duplicate Sequences Score

If a particular sequence has been sequenced more than once it will have an effect on representation, for example, a strain that is represented by 12 entries in GenBank of which six are identical and the other six are unique. Unless the identical sequences can be assigned to different strains/subtypes (usually by sequencing other gene or by immunology methods) they will be excluded from the scoring.

### F. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. Certain divergent strains from more obscure countries or sources may also be less relevant to the locations that will likely perform clinical tests, or may be more important for certain countries (e.g., North America, Europe, or Asia).

### G. Patent Score

Candidate target variant sequences published in patents are searched electronically and annotated such that patented regions are excluded. Alternatively, candidate sequences are checked against a patented sequence database.

### H. Minimum Qualifying Score

The minimum qualifying score is determined by expanding the number of allowed mismatches in each set of candidate primers and probes until all possible native sequences are represented (e.g., has a qualifying hit).

### I. Other

A score is given to based on other parameters, such as relevance to certain patients (e.g., pediatrics, immunocompromised) or certain therapies (e.g. target those strains that respond to treatment) or epidemiology. The prevalence of an organism/strain and the number of times it has been tested for in the community can add value to the selection of the candidate sequences. If a particular strain is more commonly tested then selection of it would be more likely. Strain identification can be used to select better vaccines.

### Example 2

### Primer/Probe Evaluation

Once the candidate primers and probes have received their scores and have been ranked, they are evaluated using any of a number of methods such as, for example, BLAST analysis and secondary structure analysis.

### A. BLAST Analysis

The candidate primer/probe sets are submitted to BLAST analysis to check or possible overlap with any published sequences that might be missed by the Include/Exclude function. It also provides a useful summary.

### B. Secondary Structure

The methods of the present invention include analysis of nucleic acid secondary structure This includes the structures of the primers and/or probes as well as their intended target variant sequences. The methods and software of the invention predict the optimal temperatures for the annealing but assumes that the target (e.g. RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary stricture thereby preventing the annealing of the primer. Similarly, after denaturation of a double stranded DNA target (for example, an amplicon after PCR), the binding of the probe is dependent on there being no major secondary structure in amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds. NATO ASI Series, Kluwer Academic Publishers).

### C. Evaluating the Primer and Probe Sequences

The methods and software of the invention may also analyze any nucleic acid sequence to determine its suitability in a nucleic acid amplification-based assay. For example, it can accept a competitor's primer set and determine the following information: (1) How it compares to the primers of the invention (e.g., overall rank, PCR and conservation ranking, etc.); (2) How it aligns to the exclude libraries (e.g., assessing cross-hybridization)—also used to compare primer and probe sets to newly published sequences; and (3) If the sequence has been previously published This step requires keeping a database of sequences published in scientific journals, posters, and other presentations.

### Example 3

### Multiplexing

The Exclude/Include capability is ideally suited for designing multiplex reactions. The parameters for designing multiple primer and probe sets adhere to a more stringent set of parameters than those used for the initial Exclude/Include function. Each set of primers and probe, together with the resulting a amplicon is screened against the other sets that constitute the multiplex reaction. As new targets are accepted their sequences are automatically added to the Exclude category.

The database is designed to interrogate the online databases to determine and acquire, if necessary, any new sequences relevant to the targets. These sequences are evaluated against the optimal primer/probe set. If they represented a new genotype or strain then a multiple sequence alignment may be required.

### Example 4

### Sequences Identified for Detecting BKV

The set of primers and probes were then scored according to the methods described herein to identify the optimized primers and probes of Tables 2 3 and 4. It should be noted that the primers, as they are sequences that anneal to a plurality of all identified or unidentified BKV variants, can also be used as probes either in the presence or absence of amplification of a sample.

TABLE 2
Optimized Primers and Probes for Detecting BKV

| | | | |
|---|---|---|---|
| No. | Forward Primer | Probe Group | Reverse Primer |

| | | | |
|---|---|---|---|
| 1 | ACAGCCCAGTCCCAGGTAAT | ATACATAGGCTGCCCATCCACTCTC | AGTCTTTCTGTGCCATCAAACA |
| | (SEQ ID NO: 1) | TGG | (SEQ ID NO: 3) |
| | | (SEQ ID NO: 2) | |
| 2 | ACAGCCCAGTCCCAGGTAAT | ATACCATACATAGGCTGCCCATCCA | AGTCTTTCTGTGCCATCAAACA |
| | (SEQ ID NO: 1) | CTCTCT | (SEQ ID NO: 3) |
| | | (SEQ ID NO: 4) | |
| 3 | ACAGCCCAGTCCCAGGTAAT | CATACCATACATAGGCTGCCCATCC | AGTCTTTCTGTGCCATCAAACA |
| | (SEQ ID NO: 1) | ACTCT | (SEQ ID NO: 3) |
| | | (SEQ ID NO: 5) | |
| 4 | GGCCTATTTGGACAAAAACAAT | ATACATAGGCTGCCCATCCACTCTC | AGTCTTTCTGTGCCATCAAACA |
| | (SEQ ID NO: 6) | TGG | (SEQ ID NO: 3) |
| | | (SEQ ID NO: 2) | |
| 5 | GCCTATTTGGACAAAAACAATG | CATACCATACATAGGCTGCCCATCC | AGTCTTTCTGTGCCATCAAACA |
| | (SEQ ID NO: 7) | ACTCT | (SEQ ID NO: 3) |
| | | (SEQ ID NO: 5) | |
| 6 | CCTATTTGGACAAAAACAATGC | ATACCATACATAGGCTGCCCATCCA | AGTCTTTCTGTGCCATCAAACA |
| | (SEQ ID NO: 8) | CTCTCT | (SEQ ID NO: 3) |
| | | (SEQ ID NO: 4) | |
| 7 | AAGATTCGCCTGAGAAAAAGAT | ATACATAGGCTGCCCATCCACTCTC | AGTCTTTCTGTGCCATCAAACA |
| | (SEQ ID NO: 9) | TGG | (SEQ ID NO: 3) |
| | | (SEQ ID NO: 2) | |
| 8 | AAGATTCGCCTGAGAAAAAGAT | ATACCATACATAGGCTGCCCATCCA | AGTCTTTCTGTGCCATCAAACA |
| | (SEQ ID NO: 9) | CTCTCT | (SEQ ID NO: 3) |
| | | (SEQ ID NO: 4) | |
| 9 | AGGGGCAGCTCCCAAAAAG | CAAAGGAACCCGTGCAAGTGCCAAA | GATTTCCACAGGTTAGGTCCTCAT |
| | (SEQ ID NO: 10) | ACTACTAATAAA | (SEQ ID NO: 12) |
| | | (SEQ ID NO: 11) | |
| 10 | CAGGGGCAGCTCCCAAA | AAAGGAACCCGTGCAAGTGCCAAAA | GATTTCCACAGGTTAGGTCCTCAT |
| | (SEQ ID NO: 13) | CTACTAATAAA | (SEQ ID NO: 12) |
| | | (SEQ ID NO: 14) | |
| 11 | GGGCAGCTCCCAAAAAGC | AAAGGAACCCGTGCAAGTGCCAAAA | TAGATTTCCACAGGTTAGGTCCTCAT |
| | (SEQ ID NO: 15) | CTACTAAT | (SEQ ID NO: 17) |
| | | (SEQ ID NO: 16) | |
| 12 | GGGCAGCTCCCAAAAAGC | AAGGAACCCGTGCAAGTGCCAAAAC | GTAGATTTCCACAGGTTAGGTCCTCAT |
| | (SEQ ID NO: 15) | TACTAATAAA | (SEQ ID NO: 19) |
| | | (SEQ ID NO: 18) | |
| 13 | CAGGGGCAGCTCCCAAAA | AAGGAACCCGTGCAAGTGCCAAAAC | AGATTTCCACAGGTTAGGTCCTCAT |
| | (SEQ ID NO: 20) | TACTAATAAA | (SEQ ID NO: 21) |
| | | (SEQ ID NO: 14) | |
| 14 | GGGCAGCTCCCAAAAAGC | AAAGGAACCCGTGCAAGTGCCAAAA | AGTAGATTTCCACAGGTTAGGTCCTC |
| | (SEQ ID NO: 15) | CTACTAATAAA | AT |
| | | (SEQ ID NO: 14) | (SEQ ID NO: 22) |
| 15 | AGGGGCAGCTCCCAAAA | AAGGAACCCGTGCAAGTGCCAAAAC | GCATCTACCCCAGTTTTAACTTCTA |
| | (SEQ ID NO: 23) | TACTAAT | (SEQ ID NO: 25) |
| | | (SEQ ID NO: 24) | |
| 16 | CCAACCAAAAGAAAAGGAGAGT | CAGCTCCCAAAAAGCCAAAGGAACC | GCATCTACCCCAGTTTTAACTTCTA |
| | (SEQ ID NO: 26) | (SEQ ID NO: 27) | (SEQ ID NO: 25) |
| 17 | GGGGCAGCTCCCAAAAAG | AAGGAACCCGTGCAAGTGCCAAAAC | ATCTGGATCCCCCATTTCTG |
| | (SEQ ID NO: 28) | TACTAATAAA | (SEQ ID NO: 29) |
| | | (SEQ ID NO: 18) | |
| 18 | GGGCAGCTCCCAAAAAGC | AAAGGAACCCGTGCAAGTGCCAAAA | CATCTGGATCCCCCATTTCTG |
| | (SEQ ID NO: 15) | CTACTAATAAA | (SEQ ID NO: 30) |
| | | (SEQ ID NO: 14) | |
| 19 | GGGGCAGCTCCCAAAAAG | AAGGAACCCGTGCAAGTGCCAAAAC | TCATCTGGATCCCCCATTTCT |
| | (SEQ ID NO: 28) | TACTAATAAA | (SEQ ID NO: 31) |
| | | (SEQ ID NO: 18) | |
| 20 | GGGCAGCTCCCAAAAAGC | AAAGGAACCCGTGCAAGTGCCAAAA | TTAGATTTCCACAGGTTAGGTCCTCA |
| | (SEQ ID NO: 25) | CTACTAATAAA | TT |
| | | (SEQ ID NO: 14) | (SEQ ID NO: 32) |
| 21 | CAGGGGCAGCTCCCAAAA | AAAGGAACCCGTGCAAGTGCCAAAA | TAGATTTCCACAGGTTAGGTCCTCA |
| | (SEQ ID NO: 20) | CTACTAATAAA | TT |
| | | (SEQ ID NO: 14) | (SEQ ID NO: 32) |
| 22 | AGGGGCAGCTCCCAAAAAG | CAAAGGAACCCGTGCAAGTGCCAAA | GTAGATTTCCACAGGTTAGGTCCTC |
| | (SEQ ID NO: 10) | ACTACTAATAAA | ATT |
| | | (SEQ ID NO: 11) | (SEQ ID NO: 33) |
| 23 | AGGGGCAGCTCCCAAAAAG | CAAAGGAACCCGTGCAAGTGCCAAA | AGTAGATTTCCACAGGTTAGGTCCT |
| | (SEQ ID NO: 10) | ACTACTAATAAA | CATT |
| | | (SEQ ID NO: 11) | (SEQ ID NO: 34) |
| 24 | AGGGGCAGCTCCCAAAA | AAGGAACCCGTGCAAGTGCCAAAAC | GGGAATTCTTGCTGTGCTGTA |
| | (SEQ ID NO: 23) | TACTAAT | (SEQ ID NO: 35) |
| | | (SEQ ID NO: 24) | |
| 25 | GGGGCAGCTCCCAAAAAG | AAGGAACCCGTGCAAGTGCCAAAAC | TTCATCTGGATCCCCCATTTCT |
| | (SEQ ID NO: 28) | TACTAATAAA | (SEQ ID NO: 36) |
| | | (SEQ ID NO: 18) | |
| 26 | CCAACCAAAAGAAAAGGAGAGT | CAGCTCCCAAAAAGCCAAAGGAACC | CATCTGGATCCCCCATTTCT |
| | GT | C | (SEQ ID NO: 39) |
| | (SEQ ID NO: 37) | (SEQ ID NO: 38) | |
| 27 | CCAACCAAAAGAAAAGGAGAGTG | CAGCTCCCAAAAAGCCAAAGGAACC | TTCATCTGGATCCCCCATTTCT |
| | T | C | (SEQ ID NO: 36) |
| | (SEQ ID NO: 37) | (SEQ ID NO: 38) | |
| 28 | GGGGCAGCTCCCAAAAAG | AAGGAACCCGTGCAAGTGCCAAAAC | GTAGATTTCCACAGGTTAGGTCCTC |
| | (SEQ ID NO: 28) | TACTAATAAAAG | ATT |
| | | (SEQ ID NO: 40) | (SEQ ID NO: 33) |
| 29 | CAGGGGCAGCTCCCAAA | AAAGGAACCCGTGCAAGTGCCAAAA | TTAGATTTCCACAGGTTAGGTCCTCA |
| | (SEQ ID NO: 13) | CTACTAATAAAAG | TT |
| | | (SEQ ID NO: 41) | (SEQ ID NO: 32) |
| 30 | AGGGGCAGCTCCCAAAAA | AAAGGAACCCGTGCAAGTGCCAAAA | GGGAATTCTTGCTGTGCTGTAA |
| | (SEQ ID NO: 42) | CTACTAATAAA | (SEQ ID NO: 43) |
| | | (SEQ ID NO: 14) | |
| 31 | GGGGCAGCTCCCAAAAAG | AAGGAACCCGTGCAAGTGCCAAAAC | GGGGAATTCTTGCTGTGCTGTA |
| | (SEQ ID NO: 28) | TACTAATAAA | (SEQ ID NO: 44) |
| | | (SEQ ID NO: 18) | |
| 32 | AGGGGCAGCTCCCAAAAAG | CAAAGGAACCCGTGCAAGTGCCAAA | GGGAATTCTTGCTGTGCTGTAAC |
| | (SEQ ID NO: 10) | ACTACTAATAAA | (SEQ ID NO: 45) |
| | | (SEQ ID NO: 11) | |
| 33 | GGGCAGCTCCCAAAAAGC | AAAGGAACCCGTGCAAGTGCCAAAA | AGGGGAATTCTTGCTGTGCTGTA |
| | (SEQ ID NO: 15) | CTACTAATAAA | (SEQ ID NO: 46) |
| | | (SEQ ID NO: 14) | |
| 34 | AGGGGCAGCTCCCAAAAAG | CAAAGGAACCCGTGCAAGTGCCAAA | GGGGAATTCTTGCTGTGCTGTAA |
| | (SEQ ID NO: 10) | ACTACTAATAAA | (SEQ ID NO: 47) |
| | | (SEQ ID NO: 11) | |
| 35 | CAGGGGCAGCTCCCAAAA | CAAAGGAACCCGTGCAAGTGCCAAA | GGGGAATTCTTGCTGTGCTGTAAC |
| | (SEQ ID NO: 20) | ACTACTAATAAA | (SEQ ID NO: 48) |
| | | (SEQ ID NO: 11) | |
| 36 | AGGGGCAGCTCCCAAAAAG | CAAAGGAACCCGTGCAAGTGCCAAA | AGGGGAATTCTTGCTGTGCTGTAA |
| | (SEQ ID NO: 10) | ACTACTAATAAA | (SEQ ID NO: 49) |
| | | (SEQ ID NO: 11) | |
| 37 | GGGCAGCTCCCAAAAAGC | AAAGGAACCCGTGCAAGTGCCAAAA | TTTCATCTGGATCCCCCATTTC |
| | (SEQ ID NO: 15) | CTACTAATAAA | (SEQ ID NO: 50) |
| | | (SEQ ID NO: 14) | |
| 38 | AGGGGCAGCTCCCAAAAAG | CAAAGGAACCCGTGCAAGTGCCAAA | TTTCATCTGGATCCCCCATTTCT |
| | (SEQ ID NO: 10) | ACTACTAATAAA | (SEQ ID NO: 51) |
| | | (SEQ ID NO: 11) | |
| 39 | GGGGCAGCTCCCAAAA | AAGGAACCCGTGCAAGTGCCAAAAC | GGGAGGGGAATTCTTTGCT |
| | (SEQ ID NO: 52) | (SEQ ID NO: 53) | (SEQ ID NO: 54) |
| 40 | CAGGGGCAGCTCCCAAAA | CAAAGGAACCCGTGCAAGTGCCAAA | GGGGAATTCTTGCTGTGCTGTAA |
| | (SEQ ID NO: 20) | ACTACTAATAAA | (SEQ ID NO: 47) |
| | | (SEQ ID NO: 11) | |
| 41 | AGGGGCAGCTCCCAAAAAG | CAAAGGAACCCGTGCAAGTGCCAAA | AGGGGAATTCTTGCTGTGCTGTAA |
| | (SEQ ID NO: 10) | ACTACTAATAAA | (SEQ ID NO: 49) |
| | | (SEQ ID NO: 11) | |
| 42 | CCCAACCAAAAGAAAAGGAGAGT | CAAAGGAACCCGTGCAAGTGCCAAA | ATTTCCACAGGTTAGGTCCTCAT |
| | (SEQ ID NO: 55) | A | (SEQ ID NO: 57) |
| | | (SEQ ID NO: 56) | |
| 43 | CCCCAACCAAAAGAAAAGGAGAG | CAAAGGAACCCGTGCAAGTGCCAAA | GTAGATTTCCACAGGTTAGGTCCTC |
| | T | ACTACTA | AT |
| | (SEQ ID NO: 58) | (SEQ ID NO: 59) | (SEQ ID NO: 19) |
| 44 | CCCAACCAAAAGAAAAGGAGAGT | CAAAGGAACCCGTGCAAGTGCCAAA | AGTAGATTTCCACAGGTTAGGTCCT |
| | GT | ACTACTAAT | CAT |
| | (SEQ ID NO: 60) | (SEQ ID NO: 61) | (SEQ ID NO: 22) |
| 45 | CCCAACCAAAAGAAAAGGAGAGT | AAAGGAACCCGTGCAAGTGCCAAAA | AGATTTCCACAGGTTAGGTCCTCAT |
| | (SEQ ID NO: 55) | CTACTAATAAA | T |
| | | (SEQ ID NO: 14) | (SEQ ID NO: 62) |
| 46 | CCCAACCAAAAGAAAAGGAGAGT | TCCCAAAAAGCCAAAGGAACCCGTG | AGATTTCCACAGGTTAGGTCCTCAT |
| | (SEQ ID NO: 55) | (SEQ ID NO: 63) | T |
| | | | (SEQ ID NO: 62) |
| 47 | CCCAACCAAAAGAAAAGGAGAGT | AGCTCCCAAAAAGCCAAAGGAACCC | AGATTTCCACAGGTTAGGTCCTCAT |
| | (SEQ ID NO: 55) | G | T |
| | | (SEQ ID NO: 64) | (SEQ ID NO: 62) |
| 48 | CCCCAACCAAAAGAAAAGGAGAG | AGCTCCCAAAAAGCCAAAGGAACCC | AGTAGATTTCCACAGGTTAGGTCCT |
| | (SEQ ID NO: 58) | GT | CATT |
| | | (SEQ ID NO: 65) | (SEQ ID NO: 34) |
| 49 | CCAACCAAAAGAAAAGGAGAGT | CAGCTCCCAAAAAGCCAAAGGAACC | TCTGTTATAGCATCTACCCCAGTT |
| | (SEQ ID NO: 26) | (SEQ ID NO: 27) | (SEQ ID NO: 66) |
| 50 | CCAACCAAAAGAAAAGGAGAGTG | CAGCTCCCAAAAAGCCAAAGGAACC | AGTTACAGCCTCCCACATCAGTAGA |
| | (SEQ ID NO: 37) | C | TT |
| | | (SEQ ID NO: 38) | (SEQ ID NO: 67) |
| 51 | CAGGGGCAGCTCCCAAAAA | CAAAGGAACCCGTGCAAGTGCCCAAA | TTTCATCTGGATCCCCCATTTC |
| | (SEQ ID NO: 68) | ACT | (SEQ ID NO: 50) |
| | | (SEQ ID NO: 69) | |
| 52 | CAGGGGCAGCTCCCAAAA | AAAGGAACCCGTGCAAGTGCCAAAA | TCATCTGGATCCCCCATTTCTG |
| | (SEQ ID NO: 20) | CTACTAATAAAAG | (SEQ ID NO: 70) |
| | | (SEQ ID NO: 41) | |
| 53 | AGGGGCAGCTCCCAAAAAG | AAGGAACCCGTGCAAGTGCCAAAAC | GTTACAGCCTCCCACATCAGTAGAT |
| | (SEQ ID NO: 10) | TACTAATAAAA | T |
| | | (SEQ ID NO: 71) | (SEQ ID NO: 72) |
| 54 | CCCAACCAAAAGAAAAGGAGAGT | CAGCTCCCAAAAAGCCAAAGGAACC | CCTCTGTTATAGCATCTACCCCAGT |
| | (SEQ ID NO: 55) | C | TT |
| | | (SEQ ID NO: 38) | (SEQ ID NO: 73) |
| 55 | CCCAACCAAAAGAAAAGGAGAGT | CAGCTCCCAAAAAGCCAAAGGAACC | ACCTCTGTTATAGCATCTACCCCAG |
| | (SEQ ID NO: 55) | C | TT |
| | | (SEQ ID NO: 38) | (SEQ ID NO: 74) |
| 56 | CCAACCAAAAGAAAAGGAGAGTG | CAGCTCCCAAAAAGCCAAAGGAACC | ACCTCTGTTATAGCATCTACCCCAG |
| | T | C | TTT |
| | (SEQ ID NO: 37) | (SEQ ID NO: 38) | (SEQ ID NO: 75) |
| 57 | GCAGCTCCCAAAAAGC | CTCCTTTTATTAGTAGTTTTGGCAC | GCATCTACCCCAGTTTTAACTTCTA |
| | (SEQ ID NO: 76) | TTGCACGG | (SEQ ID NO: 25) |
| | | (SEQ ID NO: 77) | |
| 58 | CCCAACCAAAAGAAAAGGAGAGT | CAGCTCCCAAAAAGCCAAAGGAACC | TCCTTTTATTAGTAGTTTTGGCACT |
| | (SEQ ID NO: 55) | C | TG |
| | | (SEQ ID NO: 38) | (SEQ ID NO: 78) |
| 59 | CCAACCAAAAGAAAAGGAGAGT | CAGCTCCCAAAAAGCCAAAGGAACC | CCTTTTTATTAGTAGTTTTGGCACTT |
| | (SEQ ID NO: 26) | (SEQ ID NO: 27) | G |
| | | | (SEQ ID NO: 79) |
| 60 | CAACCAAAAGAAAAGGAGAGTGT | AAGGAACCCGTGCAAGTGCCAAA | TCATCTGGATCCCCCATTT |
| | (SEQ ID NO: 80) | (SEQ ID NO: 81) | (SEQ ID NO: 82) |
| 61 | CCCCAACCAAAAGAAAAGGAGAG | AAAGGAACCCGTGCAAGTGCCAAAA | TACCTCTGTTATAGCATCTACCCCA |
| | T | CTACTAATAA | GTTTT |
| | (SEQ ID NO: 58) | (SEQ ID NO: 83) | (SEQ ID NO: 84) |
| 62 | GGCAGCTCCCAAAAAGC | CTCCTTTTATTAGTAGTTTTGGCAC | ATCTGGATCCCCCATTTCTG |
| | (SEQ ID NO: 76) | TTGCACGG | (SEQ ID NO: 29) |
| | | (SEQ ID NO: 77) | |
| 63 | GGGCAGCTCCCAAAAAGC | CCCGTGCAAGTGCCAAAACTACTAA | AGATTTCCACAGGTTAGGTCCTCAT |
| | (SEQ ID NO: 15) | TAAAAGGA | (SEQ ID NO: 21) |
| | | (SEQ ID NO: 85) | |
| 64 | AGGGGCAGCTCCCAAAA | TAAGGTTTTCATCTGGATCCCCCAT | GGGAATTCTTGCTGTGCTGTA |
| | (SEQ ID NO: 23) | TTCTG | (SEQ ID NO: 35) |
| | | (SEQ ID NO: 86) | |
| 65 | CTCCCAAAAAGCCAAAGGA | CTCCTTTTATTAGTAGTTTTGGCAC | GCATCTACCCCAGTTTTAACTTCTA |
| | (SEQ ID NO: 87) | TTGCACGG | (SEQ ID NO: 25) |
| | | (SEQ ID NO: 77) | |
| 66 | AGCTCCCAAAAAGCCAAAG | CTCCTTTTATTAGTAGTTTTGGCAC | GCATCTACCCCAGTTTTAACTTCTA |
| | (SEQ ID NO: 88) | TTGCACGG | (SEQ ID NO: 25) |
| | | (SEQ ID NO: 77) | |
| 67 | AGGGGCAGCTCCCAAAAAG | CAAAGGAACCCGTGCAAGTGCCAAA | TTTTCATCTGGATCCCCCATTTC |
| | (SEQ ID NO: 10) | ACTACTAATAAA | (SEQ ID NO: 89) |
| | | (SEQ ID NO: 11) | |
| 68 | AGGGGCAGCTCCCAAAAAG | CAAAGGAACCCGTGCAAGTGCCAAA | ACTAAAGCCCCTAAGGTTTTCATCT |
| | (SEQ ID NO: 10) | ACTACTAATAAA | G |
| | | (SEQ ID NO: 11) | (SEQ ID NO: 90) |
| 69 | CAGGGGCAGCTCCCAAAA | CAAAGGAACCCGTGCAAGTGCCAAA | GACTAAAGCCCCTAAGGTTTTCATC |
| | (SEQ ID NO: 20) | ACTACTAATAAA | TG |
| | | (SEQ ID NO: 11) | (SEQ ID NO: 91) |
| 70 | CCCAACCAAAAGAAAAGGAGAGT | TCCCAAAAAGCCAAAGGAACCCGTG | TCCTTTTTATTAGTAGTTTTGGCACT |
| | (SEQ ID NO: 55) | (SEQ ID NO: 63) | TG |
| | | | (SEQ ID NO: 78) |
| 71 | ATGAAAACCTTAGGGGCTTTAGT | CCCTGTTACAGCACAGCAAGAATTC | TTTCCACAGGTTAGGTCCTCAT |
| | C | CC | (SEQ ID NO: 94) |
| | (SEQ ID NO: 92) | (SEQ ID NO: 93) | |
| 72 | TGAAAACCTTAGGGGCTTTAGTC | CCTGTTACAGCACAGCAAGAATTCC | ATTTCCACAGGTTAGGTCCTCAT |
| | (SEQ ID NO: 95) | CCT | (SEQ ID NO: 57) |
| | | (SEQ ID NO: 96) | |
| 73 | ATGAAAACCTTAGGGGCTTTAGT | CTTCCCTGTTACAGCACAGCAAGAA | TTTCCACAGGTTAGGTCCTCAT |
| | CT | TTCCC | (SEQ ID NO: 94) |
| | (SEQ ID NO: 97) | (SEQ ID NO: 134) | |
| 74 | ATGAAAACCTTAGGGGCTTTAGT | CCCTGTTACAGCACAGCAAGAATTC | TTTCCACAGGTTAGGTCCTCAT |
| | CT | CCCT | (SEQ ID NO: 94) |
| | (SEQ ID NO: 97) | (SEQ ID NO: 99) | |
| 75 | GAAAACCTTAGGGGCTTTAGTCT | CCTGTTACAAGCACAGCAAGAATTCC | TTTCCACAGGTTAGGTCCTCAT |
| | (SEQ ID NO: 100) | CC | (SEQ ID NO: 94) |
| | | (SEQ ID NO: 101) | |
| 76 | ATGAAAACCTTAGGGGCTTTAGT | TCCCTGTTACAGCACAGCAAGAATT | TTTCCACAGGTTAGGTCCTCATT |
| | CT | CCC | (SEQ ID NO: 103) |
| | (SEQ ID NO: 97) | (SEQ ID NO: 102) | |
| 77 | TGAAAACCTTAGGGGCTTTAGTC | CCTGTTACAGCACAGCAAGAATTCC | GATTTCCACAGGTTAGGTCCTCAT |
| | (SEQ ID NO: 95) | CCT | (SEQ ID NO: 12) |
| | | (SEQ ID NO: 96) | |
| 78 | GATGAAAACCTTAGGGGCTTTAG | CCTGTTACAGCACAGCAAGAATTCC | TTTCCACAGGTTAGGTCCTCAT |
| | T | CCT | (SEQ ID NO: 94) |
| | (SEQ ID NO: 104) | (SEQ ID NO: 96) | |
| 79 | ATGAAAACCTTAGGGGCTTTAGT | CCTGTTACAGCACAGCAAGAATTCC | ATTTCCACAGGTTAGGTCCTCATT |
| | CT | CCT | (SEQ ID NO: 105) |
| | (SEQ ID NO: 97) | (SEQ ID NO: 96) | |
| 80 | ATGAAAACCTTAGGGGCTTTAGT | CCTGTTACAGCACAGCAAGAATTCC | AGATTTCCACAGGTTAGGTCCTCAT |
| | CT | CCT | (SEQ ID NO: 21) |
| | (SEQ ID NO: 97) | (SEQ ID NO: 96) | |
| 81 | AGATGAAAACCTTAGGGGCTTTA | CCTGTTACAGCACAGCAAGAATTCC | TTTCCACAGGTTAGGTCCTCAT |
| | GT | CCT | (SEQ ID NO: 94) |
| | (SEQ ID NO: 106) | (SEQ ID NO: 96) | |
| 82 | GATGAAAACCTTAGGGGCTTTAG | CTTCCCTGTTACAGCACAGCAAGAA | TTTCCACAGGTTAGGTCCTCATT |
| | TCT | TTCCCC | (SEQ ID NO: 94) |
| | (SEQ ID NO: 107) | (SEQ ID NO: 108) | |
| 83 | CAGATGAAAACCTTAGGGGCTTT | TTCCCTGTTACAGCACAGCAAGAAT | TTTCCACAGGTTAGGTCCTCATT |
| | AG | TCCCCT | (SEQ ID NO: 103) |
| | (SEQ ID NO: 109) | (SEQ ID NO: 110) | |
| 84 | TGAAAACCTTAGGGGCTTTAGTC | CCTGTTACAGCACAGCAAGAATTCC | GATTTCCACAGGTTAGGTCCTCATT |
| | T | CCT | (SEQ ID NO: 112) |
| | (SEQ ID NO: 111) | (SEQ ID NO: 96) | |
| 85 | AGAAATGGGGGATCCAGATG | CCTGTTACAGCACAGCAAGAATTCC | TTTCCACAGGTTAGGTCCTCAT |
| | (SEQ ID NO: 113) | CCT | (SEQ ID NO: 94) |
| | | (SEQ ID NO: 96) | |
| 86 | ATGGGGGATCCAGATGAAAAC | CCTGTTACAGCACAGCAAGAATTCC | TTTCCACAGGTTAGGTCCTCATT |
| | (SEQ ID NO: 114) | CCT | (SEQ ID NO: 103) |
| | | (SEQ ID NO: 96) | |
| 87 | GAAATGGGGGATCCAGATGA | CTTCCCTGTTACAGCACAGCAAGAA | TTCCACAGGTTAGGTCCTCATT |
| | (SEQ ID NO: 115) | TTCCC | (SEQ ID NO: 116) |
| | | (SEQ ID NO: 134) | |
| 88 | AAATGGGGGATCCAGATGAA | CCTGTTACAGCACAGCAAGAATTCC | TTTCCACAGGTTAGGTCCTCAT |
| | (SEQ ID NO: 117) | CCT | (SEQ ID NO: 94) |
| | | (SEQ ID NO: 96) | |
| 89 | CAGATGAAAACCTTAGGGGCTTT | CTTCCCTGTTACAGCACAGCAAGAA | ATTTCCACAGGTTAGGTCCTCATT |
| | AG | TTCCCC | (SEQ ID NO: 105) |
| | (SEQ ID NO: 109) | (SEQ ID NO: 108) | |
| 90 | AATGGGGGATCCAGATGAAA | CCTGTTACAGCACAGCAAGAATTCC | TTTCCACAGGTTAGGTCCTCAT |
| | (SEQ ID NO: 118) | CCT | (SEQ ID NO: 94) |
| | | (SEQ ID NO: 96) | |
| 91 | TGGGGGATCCAGATGAAAAC | CCTGTTACAGCACAGCAAGAATTCC | TTTCCACAGGTTAGGTCCTCATT |
| | (SEQ ID NO: 119) | CCT | (SEQ ID NO: 103) |
| | | (SEQ ID NO: 96) | |
| 92 | GAAATGGGGGATCCAGATGAA | CTTCCCTGTTACAGCACAGCAAGAA | TTTCCACAGGTTAGGTCCTCATT |
| | (SEQ ID NO: 120) | TTCCCC | (SEQ ID NO: 103) |
| | | (SEQ ID NO: 108) | |
| 93 | AAATGGGGGATCCAGATGAAA | TCCCTGTTACAGCACAGCAAGAATT | TTTCCACAGGTTAGGTCCTCATT |
| | (SEQ ID NO: 121) | CCCCT | (SEQ ID NO: 103) |
| | | (SEQ ID NO: 122) | |
| 94 | AGAAATGGGGGATCCAGATGA | TTCCCTGTTACAGCACAGCAAGAAT | ATTTCCACAGGTTAGGTCCTCATT |
| | (SEQ ID NO: 123) | TCCCC | (SEQ ID NO: 105) |
| | | (SEQ ID NO: 124) | |
| 95 | AATGGGGGATCCAGATGAAAA | CTTCCCTGTTACAGCACAGCAAGAA | TTTCCACAGGTTAGGTCCTCATT |
| | (SEQ ID NO: 125) | TTCCC | (SEQ ID NO: 103) |
| | | (SEQ ID NO: 134) | |
| 96 | CAGAAATGGGGGATCCAGATG | CTTCCCTGTTACAGCACAGCAAGAA | GATTTCCACAGGTTAGGTCCTCAT |
| | (SEQ ID NO: 126) | TTCCCCT | (SEQ ID NO: 12) |
| | | (SEQ ID NO: 127) | |
| 97 | CAGATGAAAACCTTAGGGGCTTT | TTCCCTGTTACAGCACAGCAAGAAT | AGATTTCCACAGGTTAGGTCCTCAT |
| | AGT | TCCCCT | T |
| | (SEQ ID NO: 128) | (SEQ ID NO: 110) | (SEQ ID NO: 62) |
| 98 | AATGGGGGATCCAGATGAAAAC | CTTCCCTGTTACAGCACAGCAAGAA | ATTTCCACAGGTTAGGTCCTCATT |
| | (SEQ ID NO: 129) | TTCCCCT | (SEQ ID NO: 105) |
| | | (SEQ ID NO: 127) | |
| 99 | AGAAATGGGGGATCCAGATGAA | CTTCCCTGTTACAGCACAGCAAGAA | GATTTCCACAGGTTAGGTCCTCAT |
| | (SEQ ID NO: 130) | TTCCCCT | (SEQ ID NO: 12) |
| | | (SEQ ID NO: 127) | |
| 100 | GCTTCCTAAACCCAGAAATGG | CCTGTTACAGCACAGCAAGAATTCC | TTTCCACAGGTTAGGTCCTCAT |
| | (SEQ ID NO: 131) | CCT | (SEQ ID NO: 94) |
| | | (SEQ ID NO: 96) | |
| 101 | ATGCTTCCTAAACCCAGAAATG | CCCTGTTACAGCACAGCAAGAATTC | TTTCCACAGGTTAGGTCCTCAT |
| | (SEQ ID NO: 132) | CC | (SEQ ID NO: 94) |
| | | (SEQ ID NO: 93) | |
| 102 | GATGAAAACCTTAGGGGCTTTAG | TGTTACAGCACAGCAAGAATTCCCC | GATTTCCACAGGTTAGGTCCTCAT |
| | TCT | TCC | (SEQ ID NO: 12) |
| | (SEQ ID NO: 107) | (SEQ ID NO: 133) | |
| 103 | ATGAAAACCTTAGGGGCTTTAGT | CTTCCCTGTTACAGCACAGCAAGAA | ATTTCCACAGGTTAGGTCCTCAT |
| | CT | TTCCC | (SEQ ID NO: 57) |
| | (SEQ ID NO: 97) | (SEQ ID NO: 134) | |
| 104 | CAGTGATAGCCCAGACAGAAAAA | CTTCCCTGTTACAGCACAGCAAGAA | ATTTCCACAGGTTAGGTCCTCAT |
| | (SEQ ID NO: 98) | TTCCC | (SEQ ID NO: 57) |
| | | (SEQ ID NO: 134) | |
| 105 | ATGAAAACCTTAGGGGCTTTAGT | CCAGCCTTGATCGTTACAAAGGACG | ATTTCCACAGGTTAGGTCCTCAT |
| | CT | ATGC | (SEQ ID NO: 57) |
| | (SEQ ID NO: 97) | (SEQ ID NO: 135) | |

A PCR primer pair for amplifying BK virus DNA can comprise any one of the following pairs of primer sequences: (1) SEQ ID NOS: 1 and 3; (2) SEQ ID NOS: 6 and 3; (3) SEQ ID NOS: 7 and 3; (4) SEQ ID NOS: 8 and 3; (5) SEQ ID NOS: 9 and 3; (6) SEQ ID NOS: 10 and 12; (7) SEQ ID NOS: 13 and 12; (8) SEQ ID NOS: 15 and 17; (9) SEQ ID NOS: 15 and 19; (10) SEQ ID NOS: 20 and 21; (I 1) SEQ ID NOS: 15 and 22; (12) SEQ ID NOS: 23 and 25; (13) SEQ ID NOS: 26 and 25; (14) SEQ ID NOS: 28 and 29; (15) SEQ ID NOS: 15 and 30; (16) SEQ ID NOS: 28 and 31; (17) SEQ ID NOS: 15 and 32; (18) SEQ ID NOS: 20 and 32; (19) SEQ ID NOS: 10 and 33; (20) SEQ ID NOS: 10 and 34; (21) SEQ ID NOS: 23 and 35; (22) SEQ ID NOS: 28 and 36; (23) SEQ ID NOS: 37 and 39; (24) SEQ ID NOS: 37 and 36; (25) SEQ ID NOS: 28 and 33; (26) SEQ ID NOS: 13 and 32; (27) SEQ ID NOS: 42 and 43; (28) SEQ ID NOS: 28 and 44; (29) SEQ ID NOS: 10 and 45; (30) SEQ ID NOS: 5 and 46; (31) SEQ ID NOS: 10 and 47; (32) SEQ ID NOS: 20 and 48; (33) SEQ ID NOS: 10 and 49; (34) SEQ ID NOS: 15 and 50; (35) SEQ ID NOS: 10 and 51; (36) SEQ ID NOS: 52 and 54; (37) SEQ ID NOS: 20 and 47; (38) SEQ ID NOS: 10 and 49; (39) SEQ ID NOS: 55 and 57; (40) SEQ ID NOS: 58 and 19; (41) SEQ ID NOS: 60 and 22; (42) SEQ ID NOS: 55 and 62; (43) SEQ ID NOS: 58 and 34; (44) SEQ ID NOS: 26 and 66; (45) SEQ ID NOS: 37 and 67; (46) SEQ ID NOS: 68 and 50; (47) SEQ ID NOS: 20 and 70; (48) SEQ ID NOS: 10 and 72; (49) SEQ ID NOS: 55 and 73; (50) SEQ ID NOS: 55 and 74; (51) SEQ ID NOS: 37 and 75; (52) SEQ ID NOS: 76 and 25; (53) SEQ ID NOS: 55 and 78, (54) SEQ ID NOS: 26 and 79; (55) SEQ ID NOS: 80 and 82; (56) SEQ ID NOS: 58 and 84; (57) SEQ ID NOS: 76 and 29; (58) SEQ ID NOS: 15 and 21; (59) SEQ ID NOS: 23 and 35; (60) SEQ ID NOS: 87 and 25; (61) SEQ ID NOS: 88 and 25; (62) SEQ ID NOS: 10 and 89; (63) SEQ ID NOS: 10 and 90; (64) SEQ ID NOS: 20 and 91; (65) SEQ ID NOS: 55 and 78; (66) SEQ ID NOS: 92 and 94; (67) SEQ ID NOS: 95 and 57; (68) SEQ ID NOS: 97 and 94; (69) SEQ ID NOS: 100 and 94; (70) SEQ ID NOS: 97 and 103; (71) SEQ ID NOS: 95 and 12; (72) SEQ ID NOS: 104 and 94; (73) SEQ ID NOS: 97 and 105; (74) SEQ ID NOS: 97 and 21; (75) SEQ ID NOS: 106 and 94; (76) SEQ ID NOS: 107 and 103; (77) SEQ ID NOS: 109 and 103; (78) SEQ ID NOS: 111 and 112; (79) SEQ ID NOS: 113 and 94; (80) SEQ ID NOS: 114 and 103; (81) SEQ ID NOS: 115 and 116; (82) SEQ ID NOS: 117 and 94; (83) SEQ ID NOS: 109 and 105; (84) SEQ ID NOS: 118 and 94: (85) SEQ ID NOS: 119 and 103; (86) SEQ ID NOS: 120 and 103; (87) SEQ ID NOS: 121 and 103; (88) SEQ ID NOS: 123 and 105; (89) SEQ ID NOS: 125 and 103; (90) SEQ ID NOS: 126 and 12; (91) SEQ ID NOS: 128 and 62; (92) SEQ ID NOS: 129 and 105; (93) SEQ ID NOS: 130 and 12; (94) SEQ ID NOS: 131 and 94; (95) SEQ ID NOS: 132 and 94; (96) SEQ ID NOS: 107 and 12; (97) SEQ ID) NOS: 97 and 57; and (98) SEQ ID NOS: 98 and 57 Any pair of primers can be used simultaneously in a multiplex PCR reaction with one or more other primer pairs, so that multiple amplicons are amplified simultaneously. For example, SEQ ID NOS: 97 and 98 can be used in a multiplex assay along with SEQ ID NO:57 as a reverse primer and SEQ ID NO:134 as a probe.

The preceding numbering of the ninety-eight pairs of primers does not correspond exactly to the “Group” numbering scheme in Table 2 because certain groups use the same primer pair, but different internal probes. For example, Groups 1, 2, and 3 of Table 2 each employ the forward primer of SEQ ID NO:1 and the reverse primer of SEQ ID NO:3, but three different internal probes in each instance, e.g., SEQ ID NOS: 2, 4, and 5. Accordingly, primer pair “(1)” of the preceding passage implies any one of Groups 1, 2, and 3 of Table 2. Likewise, Groups 7 and 8 of Table 2 each employ the primers of SEQ ID NOS: 9 and 3, but one Group uses the probe of SEQ ID NO:2 while the other Group uses the probe of SEQ ID NO:4. Groups 7 and 8 are depicted in primer pair “(5)” in the preceding passage.

The probe of SEQ ID NO:2 can be used to hybridize to the product amplified with the primer pair of Groups 1, 4, and 7 depicted in Table 2. The probe of SEQ ID NO:4 can be used to hybridize to the product amplified with the primer pair of Groups 2, 6, and 8 depicted in Table 2. The probe of SEQ ID NO:5 can be used to hybridize to the product amplified with the primer pair of Groups 3 and 5 depicted in Table 2. The probe of SEQ ID NO:11 can be used to hybridize to the product amplified with the primer pair of Groups 9, 22, 23 ,32, 34, 35, 36, 38, 40, 41, 67, 68, and 69 depicted in Table 2. The probe of SEQ ID NO: 14 can be used to hybridize to the product amplified with the primer pair of Groups 10, 14, 18, 20, 21, 30, 33, 37, and 45 depicted in Table 2. The probe of SEQ ID NO:16 can be used to hybridize to the product amplified with the primer pair of Group 11 depicted in Table 2. The probe of SEQ ID NO:18 can be used to hybridize to the product amplified with the primer pair of Groups 12, 13, 17, 19, 25, and 31 depicted in Table 2. The probe of SEQ ID NO:24 can be used to hybridize to the product amplified with the primer pair of Groups 15 and 24 depicted in Table 2. The probe of SEQ ID NO:27 can be used to hybridize to the product amplified with the primer pair of Groups 16, 49, 59 depicted in Table 2. The probe of SEQ ID NO:38 can be used to hybridize to the product amplified with the primer pair of Groups 26, 27, 50, 54, 55, 56, and 58 depicted in Table 2. The probe of SEQ ID NO:40 can be used to hybridize to the product amplified with the primer pair of Group 28 depicted in Table 2. The probe of SEQ ID NO:41 can be used to hybridize to the product amplified with the primer pair of Groups 29 and 52 depicted in Table 2. The probe of SEQ ID NO:53 can be used to hybridize to the product amplified with the primer pair of Group 39 depicted in Table 2. The probe of SEQ ID NO:56 can be used to hybridize to the product amplified with the primer pair of Group 42 depicted in Table 2. The probe of SEQ ID NO:59 can be used to hybridize to the product amplified with the primer pair of Group 43 depicted in Table 2. The probe of SEQ ID NO:61 can be used to hybridize to the product amplified with the primer pair of Group 44 depicted in Table 2. The probe of SEQ ID NO:63 can be used to hybridize to the product amplified with the primer pair of Groups 46 and 70 depicted in Table 2. The probe of SEQ ID NO:64 can be used to hybridize to the product amplified with the primer pair of Group 47 depicted in Table 2. The probe of SEQ ID) NO:65 can be used to hybridize to the product amplified with the primer pair of Group 48 depicted in Table 2. The probe of SEQ ID NO:69 can be used to hybridize to the product amplified with the primer pair of Group 51 depicted in Table 2 The probe of SEQ ID NO:71 can be used to hybridize to the product amplified with the primer pair of Group 53 depicted in Table 2. The probe of SEQ ID NO:77 can be used to hybridize to the product amplified with the primer pair of Groups 57, 62, 65, and 66 depicted in Table 2. The probe of SEQ ID NO:81 can be used to hybridize to the product amplified with the primer pair of Group 60 depicted in Table 2. The probe of SEQ ID NO:83 can be used to hybridize to the product amplified with the primer pair of Group 61 depicted in Table 2. The probe of SEQ ID NO:85 can be used to hybridize to the product amplified with the primer pair of Group 63 depicted in Table 2. The probe of SEQ ID NO:86 can be used to hybridize to the product amplified with the primer pair of Group 64 depicted in Table 2. The probe of SEQ ID NO:93 can be used to hybridize to the product amplified with the primer pair of Groups 71 and 101 depicted in Table 2. The probe of SEQ ID NO:96 can be used to hybridize to the product amplified with the primer pair of Groups 72, 77, 78, 79, 80, 81, 84, 85, 86, 88, 90, 91, and 101 depicted in Table 2. The probe of SEQ ID NO:99 can be used to hybridize to the product amplified with the primer pair of Group 74 depicted in Table 2. The probe of SEQ ID NO:101 can be used to hybridize to the product amplified with the primer pair of Group 75 depicted in Table 2. The probe of SEQ ID NO:102 can be used to hybridize to the product amplified with the primer pair of Group 76 depicted in Table 2. The probe of SEQ ID NO:108 can be used to hybridize to the product amplified with the primer pair of Groups 82, 89, and 92 depicted in Table 2. The probe of SEQ ID NO:110 can be used to hybridize to the product amplified with the primer pair of Groups 83 and 97 depicted in Table 2. The probe of SEQ ID NO:122 can be used to hybridize to the product amplified with the primer pair of Group 93 depicted in Table 2. The probe of SEQ ID NO:124 can be used to hybridize to the product amplified with the primer pair of Group 94 depicted in Table 2. The probe of SEQ ID NO:127 can be used to hybridize to the product amplified with the primer pair of Groups 96, 98, and 99 depicted in Table 2. The probe of SEQ ID NO:133 can be used to hybridize to the product amplified with the primer pair of Group 102 depicted in Table 2. The probe of SEQ ID NO:134 can be used to hybridize to the product amplified with the primer pair of Groups 73, 87, 95, 103, and 104 depicted in Table 2. The forward primer of SEQ ID NO:97 can be used in the primer pair of Groups 73, 87, 95, 103, and 104 depicted in Table 2 as a second forward primer in the hybridizations in which the first forward primer of SEQ ID NO:97 does not hybridize efficiently. In Table 2, SEQ ID NO:135 is an internal control, not a BKV probe, for binding to an internal control target sequence comprising SEQ ID NO:335 (GCATCGTCCT TTGTAACGAT CAAGGCTGG) for monitoring (1) the efficiency of extraction of nucleic acids from a sample; and (2) the amplification step of PCR.

TABLE 3
Optimized Primers and Probes for Detecting BKV

| | | | |
|---|---|---|---|
| No. | Forward Primer | Probe Group | Reverse Primer |

| | | | |
|---|---|---|---|
| 1 | AGACTGCTAGGTAAGTAAT | AAGTGCTAGAGCAGCACCCAT | AGGGATGCAATTTGAAC |
| | SEQ ID NO: 147 | SEQ ID NO: 186 | SEQ ID NO: 234 |
| 2 | AAGACTCCTAGGTAAGTAAT | CCAAAAGTGCTAGAGCAGCACCCAT | TCAGATACACTGGCAACTA |
| | SEQ ID NO: 146 | SEQ ID NO: 188 | SEQ ID NO: 217 |
| 3 | GACTCCTAGGTAAGTAAT | AAGTGCTAGAGCAGCACCCAT | CCAGCAGCAATTTCA |
| | SEQ ID NO: 148 | SEQ ID NO: 186 | SEQ ID NO: 225 |
| 4 | CATATGCTGTAATTGCT | TAACAGTTTGAATTAAAGCAGCAAACC | GCCTACAGTGGAAACT |
| | SEQ ID NO: 172 | SEQ ID NO: 210 | SEQ ID NO: 250 |
| 5 | TAAAAGACTCCTAGGTAAGTA | AAAGTGCTAGAGCAGCACCCA | CAGATACACTGGCAACTA |
| | SEQ ID NO: 138 | SEQ ID NO: 187 | SEQ ID NO: 216 |
| 6 | TAAAAGACTCCTAGGTAAGTAA | AAAGTGCTAGAGCAGCACCCA | AGCAGCAGCCTCAGA |
| | SEQ ID NO: 139 | SEQ ID NO: 187 | SEQ ID NO: 221 |
| 7 | GTGCTGCTCTAGCACTTT | AGCCTCAGATACACTGGCAACTAGG | CCCCAGCAGCAATTTCA |
| | SEQ ID NO: 150 | SEQ ID NO: 194 | SEQ ID NO: 227 |
| 8 | AAGACTCCTAGGTAAGTAA | AAGTGCTAGAGCAGCACCCAT | GGGATGCAATTTGAACT |
| | SEQ ID NO: 145 | SEQ ID NO: 186 | SEQ ID NO: 233 |
| 9 | AAAGACTCCTAGGTAAGTAA | AAGTGCTAGAGCAGCACCCAT | CCCAGCAGCAATTTCA |
| | SEQ ID NO: 142 | SEQ ID NO: 186 | SEQ ID NO: 226 |
| 10 | AACATATGCTGTAATTGCT | TAACAGTTTGAATTAAAGCAGCAAACCC | CAGTGGAAACTTTGTGAT |
| | SEQ ID NO: 170 | SEQ ID NO: 211 | SEQ ID NO: 248 |
| 11 | CTGCTCTAGCACTTTTGG | CAGCCTCAGATACACTGGCAACTAGGTC | CTCCCCAGCAGCAATTT |
| | SEQ ID NO: 151 | SEQ ID NO: 196 | SEQ ID NO: 230 |
| 12 | AAATTGCATCCCTTGCT | ACCAGCAATTACAGCATATGTTTGAG | GAATTAAAGCAGCAAACC |
| | SEQ ID NO: 158 | SEQ ID NO: 201 | SEQ ID NO: 241 |
| 13 | GTGCTGCTCTAGCACTTT | CAGATACACTGGCAACTAGGTCCCCC | CAGTAGCAAGGGATGCAA |
| | SEQ ID NO: 150 | SEQ ID NO: 193 | SEQ ID NO: 238 |
| 14 | AGACTCCTAGGTAAGTAAT | CAACTAGGTCCCCCAAAAGTGCTAGAG | GCAGCCTCAGATACACT |
| | SEQ ID NO: 147 | SEQ ID NO: 189 | SEQ ID NO: 218 |
| 15 | AAGACTCCTAGGTAAGTAAT | CAGCCTCAGATACACTGGCAACTAGGTC | TCCCCAGCAGCAATTT |
| | SEQ ID NO: 146 | SEQ ID NO: 196 | SEQ ID NO: 228 |
| 16 | TAAAAGACTCCTAGGTAA | AAAGTGCTAGAGCAGCACCCA | GCAAGGGATGCAATTT |
| | SEQ ID NO: 136 | SEQ ID NO: 187 | SEQ ID NO: 237 |
| 17 | CATATGCTGTAATTGCT | CAGTTTGAATTAAAGCAGCAAACC | CTACAGTGGAAACTTTG |
| | SEQ ID NO: 172 | SEQ ID NO: 207 | SEQ ID NO: 249 |
| 18 | TAAAAGACTCCTAGGTAAGTA | CCAAAAGTGCTAGAGCAGCACCCAT | CAGCAGCAGCCTCAGA |
| | SEQ ID NO: 138 | SEQ ID NO: 188 | SEQ ID NO: 223 |
| 19 | AAAGACTCCTAGGTAAGTAAT | CAACTAGGTCCCCCAAAAGTGCTAGAG | AGCAGCCTCAGATACAC |
| | SEQ ID NO: 143 | SEQ ID NO: 189 | SEQ ID NO: 219 |
| 20 | CAAATTGCATCCCTTGCTA | AGCACCAGCAATTACAGCATATGTTTGAG | GTTTGAATTAAAGCAGCAAA |
| | SEQ ID NO: 157 | SEQ ID NO: 202 | SEQ ID NO: 243 |
| 21 | TAAAAGACTCCTAGGTAAGTAA | TACACTGGCAACTAGGTCCCCCAAAAG | CAGCAGCAGCCTCAGA |
| | SEQ ID NO: 139 | SEQ ID NO: 191 | SEQ ID NO: 223 |
| 22 | GTGCTGCTCTAGCACTTT | CAGCCTCAGATACACTGGCAACTAGGTC | AGGGATGCAATTTGAACT |
| | SEQ ID NO: 150 | SEQ ID NO: 196 | SEQ ID NO: 235 |
| 23 | GTGCTGCTCTAGCACTTT | TACACTGGCAACTAGGTCCCCCA | CCTCCCCAGCAGCAAT |
| | SEQ ID NO: 150 | SEQ ID NO: 190 | SEQ ID NO: 232 |
| 24 | AGACTCCTAGGTAAGTAAT | AAGTGCTAGAGCAGCACCCAT | GGGATGCAATTTGAACT |
| | SEQ ID NO: 147 | SEQ ID NO: 186 | SEQ ID NO: 233 |
| 25 | AAGACTCCTAGGTAAGTAAT | AAAGTGCTAGAGCAGCACCCA | GCAAGGGATGCAATTT |
| | SEQ ID NO: 146 | SEQ ID NO: 187 | SEQ ID NO: 237 |
| 26 | AAAGACTCCTAGGTAAGTAA | TACACTGGCAACTAGGTCCCCCAAAAG | CAGCAGCAGCCTCAGA |
| | SEQ ID NO: 142 | SEQ ID NO: 191 | SEQ ID NO: 223 |
| 27 | GACTCCTAGGTAAGTAAT | AAGTGCTAGAGCAGCACCCAT | AGATACACTGGCAACT |
| | SEQ ID NO: 148 | SEQ ID NO: 186 | SEQ ID NO: 215 |
| 28 | TAAAAGACTCCTAGGTAAGTA | AAGTGCTAGAGCAGCACCCAT | CAGATACACTGGCAACTA |
| | SEQ ID NO: 138 | SEQ ID NO: 186 | SEQ ID NO: 216 |
| 29 | TAAAAGACTCCTAGGTAAGTAA | CAGCCTCAGATACACTGGCAACTAGGTC | CTCCCCAGCAGCAATTT |
| | SEQ ID NO: 139 | SEQ ID NO: 196 | SEQ ID NO: 230 |
| 30 | ATTGCATCCCTTGCTACT | AGCACCAGCAATTACAGCATATGTTTGAG | TGAATTAAAGCAGCAAACC |
| | SEQ ID NO: 162 | SEQ ID NO: 202 | SEQ ID NO: 242 |
| 31 | GTGCTGCTCTAGCACTTT | AGATACACTGGCAACTAGGTCCCCC | CCCCAGCAGCAATTTCA |
| | SEQ ID NO: 150 | SEQ ID NO: 192 | SEQ ID NO: 227 |
| 32 | TAAAAGACTCCTAGTAAG | AAAGTGCTAGAGCAGCACCCA | AGATACACTGGCAACT |
| | SEQ ID NO: 137 | SEQ ID NO: 187 | SEQ ID NO: 215 |
| 33 | AAAAGACTCCTAGGTAAGTA | CCAAAAGTGCTAGAGCAGCACCCAT | AGCAGCAGCCTCAGAT |
| | SEQ ID NO: 140 | SEQ ID NO: 188 | SEQ ID NO: 222 |
| 34 | GCATCCCTTGCTACTG | ACCAGCAATTACAGCATATGTTTGAG | GAATTAAAGCAGCAAACC |
| | SEQ ID NO: 166 | SEQ ID NO: 201 | SEQ ID NO: 241 |
| 35 | TTGCCAGTGTATCTGA | AGCAGCAGCCTCCCCAGCAG | AGGGATGCAATTTGAAC |
| | SEQ ID NO: 156 | SEQ ID NO: 200 | SEQ ID NO: 234 |
| 36 | AAAGACTCCTAGGTAAGTAAT | AAAGTGCTAGAGCAGCACCCA | AGCAGCAGCCTCAGA |
| | SEQ ID NO: 143 | SEQ ID NO: 187 | SEQ ID NO: 221 |
| 37 | TGCATCCCTTGCTACTG | AGCACCAGCAATTACAGCATATGTTTGAG | TGAATTAAAGCAGCAAACC |
| | SEQ ID NO: 165 | SEQ ID NO: 202 | SEQ ID NO: 242 |
| 38 | GTGCTGCTCTAGCACTT | CAGCCTCAGATACACTGGCAACTAGGTC | CTCCCCAGCAGCAATTT |
| | SEQ ID NO: 149 | SEQ ID NO: 196 | SEQ ID NO: 230 |
| 39 | AAAAGACTCCTAGGTAAGTAAT | TACACTGGCAACTAGGTCCCCCAAAAG | CAGCAGCAGCCTCAGA |
| | SEQ ID NO: 141 | SEQ ID NO: 191 | SEQ ID NO: 223 |
| 40 | AGACTCCTAGGTAAGTAAT | AAAGTGCTAGAGCAGCACCCA | AAGGGATGCAATTTGA |
| | SEQ ID NO: 147 | SEQ ID NO: 187 | SEQ ID NO: 236 |
| 41 | AAGACTCCTAGGTAAGTAAT | AAGTGCTAGAGCAGCACCCAT | CTCCCCAGCAGCAAT |
| | SEQ ID NO: 146 | SEQ ID NO: 186 | SEQ ID NO: 229 |
| 42 | TCAAACATATGCTGTAATTG | TAACAGTTTGAATTAAAGCAGCAAAC | AGGCCTACAGTGGAAA |
| | SEQ ID NO: 168 | SEQ ID NO: 209 | SEQ ID NO: 251 |
| 43 | ACATATGCTGTAATTGCT | TAACAGTTTGAATTAAAGCAGCAAACCC | CAGTGGAAACTTTGTGAT |
| | SEQ ID NO: 171 | SEQ ID NO: 211 | SEQ ID NO: 248 |
| 44 | TAAAAGACTCCTAGGTAAGTA | CAACTAGGTCCCCCAAAAGTGCTAGAG | CAGCAGCCTCAGATACA |
| | SEQ ID NO: 138 | SEQ ID NO: 189 | SEQ ID NO: 220 |
| 45 | AATTGCATCCCTTGCTA | ACCAGCAATTACAGCATATGTTTGAG | GAATTAAAGCAGCAAACC |
| | SEQ ID NO: 160 | SEQ ID NO: 201 | SEQ ID NO: 241 |
| 46 | TGCATCCCTTGCTACT | TAACAGTTTGAATTAAAGCAGCAAACCC | AGGCCTACAGTGGAAAC |
| | SEQ ID NO: 164 | SEQ ID NO: 211 | SEQ ID NO: 252 |
| 47 | TAAAAGACTCCTAGGTAAGTAA | AAAGTGCTAGAGCAGCACCCA | CCTCCCCAGCAGCAA |
| | SEQ ID NO: 139 | SEQ ID NO: 187 | SEQ ID NO: 231 |
| 48 | GTGCTGCTCTAGCACTTT | TACACTGGCAACTAGGTCCCCCA | AGGGATGCAATTTGAACT |
| | SEQ ID NO: 150 | SEQ ID NO: 190 | SEQ ID NO: 235 |
| 49 | GTGCTGCTCTAGCACTTT | TACACTGGCAACTAGGTCCCCCA | CAGCAGCAGCCTCAGA |
| | SEQ ID NO: 150 | SEQ ID NO: 190 | SEQ ID NO: 223 |
| 50 | AAGACTCCTAGGTAAGTA | AAGTGCTAGAGCAGCACCCAT | GGGATGCAATTTGAACT |
| | SEQ ID NO: 144 | SEQ ID NO: 186 | SEQ ID NO: 233 |
| 51 | AAAGACTCCTAGGTAAGTAA | CAACTAGGTCCCCCAAAAGTGCTAGAG | CAGCAGCAGCCTCAGA |
| | SEQ ID NO: 142 | SEQ ID NO: 189 | SEQ ID NO: 223 |
| 52 | AACATATGCTGTAATTGCT | TAACAGTTTGAATTAAAGCAGCAAACCC | AGGCCTACAGTGGAAAC |
| | SEQ ID NO: 170 | SEQ ID NO: 211 | SEQ ID NO: 252 |
| 53 | TTGCATCCCTTGCTACTG | AGCACCAGCAATTACAGCATATGTTTGAG | TGAATTAAAGCAGCAAACC |
| | SEQ ID NO: 163 | SEQ ID NO: 202 | SEQ ID NO: 242 |
| 54 | GTGCTGCTCTAGCACTTT | AGCCTCAGATACACTGGCAACTAGGTC | TCCCCAGCAGCAATTT |
| | SEQ ID NO: 150 | SEQ ID NO: 195 | SEQ ID NO: 228 |
| 55 | AGACTCCTAGGTAAGTAAT | AAGTGCTAGAGCAGCACCCAT | CCCAGCAGCAATTTCA |
| | SEQ ID NO: 147 | SEQ ID NO: 186 | SEQ ID NO: 226 |
| 56 | AAAAGACTCCTAGGTAAGTA | CCAAAAGTGCTAGAGCAGCACCCAT | TCAGATACACTGGCAACTA |
| | SEQ ID NO: 140 | SEQ ID NO: 188 | SEQ ID NO: 217 |
| 57 | CATATGCTGTAATTGCT | ACAGTTTGAATTAAAGCAGCAAAC | AGTGGAAACTTTGTGA |
| | SEQ ID NO: 172 | SEQ ID NO: 208 | SEQ ID NO: 247 |
| 58 | AAAGACTCCTAGGTAAGTAAT | CAGCCTCAGATACACTGGCAACTAGGTC | CTCCCCAGCAGCAATTT |
| | SEQ ID NO: 143 | SEQ ID NO: 196 | SEQ ID NO: 230 |
| 59 | TAAAAGACTCCTAGGTAAGTAA | CAACTAGGTCCCCCAAAAGTGCTAGAG | CAGCAGCAGCCTCAGAT |
| | SEQ ID NO: 139 | SEQ ID NO: 189 | SEQ ID NO: 224 |
| 60 | GTGCTGCTCTAGCACTTT | TCCCCAGCAGCAATTTCAGCCA | AGGGATGCAATTTGAACT |
| | SEQ ID NO: 150 | SEQ ID NO: 198 | SEQ ID NO: 235 |
| 61 | GTGCTGCTCTAGCACTTT | AGCCTCAGATACACTGGCAACTAGGTC | CCTCCCCAGCAGCAAT |
| | SEQ ID NO: 150 | SEQ ID NO: 195 | SEQ ID NO: 232 |
| 62 | GTTTGCTGCTTTAATTC | TCACAAAGTTTCCACTGTAGGC | TGCCTGATTGCTGAT |
| | SEQ ID NO: 184 | SEQ ID NO: 213 | SEQ ID NO: 253 |
| 63 | TCAAACATATGCTGTAATTG | CCTACAGTGGAAACTTTGTGATCC | ATGCCTGATTGCTGAT |
| | SEQ ID NO: 168 | SEQ ID NO: 214 | SEQ ID NO: 255 |
| 64 | GCTATTGCTGGGTTTG | CCTACAGTGGAAACTTTGTGATCC | ATGCCTGATTGCTGAT |
| | SEQ ID NO: 175 | SEQ ID NO: 214 | SEQ ID NO: 255 |
| 65 | GGCTATTGCTGGGTTT | CCTACAGTGGAAACTTTGTGATCC | TGCCTGATTGCTGAT |
| | SEQ ID NO: 173 | SEQ ID NO: 214 | SEQ ID NO: 253 |
| 66 | TGCATCCCTTGCTACT | CCTACAGTGGAAACTTTGTGATCC | ATGCCTGATTGCTGAT |
| | SEQ ID NO: 164 | SEQ ID NO: 214 | SEQ ID NO: 255 |
| 67 | TATTGCTGGGTTTGC | CCTACAGTGGAAACTTTGTGATCC | TGCCTGATTGCTGAT |
| | SEQ ID NO: 177 | SEQ ID NO: 214 | SEQ ID NO: 253 |
| 68 | CAAACATATGCTGTAATTG | TTGAATTAAAGCAGCAAACCCA | TGCCTGATTGCTGAT |
| | SEQ ID NO: 169 | SEQ ID NO: 204 | SEQ ID NO: 253 |
| 69 | GGTTTGCTGCTTTAATTC | CCTACAGTGGAAACTTTGTGATCC | ATGCCTGATTGCTGAT |
| | SEQ ID NO: 183 | SEQ ID NO: 214 | SEQ ID NO: 255 |
| 70 | CATATGCTGTAATTGCT | AGTTTGAATTAAAGCAGCAAACCC | TGATCCCAATCACTAAA |
| | SEQ ID NO: 172 | SEQ ID NO: 206 | SEQ ID NO: 245 |
| 71 | GCATCCTTGCTACTG | CCTACAGTGGAAACTTTGTGATCC | TGCCTGATTGCTGATA |
| | SEQ ID NO: 166 | SEQ ID NO: 214 | SEQ ID NO: 254 |
| 72 | ATTGCTGGGTTTGCT | CCTACAGTGGAAACTTTGTGATCC | TGCCTGATTGCTGATA |
| | SEQ ID NO: 178 | SEQ ID NO: 214 | SEQ ID NO: 254 |
| 73 | TGCATCCCTTGCTACTG | CCTACAGTGGAAACTTTGTGATCC | ATGCCTGATTGCTGAT |
| | SEQ ID NO: 165 | SEQ ID NO: 214 | SEQ ID NO: 255 |
| 74 | GCTATTGCTGGGTTT | CCTACAGTGGAAACTTTGTGATCC | TGCCTGATTGCTGAT |
| | SEQ ID NO: 174 | SEQ ID NO: 214 | SEQ ID NO: 253 |
| 75 | TATTGCTGGGTTTGC | ATCACAAAGTTTCCACTGTAGGC | TGCCTGATTGCTGAT |
| | SEQ ID NO: 177 | SEQ ID NO: 212 | SEQ ID NO: 253 |
| 76 | ACATATGCTGTAATTGCT | CCTACAGTGGAAACTTTGTGATCC | TGCCTGATGCTGATA |
| | SEQ ID NO: 171 | SEQ ID NO: 214 | SEQ ID NO: 254 |
| 77 | AATTGCATCCCTTGCTA | CCTACAGTGGAAACTTTGTGATCC | ATGCCTGATTGCTGAT |
| | SEQ ID NO: 160 | SEQ ID NO: 214 | SEQ ID NO: 255 |
| 78 | CATATGCTGTAATTGCT | TAACAGTTTGAATTAAAGCAGCAAAC | GTGATCCCAATCACTAA |
| | SEQ ID NO: 172 | SEQ ID NO: 209 | SEQ ID NO: 246 |
| 79 | GCTATTGCTGGGTTTG | CCTACAGTGGAAACTTTGTGATCC | TGCCTGATTGCTGATA |
| | SEQ ID NO: 175 | SEQ ID NO: 214 | SEQ ID NO: 254 |
| 80 | ATTGCATCCCTTGCT | CCTACAGTGGAAACTTTGTGATCC | TGCCTGATTGCTGAT |
| | SEQ ID NO: 161 | SEQ ID NO: 214 | SEQ ID NO: 253 |
| 81 | AATTGCATCCCTTGCT | CCTACAGTGGAAACTTTGTGATCC | ATGCCTGATTGCTGAT |
| | SEQ ID NO: 159 | SEQ ID NO: 214 | SEQ ID NO: 255 |
| 82 | CTCAAACATATGCTGTAAT | TGAATTAAAGCAGCAAACCCA | TGCCTGATTGCTGAT |
| | SEQ ID NO: 167 | SEQ ID NO: 203 | SEQ ID NO: 253 |
| 83 | GGTTTGCTGCTTTAATTC | CCTACAGTGGAAACTTTGTGATCC | TGCCTGATTGCTGATA |
| | SEQ ID NO: 183 | SEQ ID NO: 214 | SEQ ID NO: 254 |
| 84 | CTGGGTTTGCTGCTTTA | CCTACAGTGGAAACTTTGTGATCC | ATGCCTGATTGCTGAT |
| | SEQ ID NO: 181 | SEQ ID NO: 214 | SEQ ID NO: 255 |
| 85 | GTGCTGCTCTAGCACTTT | CTCCCCAGCAGCAATTTCAGCCA | AGCAGCTATAGCCTCTGA |
| | SEQ ID NO: 150 | SEQ ID NO: 199 | SEQ ID NO: 240 |
| 86 | CTGGGTTTGCTGCTT | ATCACAAAGTTTCCACTGTAGGC | TGCCTGATTGCTGAT |
| | SEQ ID NO: 179 | SEQ ID NO: 212 | SEQ ID NO: 253 |
| 87 | CATATGCTGTAATTGCT | CCTACAGTGGAAACTTTGTGATCC | TGCCTGATTGCTGATA |
| | SEQ ID NO: 172 | SEQ ID NO: 214 | SEQ ID NO: 254 |
| 88 | CTATTGCTGGGTTTGC | CCTACAGTGGAAACTTTGTGATCC | TGCCTGATTGCTGAT |
| | SEQ ID NO: 176 | SEQ ID NO: 214 | SEQ ID NO: 253 |
| 89 | CAAACATATGCTGTAATTG | CCTACAGTGGAAACTTTGTGATCC | TGCCTGATTGCTGAT |
| | SEQ ID NO: 169 | SEQ ID NO: 214 | SEQ ID NO: 253 |
| 90 | GGTTTGCTGCTTTAATTC | CCTACAGTGGAAACTTTGTGATCC | TGCCTGATTGCTGAT |
| | SEQ ID NO: 183 | SEQ ID NO: 214 | SEQ ID NO: 253 |
| 91 | CATATGCTGTAATTGCT | AGTTTGAATTAAAGCAGCAAACC | GTGATCCCAATCACTAA |
| | SEQ ID NO: 172 | SEQ ID NO: 205 | SEQ ID NO: 246 |
| 92 | GCATCCCTTGCTACTG | CCTACAGTGGAAACTTTGTGATCC | ATGCCTGATTGCTGAT |
| | SEQ ID NO: 166 | SEQ ID NO: 214 | SEQ ID NO: 255 |
| 93 | CCTAGTTGCCAGTGTA | AGCAGCAATTTCAGCCACTG | CAGCTATAGCCTCTGA |
| | SEQ ID NO: 154 | SEQ ID NO: 197 | SEQ ID NO: 239 |
| 94 | ATTGCTGGGTTTGCT | CCTACAGTGGAAACTTTGTGATCC | TGCCTGATTGCTGAT |
| | SEQ ID NO: 178 | SEQ ID NO: 214 | SEQ ID NO: 253 |
| 95 | TGGGGGACCTAGTTG | AGCAGCAATTTCAGCCACTG | CAGCTATAGCCTCTGA |
| | SEQ ID NO: 153 | SEQ ID NO: 197 | SEQ ID NO: 239 |
| 96 | CTTTTGGGGGACCTA | AGCAGCAATTTCAGCCACTG | CAGCTATAGCCTCTGA |
| | SEQ ID NO: 152 | SEQ ID NO: 197 | SEQ ID NO: 239 |
| 97 | GCTATTGCTGGGTTT | ATCACAAAGTTTCCACTGTAGGC | TGCCTGATTGCTGAT |
| | SEQ ID NO: 174 | SEQ ID NO: 212 | SEQ ID NO: 253 |
| 98 | CTGCTTTAATTCAAACTGT | CCTACAGTGGAAACTTTGTGATCC | TGCCTGATTGCTGATA |
| | SEQ ID NO: 185 | SEQ ID NO: 214 | SEQ ID NO: 254 |
| 99 | ACATATGCTGTAATTGCT | CCTACAGTGGAAACTTTGTGATCC | ATGCCTGATTGCTGAT |
| | SEQ ID NO: 171 | SEQ ID NO: 214 | SEQ ID NO: 255 |
| 100 | AGTTGCCAGTGTATCT | AGCAGCAATTTCAGCCACTG | CAGCTATAGCCTCTGA |
| | SEQ ID NO: 155 | SEQ ID NO: 197 | SEQ ID NO: 239 |
| 101 | TGGGTTTGCTGCTTTA | CCTACAGTGGAAACTTTGTGATCC | ATGCCTGATTGCTGAT |
| | SEQ ID NO: 182 | SEQ ID NO: 214 | SEQ ID NO: 255 |
| 102 | CTGGGTTTGCTGCTTT | CCTACAGTGGAAAGTTTGTGATCC | TGCCTGATTGCTGATA |
| | SEQ ID NO: 180 | SEQ ID NO: 214 | SEQ ID NO: 254 |
| 103 | CATATGCTGTAATTGCT | TAACAGTTTGAATTAAAGCAGCAAAC | ACCTATACCCTACTTGAG |
| | SEQ ID NO: 172 | SEQ ID NO: 209 | SEQ ID NO: 244 |
| 104 | GGTTTGCTGCTTTAATTC | AAGATTCAGCAGCTACCGCAGCCC | TGCCTGATTGCTGAT |
| | SEQ ID NO: 183 | SEQ ID NO: 334 | SEQ ID NO: 253 |

A PCR primer pair for amplifying BK virus DNA can comprise any one of the following pairs of primer sequences: (1) SEQ ID NOS: 147 and 234; (2) SEQ ID NOS: 146 and 217; (3) SEQ ID NOS: 148 and 225; (4) SEQ ID NOS: 172 and 250; (5) SEQ ID NOS: 138 and 216; (6) SEQ ID NOS: 139 and 221; (7) SEQ ID NOS: 150 and 227; (8) SEQ ID NOS: 145 and 233; (9) SEQ ID NOS: 142 and 226; (10) SEQ ID NOS: 170 and 248; (11) SEQ ID NOS: 151 and 230; (12) SEQ ID NOS: 158 and 241; (13) SEQ ID NOS: 150 and 238; (14) SEQ ID NOS: 147 and 218; (15) SEQ ID NOS: 146 and 228; (16) SEQ ID NOS: 136 and 237; (17) SEQ ID NOS: 172 and 249; (18) SEQ ID NOS: 138 and 223; (19) SEQ ID NOS: 143 and 219; (20) SEQ ID NOS: 157 and 243; (21) SEQ ID NOS: 139 and 223; (22) SEQ ID NOS: 150 and 235; (23) SEQ ID NOS: 150 and 232; (24) SEQ ID NOS: 147 and 233; (25) SEQ ID NOS: 146 and 237; (26) SEQ ID NOS: 142 and 223; (27) SEQ ID NOS: 148 and 215; (28) SEQ ID NOS: 139 and 230; (29) SEQ ID NOS: 162 and 242; (30) SEQ ID NOS: 137 and 215; (31) SEQ ID NOS: 140 and 222; (32) SEQ ID NOS: 166 and 241; (33) SEQ ID NOS: 156 and 234; (34) SEQ ID NOS: 143 and 221; (35) SEQ ID NOS: 165 and 242; (36) SEQ ID NOS: 149 and 230; (37) SEQ ID NOS: 141 and 223; (38) SEQ ID NOS: 147 and 236; (39) SEQ ID NOS: 146 and 229; (40) SEQ ID NOS: 168 and 251, (41) SEQ ID NOS: 171 and 248; (42) SEQ ID NOS: 138 and 220; (43) SEQ ID NOS: 160 and 241; (44) SEQ ID NOS: 164 and 252; (45) SEQ ID NOS: 139 and 231; (46) SEQ ID NOS: 150 and 223; (47) SEQ ID NOS: 144 and 233; (48) SEQ ID NOS: 170 and 252; (49) SEQ ID NOS: 163 and 242; (50) SEQ ID NOS: 150 and 228; (51) SEQ ID NOS: 147 and 226; (52) SEQ ID NOS: 140 and 217; (53) SEQ ID NOS: 172 and 247; (54) SEQ ID NOS: 143 and 230; (55) SEQ ID NOS: 139 and 224; (56) SEQ ID NOS: 150 and 235; (57) SEQ ID NOS: 184 and 253; (58) SEQ ID NOS: 168 and 255; (59) SEQ ID NOS: 175 and 255; (60) SEQ ID NOS: 173 and 253; (61) SEQ ID NOS: 164 and 255; (62) SEQ ID NOS: 177 and 253; (63) SEQ ID NOS: 169 and 253; (64) SEQ ID NOS: 183 and 255; (65) SEQ ID NOS: 172 and 245; (66) SEQ ID NOS: 166 and 254; (67) SEQ ID NOS: 178 and 254; (68) SEQ ID NOS: 165 and 255; (69) SEQ ID NOS: 174 and 253; (70) SEQ ID NOS: 171 and 254; (71) SEQ ID NOS: 160 and 255; (72) SEQ ID NOS: 172 and 246; (73) SEQ ID NOS: 175 and 254; (74) SEQ ID NOS: 161 and 253; (75) SEQ ID NOS: 159 and 255; (76) SEQ ID NOS: 167 and 253; (77) SEQ ID NOS: 183 and 254; (78) SEQ ID NOS: 181 and 255; (79) SEQ ID NOS: 150 and 240; (80) SEQ ID NOS: 179 and 253; (81) SEQ ID NOS: 172 and 254; (82) SEQ ID NOS: 176 and 253; (83) SEQ ID NOS: 183 and 253; (84) SEQ ID NOS: 166 and 255; (85) SEQ ID NOS: 154 and 239; (86) SEQ ID NOS: 178 and 253; (87) SEQ ID NOS: 153 and 239; (88) SEQ ID NOS: 152 and 239; (89) SEQ ID NOS: 185 and 254; (90) SEQ ID NOS: 171 and 255; (91) SEQ ID NOS: 155 and 239; (92) SEQ ID NOS: 182 and 255; (93) SEQ ID NOS:180 and 254; (94) SEQ ID NOS: 172 and 244; and (95) SEQ ID NOS: 183 and 253.

The preceding numbering of the ninety-five pairs of primers does not correspond exactly to the “Group” numbering scheme in Table 3 because certain groups use the same primer pair, but different internal probes. For example, Groups 5 and 28 of Table 3 each employ the forward primer of SEQ ID NO:138 and the reverse primer of SEQ ID NO:216, but two different internal probes in each instance, e.g., SEQ ID NOS: 187 and 186. Accordingly, primer pair “(5)” of the preceding passage implies any one of Groups 5 and 28 of Table 3. Likewise, Groups 26 and 51 of Table 3 each employ the primers of SEQ ID NOS: 142 and 223, but one Group uses the probe of SEQ ID NO:191 whilst the other Group uses the probe of SEQ ID NO:189. Groups 26 and 51 are depicted in primer pair “(26)” in the preceding passage.

The probe of SEQ ID NO:186 can be used to hybridize to the product amplified with the primer pair of Groups 1, 3, 8, 9, 24, 27, 28, 41, 50 and 55 depicted in Table 3. The probe of SEQ ID NO:187 can be used to hybridize to the product amplified with the primer pair of Groups 5, 6, 16, 25, 32, 36, 40, and 47 depicted in Table 3. The probe of SEQ ID NO:188 can be used to hybridize to the product amplified with the primer pair of Groups 2, 18, 33, and 56 depicted in Table 3 The probe of SEQ ID NO:189 can be used to hybridize to the product amplified with the primer pair of Groups 14, 19, 44, 51, and 59 depicted in Table 3. The probe of SEQ ID NO:190 can be used to hybridize to the product amplified with the primer pair of Groups 23, 48, and 49 depicted in Table 3. The probe of SEQ ID NO:191 can be used to hybridize to the product amplified with the primer pair of Groups 21, 26, and 39 depicted in Table 3. The probe of SEQ ID NO:195 can be used to hybridize to the product amplified with the primer pair of Groups 54 and 61 depicted in Table 3. The probe of SEQ ID NO:197 can be used to hybridize to the product amplified with the primer pair of Groups 93, 95, 96, and 100 depicted in Table 3. The probe of SEQ ID NO:198 can be used to hybridize to the product amplified with the primer pair of Group 60 depicted in Table 3. The probe of SEQ ID NO:199 can be used to hybridize to the product amplified with the primer pair of Group 85 depicted in Table 3. The probe of SEQ ID NO:200 can be used to hybridize to the product amplified with the primer pair of Group 35 depicted in Table 3. The probe of SEQ ID NO:201 can be used to hybridize to the product amplified with the primer pair of Groups 12, 34, and 45 depicted in Table 3. The probe of SEQ ID NO:202 can be used to hybridize to the product amplified with the primer pair of Groups 20, 30, 37, and 53 depicted in Table 3. The probe of SEQ ID NO:203 can be used to hybridize to the product amplified with the primer pair of Group 82 depicted in Table 3. The probe of SEQ ID NO:204 can be used to hybridize to the product amplified with the primer pair of Group 68 depicted in Table 3. The probe of SEQ ID NO:205 can be used to hybridize to the product amplified with the primer pair of Group 91 depicted in Table 3. The probe of SEQ ID NO:206 can be used to hybridize to the product amplified with the primer pair of Group 70 depicted in Table 3 The probe of SEQ ID NO:207 can be used to hybridize to the product amplified with the primer pair of Group 17 depicted in Table 3. The probe of SEQ ID NO:208 can be used to hybridize to the product amplified with the primer pair of Group 57 depicted in Table 3. The probe of SEQ ID NO:209 can be used to hybridize to the product amplified with the primer pair of Groups 42, 78, and 103 depicted in Table 3. The probe of SEQ ID NO:210 can be used to hybridize to the product amplified with the primer pair of Group 4 depicted in Table 3. The probe of SEQ ID NO:211 can be used to hybridize to the product amplified with the primer pair of Groups 10, 43, 46, and 52 depicted in Table 3. The probe of SEQ ID NO:212 can be used to hybridize to the product amplified with the primer pair of Groups 75, 86, and 97 depicted in Table 3. The probe of SEQ ID NO:213 can be used to hybridize to the product amplified with the primer pair of Group 62 depicted in Table 3. The probe of SEQ ID NO:214 can be used to hybridize to the product amplified with the primer pair of Group 63-67, 69, 71-74, 76, 77, 79-81, 83, 84, 87-90, 92, 94, 98, 99, 101, and 102 depicted in Table 3. In Table 3 SEQ ID NO:334 is an internal control probe, not a BKV probe, for binding to an internal control target sequence comprising SEQ ID NO:336 (GGGCTGCGGTAGCTGCTGAATCTT) for monitoring (1) the efficiency of extraction of nucleic acids from a sample; and (2) the amplification step of PCR.

TABLE 4
Optimized Primers and Probes for Detecting BKV

| | | | |
|---|---|---|---|
| No. | Forward Primer | Probe Group | Reverse Primer |

| | | | |
|---|---|---|---|
| 1 | CAATGCAGTAGCAATCTA | TGATTTTCCTCTTTGCCCAGATAC | TAGCTCAGAGGTTTGTG |
| | SEQ ID NO: 256 | SEQ ID NO: 282 | SEQ ID NO: 292 |
| 2 | AATGCAGTAGCAATCTAT | TGATTTTCCTCTTTGCCCAGATA | TAGCTCAGAGGTTTGTG |
| | SEQ ID NO: 257 | SEQ ID NO: 282 | SEQ ID NO: 292 |
| 3 | AATGCAGTAGCAATCTAT | TGATTTTCCTCTTTGCCCAGATAC | AGCTCAGAGGTTTGTG |
| | SEQ ID NO: 257 | SEQ ID NO: 282 | SEQ ID NO: 293 |
| 4 | AATGCAGTAGCAATCTATC | TGATTTTCCTCTTTGCCCAGATAC | TAGCTCAGAGGTTTGTG |
| | SEQ ID NO: 258 | SEQ ID NO: 282 | SEQ ID NO: 292 |
| 5 | TGCAGTAGCAATCTATCC | TGATTTTCCTCTTTGCCCAGATAC | TAGCTCAGAGGTTTGTG |
| | SEQ ID NO: 259 | SEQ ID NO: 282 | SEQ ID NO: 292 |
| 6 | GCAGTAGCAATCTATCCA | TGATTTTCCTCTTTGCCCAGATAC | TAGCTCAGAGGTTTGTG |
| | SEQ ID NO: 260 | SEQ ID NO: 282 | SEQ ID NO: 292 |
| 7 | CAGTAGCAATCTATCCAAA | TGATTTTCCTCTTTGCCCAGATAC | TAGCTCAGAGGTTTGTG |
| | SEQ ID NO: 261 | SEQ ID NO: 282 | SEQ ID NO: 292 |
| 8 | GCAATCTATCCAAACCAA | TGATTTTCCTCTTTGCCCAGATAC | TAGCTCAGAGGTTTGTG |
| | SEQ ID NO: 262 | SEQ ID NO: 282 | SEQ ID NO: 292 |
| 9 | CAATCTATCCAAACCAA | TGATTTTCCTCTTTGCCCAGATAC | AGCTCAGAGGTTTGTG |
| | SEQ ID NO: 263 | SEQ ID NO: 282 | SEQ ID NO: 293 |
| 10 | CTGGGCAAAGAGGAAA | TCCAGGTTCCAAAATCAGGCT | GGATAAAATGAAGAGAATGA |
| | SEQ ID NO: 264 | SEQ ID NO: 283 | SEQ ID NO: 294 |
| 11 | CTGGGCAAAGAGGAAA | TCCAGGTTCCAAAATCAGGCT | GAGGATAAAATGAAGAGAA |
| | SEQ ID NO: 264 | SEQ ID NO: 283 | SEQ ID NO: 295 |
| 12 | CTGGGCAAAGAGGAAA | TCCAGGTTCCAAAATCAGGCT | ACGAGGATAAAATGAAGAG |
| | SEQ ID NO: 264 | SEQ ID NO: 283 | SEQ ID NO: 296 |
| 13 | CTGGGCAAAGAGGAAA | TCCAGGTTCCAAAATCAGGCT | CGACGAGGATAAAATGAA |
| | SEQ ID NO: 264 | SEQ ID NO: 283 | SEQ ID NO: 297 |
| 14 | CTGGGCAAAGAGGAAA | TCCAGGTTCCAAAATCAGGCT | GGCGACGAGGATAAAAT |
| | SEQ ID NO: 264 | SEQ ID NO: 283 | SEQ ID NO: 298 |
| 15 | CTGGGCAAAGAGGAAA | TCCAGGTTCCAAAATCAGGCT | TTTCACCCTGACAAAGG |
| | SEQ ID NO: 264 | SEQ ID NO: 283 | SEQ ID NO: 299 |
| 16 | CTGGGCAAAGAGGAAA | TCCAGGTTCCAAAATGAGGCT | GGAATTTCACCCTGACA |
| | SEQ ID NO: 264 | SEQ ID NO: 283 | SEQ ID NO: 300 |
| 17 | TGGGCAAAGAGGAAAA | TCCAGGTTCCAAAATCAGGCT | GGATAAAATGAAGAGAATGA |
| | SEQ ID NO: 265 | SEQ ID NO: 283 | SEQ ID NO: 294 |
| 18 | TGGGCAAAGAGGAAAA | TCCAGGTTCCAAAATCAGGCT | GAGGATAAAATGAAGAGAAT |
| | SEQ ID NO: 265 | SEQ ID NO: 283 | SEQ ID NO: 301 |
| 19 | TGGGCAAAGAGGAAAA | TCCAGGTTCCAAAATCAGGCT | CGAGGATAAAATGAAGAGAA |
| | SEQ ID NO: 265 | SEQ ID NO: 283 | SEQ ID NO: 302 |
| 20 | TGGGCAAAGAGGAAAA | TCCAGGTTCCAAAATCAGGCT | CGACGAGGATAAAATGAAG |
| | SEQ ID NO: 265 | SEQ ID NO: 283 | SEQ ID NO: 303 |
| 21 | TGGGCAAAGAGGAAAA | TCCAGGTTCCAAAATCAGGCT | GGGCGACGAGGATAAAA |
| | SEQ ID NO: 265 | SEQ ID NO: 283 | SEQ ID NO: 304 |
| 22 | TGGGCAAAGAGGAAAA | TCCAGGTTCCAAAATCAGGCT | GGGGCGACGAGGATAA |
| | SEQ ID NO: 265 | SEQ ID NO: 283 | SEQ ID NO: 305 |
| 23 | TGGGCAAAGAGGAAAA | TCCAGGTTCCAAAATCAGGCT | TTTCACCCTGACAAAGG |
| | SEQ ID NO: 265 | SEQ ID NO: 283 | SEQ ID NO: 299 |
| 24 | TGGGCAAAGAGGAAAA | TCCAGGTTCCAAAATCAGGCT | AATTTCACCCTGACAA |
| | SEQ ID NO: 265 | SEQ ID NO: 283 | SEQ ID NO: 306 |
| 25 | TGGGCAAAGAGGAAAA | TCCAGGTTCCAAAATCAGGCT | GGAATTTCACCCTGACAA |
| | SEQ ID NO: 265 | SEQ ID NO: 283 | SEQ ID NO: 307 |
| 26 | GGGCAAAGAGGAAAATC | TCCAGGTTCCAAAATCAGGCT | GGATAAAATGAAGAGAATGA |
| | SEQ ID NO: 266 | SEQ ID NO: 283 | SEQ ID NO: 294 |
| 27 | GGGCAAAGAGGAAAATC | TCCAGGTTCCAAAATCAGGCT | GAGGATAAAATGAAGAGAAT |
| | SEQ ID NO: 266 | SEQ ID NO: 283 | SEQ ID NO: 301 |
| 28 | GGGCAAAGAGGAAAATC | TCCAGGTTCCAAAATCAGGCT | ACGAGGATAAAATGAAGAG |
| | SEQ ID NO: 266 | SEQ ID NO: 283 | SEQ ID NO: 296 |
| 29 | GGGCAAAGAGGAAAATC | TCCAGGTTCCAAAATCAGGCT | CGACGAGGATAAAATGAAG |
| | SEQ ID NO: 266 | SEQ ID NO: 283 | SEQ ID NO: 303 |
| 30 | GGGCAAAGAGGAAAATC | TCCAGGTTCCAAAATCAGGCT | GGCGACGAGGATAAAAT |
| | SEQ ID NO: 266 | SEQ ID NO: 283 | SEQ ID NO: 298 |
| 31 | GGGCAAAGAGGAAAATC | TCCAGGTTCCAAAATCAGGCT | GGGGCGACGAGGATA |
| | SEQ ID NO: 266 | SEQ ID NO: 283 | SEQ ID NO: 308 |
| 32 | GGGCAAAGAGGAAAATC | TCCAGGTTCCAAAATCAGGCT | ATTTCACCCTGACAAAG |
| | SEQ ID NO: 266 | SEQ ID NO: 283 | SEQ ID NO: 309 |
| 33 | GGGCAAAGAGGAAAATC | TCCAGGTTCCAAAATCAGGCT | GGAATTTCACCCTGACA |
| | SEQ ID NO: 266 | SEQ ID NO: 283 | SEQ ID NO: 300 |
| 34 | GGCAAAGAGGAAAATCA | TCCAGGTTCCAAAATCAGGCT | GGATAAAATGAAGAGAATGA |
| | SEQ ID NO: 267 | SEQ ID NO: 283 | SEQ ID NO: 294 |
| 35 | GGCAAAGAGGAAAATCA | TCCAGGTTCCAAAATCAGGCT | GAGGATAAAATGAAGAGAAT |
| | SEQ ID NO: 267 | SEQ ID NO: 283 | SEQ ID NO: 301 |
| 36 | GGCAAAGAGGAAAATCA | TCCAGGTTCCAAAATCAGGCT | ACGAGGATAAAATGAAGAG |
| | SEQ ID NO: 267 | SEQ ID NO: 283 | SEQ ID NO: 296 |
| 37 | GGCAAAGAGGAAAATCA | TCCAGGTTCCAAAATGAGGCT | CGACGAGGATAAAATGAAG |
| | SEQ ID NO: 267 | SEQ ID NO: 283 | SEQ ID NO: 303 |
| 38 | GGCAAAGAGGAAAATCA | TCCAGGTTCCAAAATCAGGCT | GGCGACGAGGATAAAAT |
| | SEQ ID NO: 267 | SEQ ID NO: 283 | SEQ ID NO: 298 |
| 39 | GGCAAAGAGGAAAATCA | TCCAGGTTCCAAAATCAGGCT | GGGGCGACGAGGATAA |
| | SEQ ID NO: 267 | SEQ ID NO: 283 | SEQ ID NO: 305 |
| 40 | GGCAAAGAGGAAAATCA | TCCAGGTTCCAAAATCAGGCT | TTTCACCCTGACAAAGG |
| | SEQ ID NO: 267 | SEQ ID NO: 283 | SEQ ID NO: 299 |
| 41 | GGCAAAGAGGAAAATCA | TCCAGGTTCCAAAATCAGGCT | AATTTCACCCTGACAAAG |
| | SEQ ID NO: 267 | SEQ ID NO: 283 | SEQ ID NO: 310 |
| 42 | GGCAAAGAGGAAAATCA | TCCAGGTTCCAAAATCAGGCT | GGAATTTCACCCTGACA |
| | SEQ ID NO: 267 | SEQ ID NO: 283 | SEQ ID NO: 300 |
| 43 | AAATCAGCACAAACCT | TCCAGGTTCCAAAATCAGGCT | GGGGCGACGAGGATA |
| | SEQ ID NO: 268 | SEQ ID NO: 283 | SEQ ID NO: 308 |
| 44 | AAATCAGCACAAACCT | TCCAGGTTCCAAAATCAGGCT | TTTCACCCTGACAAAGG |
| | SEQ ID NO: 268 | SEQ ID NO: 283 | SEQ ID NO: 299 |
| 45 | AAATCAGCACAAACCT | TCCAGGTTCCAAAATCAGGCT | AATTTCACCCTGACAAAG |
| | SEQ ID NO: 268 | SEQ ID NO: 253 | SEQ ID NO: 310 |
| 46 | AAATCAGCACAAACCT | TCCAGGTTCCAAAATCAGGCT | GGAATTTCACCCTGACA |
| | SEQ ID NO: 268 | SEQ ID NO: 283 | SEQ ID NO: 300 |
| 47 | AATCAGCACAAACCT | TCCAGGTTCAAAATCAGGCT | GGATAAAATGAAGAGAATG |
| | SEQ ID NO: 268 | SEQ ID NO: 283 | SEQ ID NO: 311 |
| 48 | AATCAGCACAAACCT | TCCAGGTTCCAAAATCAGGCT | ACGAGGATAAAATGAAG |
| | SEQ ID NO: 269 | SEQ ID NO: 283 | SEQ ID NO: 312 |
| 49 | AATCAGCACAAACCT | TCCAGGTTCCAAAATCAGGCT | CGACGAGGATAAAATGA |
| | SEQ ID NO: 269 | SEQ ID NO: 283 | SEQ ID NO: 313 |
| 50 | AATCAGCACAAACCT | TCCAGGTTCCAAAATCAGGCT | GGGCGACGAGGATAA |
| | SEQ ID NO: 269 | SEQ ID NO: 283 | SEQ ID NO: 314 |
| 51 | AATCAGCAGAAACCT | TCCAGGTTCCAAAATCAGGCT | TCACCCTGACAAAGG |
| | SEQ ID NO: 269 | SEQ ID NO: 283 | SEQ ID NO: 315 |
| 52 | AATCAGCACAAACCT | TCCAGGTTCCAAAATCAGGCT | TTTCACCCTGACAAAG |
| | SEQ ID NO: 269 | SEQ ID NO: 283 | SEQ ID NO: 316 |
| 53 | AATCAGCACAAACCT | TCCAGGTTCCAAAATCAGGCT | AATTTCACCTGACAA |
| | SEQ ID NO: 269 | SEQ ID NO: 283 | SEQ ID NO: 306 |
| 54 | CAGCACAAACCTCTGA | TCCAGGTTCCAAAATCAGGCT | GGATAAAATGAAGAGAATGA |
| | SEQ ID NO: 270 | SEQ ID NO: 283 | SEQ ID NO: 294 |
| 55 | CAGCACAAACCTCTGA | TCCAGGTTCCAAAATCAGGCT | CGACGATAAAATGAAGAGA |
| | SEQ ID NO: 270 | SEQ ID NO: 283 | SEQ ID NO: 317 |
| 56 | CAGCACAAACTCTGA | TCCAGGTTCCAAAATCAGGCT | CGACGAGGATAAAATGAAG |
| | SEQ ID NO: 270 | SEQ ID NO: 283 | SEQ ID NO: 303 |
| 57 | CAGCACAAACCTCTGA | TCCAGGTTCCAAAATCAGGCT | GGCGACGAGGATAAAAT |
| | SEQ ID NO: 270 | SEQ ID NO: 283 | SEQ ID NO: 298 |
| 58 | CAGCACAAACCTCTGA | TCCAGGTTCCAAAATCAGGCT | GGGGCGACGAGGATA |
| | SEQ ID NO: 270 | SEQ ID NO: 283 | SEQ ID NO: 308 |
| 59 | CAGCACAAACCTCTGA | TCCAGGTTCCAAAATCAGGCT | ATTTCACCCTGACAAAG |
| | SEQ ID NO: 270 | SEQ ID NO: 283 | SEQ ID NO: 309 |
| 60 | CAGCACAAACCTCTGA | TCCAGGTTCCAAAATCAGGCT | AATTTCACCCTGACAAAG |
| | SEQ ID NO: 270 | SEQ ID NO: 283 | SEQ ID NO: 310 |
| 61 | CAGCACAAACCTCTGA | TCCAGGTTCCAAAATCAGGCT | GGAATTTCACCCTGACA |
| | SEQ ID NO: 270 | SEQ ID NO: 283 | SEQ ID NO: 300 |
| 62 | CACAAACCTCTGAGCTA | TCCAGGTTCCAAAATCAGGCT | GGATAAAATGAAGAGAATGA |
| | SEQ ID NO: 271 | SEQ ID NO: 283 | SEQ ID NO: 294 |
| 63 | CACAAACCTCTGAGCTA | TCCAGGTTCCAAAATCAGGCT | GAGGATAAAATGAAGAGAAT |
| | SEQ ID NO: 271 | SEQ ID NO: 283 | SEQ ID NO: 301 |
| 64 | CACAAACCTCTGAGCTA | TCCAGGTTCCAAAATCAGGCT | ACGAGGATAAAATGAAGAG |
| | SEQ ID NO: 271 | SEQ ID NO: 283 | SEQ ID NO: 296 |
| 65 | CACAAACCTCTGAGCTA | TCCAGGTTCCAAAATCAGGCT | CGACGAGGATAAAAGAAG |
| | SEQ ID NO: 271 | SEQ ID NO: 283 | SEQ ID NO: 303 |
| 66 | CACAAACCTCTGAGCTA | TCCAGGTTCCAAAATCAGGCT | GGCGACGAGGATAAAAT |
| | SEQ ID NO: 271 | SEQ ID NO: 283 | SEQ ID NO: 298 |
| 67 | CACAAACCTCTGAGCTA | TCCAGGTTCCAAAATCAGGCT | GGGGCGACGAGGATAA |
| | SEQ ID NO: 271 | SEQ ID NO: 283 | SEQ ID NO: 305 |
| 68 | CACAAACCTCTGAGCTA | TCCAGGTTCCAAAATCAGGCT | TTCACCCTGACAAAGG |
| | SEQ ID NO: 271 | SEQ ID NO: 283 | SEQ ID NO: 318 |
| 69 | CACAAACCTCTGAGCTA | TCCAGGTTCCAAAATCAGGCT | ATTTCACCCTGACAAAG |
| | SEQ ID NO: 271 | SEQ ID NO: 283 | SEQ ID NO: 309 |
| 70 | CACAAACCTCTGAGCTA | TCCAGGTTCCAAAATCAGGCT | AATTTCACCCTGACAAA |
| | SEQ ID NO.271 | SEQ ID NO: 283 | SEQ ID NO: 319 |
| 71 | CACAAACCTCTGAGCTA | TCCAGGTTCCAAAATCAGGCT | GGAATTTCACCCTGACA |
| | SEQ ID NO: 271 | SEQ ID NO: 283 | SEQ ID NO: 300 |
| 72 | TCCAGGTTCCAAAATCAG | AAAGTATTCATTCTCTTCATTTTATCCTCGTC | ATTTCACCCTGACAAAGG |
| | SEQ ID NO: 272 | SEQ ID NO: 284 | SEQ ID NO: 320 |
| 73 | TCCAGGTTCCAAAATCAG | AAAGTATTCATTCTCTTCATTTTATCCTCGTC | AATTTCACCCTGACAAAG |
| | SEQ ID NO: 272 | SEQ ID NO: 284 | SEQ ID NO: 310 |
| 74 | TCCAGGTTCCAAAATCAG | AAAGTATTCATTCTCTTCATTTTATCCTCGTC | GGAATTTCACCCTGACAA |
| | SEQ ID NO: 272 | SEQ ID NO: 284 | SEQ ID NO: 307 |
| 75 | TCCAGGTTCCAAAATCAG | AAAGTATTCATTCTCTTCATTTTATCCTCGTC | GAGCTGCCTGGGGAAA |
| | SEQ ID NO: 272 | SEQ ID NO: 284 | SEQ ID NO: 321 |
| 76 | TCCAGGTTCCAAAATCAG | AAGTATTCATTCTCTTCATTTTATCCTCGTCG | ATTTCACCCTGACAAAGG |
| | SEQ ID NO: 272 | SEQ ID NO: 255 | SEQ ID NO: 320 |
| 77 | TCCAGGTTCCAAAATCAG | AAGTATTCATTCTCTTCATTTTATCCTCGTCG | AATTTCACCCTGACAAAG |
| | SEQ ID NO: 272 | SEQ ID NO: 285 | SEQ ID NO: 310 |
| 78 | TCCAGGTTCCAAAATCAG | AAGTATTCATTCTCTTCATTTTATCCTCGTCG | GGAATTTCACCCTGACAA |
| | SEQ ID NO: 272 | SEQ ID NO: 285 | SEQ ID NO: 307 |
| 79 | TCCAGGTTCCAAAATCAG | AAGTATTCATTCTCTTCATTTTATCCTCGTCG | GAGCTGCCTGGGGAAA |
| | SEQ ID NO: 272 | SEQ ID NO: 285 | SEQ ID NO: 321 |
| 80 | TCCAGGTTCCAAAATCAG | ATTCATTCTCTTCATTTTATCCTCGTCGCCC | ATTTCACCCTGACAAAGG |
| | SEQ ID NO: 272 | SEQ ID NO: 286 | SEQ ID NO: 320 |
| 81 | TCCAGGTTCCAAAATCAG | ATTCATTCTCTTCATTTTATCCTCGTCGCCC | AATTTCACCCTGACAAAG |
| | SEQ ID NO: 272 | SEQ ID NO: 286 | SEQ ID NO: 310 |
| 82 | TCCAGGTTCCAAAATCAG | ATTCATTCTCTTCATTTTATCCTCGTCGCCC | GGAATTTCACCCTGACAA |
| | SEQ ID NO: 272 | SEQ ID NO: 286 | SEQ ID NO: 307 |
| 83 | TCCAGGTTCCAAAATCAG | ATTCATTCTCTTCATTTTATCCTCGTCGCCC | GAGCTGCCTGGGGAAA |
| | SEQ ID NO: 272 | SEQ ID NO: 286 | SEQ ID NO: 321 |
| 84 | TCCAGGTTCCAAAATCAG | TCATTCTCTTCATTTTATCCTCGTCGCCC | ATTTCACCCTGACAAAGG |
| | SEQ ID NO: 272 | SEQ ID NO: 287 | SEQ ID NO: 320 |
| 85 | TCCAGGTTCCAAAATCAG | TCATTCTCTTCATTTTATCCTCGTCGCCC | AATTTCACCCTGACAAAG |
| | SEQ ID NO: 272 | SEQ ID NO: 287 | SEQ ID NO: 310 |
| 86 | TCCAGGTTCCAAAATCAG | TCATTCTCTTCATTTTATCCTCGTCGCCC | GGAATTTCACCCTGACAA |
| | SEQ ID NO: 272 | SEQ ID NO: 287 | SEQ ID NO: 307 |
| 87 | TCCAGGTTCCAAAATCAG | TCATTCTCTTCATTTTATCCTCGTCGCCC | GAGCTGCCTGGGGAAA |
| | SEQ ID NO: 272 | SEQ ID NO: 287 | SEQ ID NO: 321 |
| 88 | TCCAGGTTCCAAAATCAG | CATTCTCTTCATTTTATCCTCGTCGCCCC | AATTTCACCCTGACAAAG |
| | SEQ ID NO: 272 | SEQ ID NO: 288 | SEQ ID NO: 310 |
| 89 | TCCAGGTTCCAAAATCAG | CATTCTCTTCATTTTATCCTCGTCGCCCC | GGAATTTCACCCTGACAA |
| | SEQ ID NO: 272 | SEQ ID NO: 288 | SEQ ID NO: 307 |
| 90 | TCCAGGTTCCAAAATCAG | CATTCTCTTCATTTTATCCTCGTCGCCCC | GAGCTGGCTGGGGAAA |
| | SEQ ID NO: 272 | SEQ ID NO: 288 | SEQ ID NO: 321 |
| 91 | TCCAGGTTCCAAAATCAG | ATTCTCTTCATTTTATCCTCGTCGCCCC | AATTTCACCCTGACAAAG |
| | SEQ ID NO: 272 | SEQ ID NO: 289 | SEQ ID NO: 310 |
| 92 | TCCAGGTTCCAAAATCAG | ATTCTCTTCATTTTATCCTCGTCGCCCC | GGAATTTCACCTGACAA |
| | SEQ ID NO: 272 | SEQ ID NO: 289 | SEQ ID NO: 307 |
| 93 | TCCAGGTTCCAAAATCAG | ATTCTCTTCATTTTATCCTCGTCGCCCC | GAGCTGCCTGGGAAA |
| | SEQ ID NO: 272 | SEQ ID NO: 289 | SEQ ID NO: 321 |
| 94 | TCCAGGTTCCAAAATCAG | ATTCTCTTCATTTTATCCTCGTCGCCC | TTTCACCCTGACAAAGG |
| | SEQ ID NO: 272 | SEQ ID NO: 289 | SEQ ID NO: 299 |
| 95 | TCCAGGTTCCAAAATCAG | ATTCTCTTCATTTTATCCTCGTCGCCC | AATTTCACCCTGACAAAG |
| | SEQ ID NO: 272 | SEQ ID NO: 289 | SEQ ID NO: 310 |
| 96 | TCCAGGTTCCAAAATCAG | ATTCTCTTCATTTTATCCTCGTCGCCC | GGAATTTCACCCTGACAA |
| | SEQ ID NO: 272 | SEQ ID NO: 289 | SEQ ID NO: 307 |
| 97 | TCCAGGTTCCAAAATCAG | ATTCTCTTCATTTTATCCTCGTCGCCC | GAGCTGCCTGGGGAAA |
| | SEQ ID NO: 272 | SEQ ID NO: 289 | SEQ ID NO: 321 |
| 98 | TCATTCTCTTCATTTTATCC | TCGTCGCCCCCTTTGTCAGG | AGCTGCCTGGGGAAA |
| | SEQ ID NO: 273 | SEQ ID NO: 290 | SEQ ID NO: 322 |
| 99 | TCATTCTCTTCATTTTATCC | TCGTCGCCCCCTTTGTCAGG | GAGCTCATGGACCTTTT |
| | SEQ ID NO: 273 | SEQ ID NO: 290 | SEQ ID NO: 323 |
| 100 | TCATTCTCTTCATTTTATCC | TCGTCGCCCCCTTTGTCAGG | GGAGCTCATGGACCTTT |
| | SEQ ID NO: 273 | SEQ ID NO: 290 | SEQ ID NO: 324 |
| 101 | CATTCTCTTCATTTTATCC | TCGTCGCCCCCTTTGTCAGG | AGCTCATGGACCTTTT |
| | SEQ ID NO: 274 | SEQ ID NO: 290 | SEQ ID NO: 325 |
| 102 | CATTCTCTTCATTTTATCC | TCGTCGCCCCCTTTGTCAGG | GAGCTCATGGACCTTT |
| | SEQ ID NO: 274 | SEQ ID NO: 290 | SEQ ID NO: 326 |
| 103 | CATTCTCTTCATTTTATCC | TCGTCGCCCCCTTTGTCAGG | GGAGCTCATGGACCTT |
| | SEQ ID NO: 274 | SEQ ID NO: 290 | SEQ ID NO: 327 |
| 104 | CATTCTCTTCATTTTATCC | TCGTCGCCCCCTTTGTCAGG | CATGGAGCTCATGGA |
| | SEQ ID NO: 274 | SEQ ID NO: 290 | SEQ ID NO: 328 |
| 105 | CCTTTGTCAGGGTGAA | TTTCCCCAGGCAGCTCTTTC | AGCTCATGGACCTTTT |
| | SEQ ID NO: 275 | SEQ ID NO: 291 | SEQ ID NO: 325 |
| 106 | CCTTTGTCAGGGTGAA | TTTCCCCAGGCAGCTCTTTC | GAGCTCATGGACCTTTT |
| | SEQ ID NO: 275 | SEQ ID NO: 291 | SEQ ID NO: 323 |
| 107 | CCTTTGTCAGGGTGAA | TTTCCCCAGGCAGCTCTTTC | GGAGCTCATGGACCTT |
| | SEQ ID NO: 275 | SEQ ID NO: 291 | SEQ ID NO: 327 |
| 108 | CTTTGTCAGGGTGAAAT | TTTCCCCAGGCAGCTCTTTC | GAGCTCATGGACCTTTT |
| | SEQ ID NO: 276 | SEQ ID NO: 291 | SEQ ID NO: 323 |
| 109 | CTTTGTCAGGGTGAAAT | TTTCCCCAGGCAGCTCTTTC | GAGCTCATGGACCTTT |
| | SEQ ID NO: 276 | SEQ ID NO: 291 | SEQ ID NO: 326 |
| 110 | TTTGTCAGGGTGAAAT | TTTCCCCAGGCAGCTCTTTC | AGCTCATGGACCTTTT |
| | SEQ ID NO: 277 | SEQ ID NO: 291 | SEQ ID NO: 325 |
| 111 | TTTGTCAGGGTGAAAT | TTTCCCCAGGCAGCTCTTTC | GAGCTCATGGACCTTT |
| | SEQ ID NO: 277 | SEQ ID NO: 291 | SEQ ID NO: 326 |
| 112 | TTTGTCAGGGTGAAAT | TTTCCCCAGGCAGCTCTTTC | GGAGCTGATGGACCTT |
| | SEQ ID NO: 277 | SEQ ID NO: 291 | SEQ ID NO: 327 |
| 113 | TCAGGGTGAAATTCCT | TTTCCCCAGGCAGCTCTTTC | AGCTCATGGACCTTTT |
| | SEQ ID NO: 278 | SEQ ID NO: 291 | SEQ ID NO: 325 |
| 114 | TCAGGGTGAAATTCCT | TTTCCCCAGGCAGCTCTTTC | GAGCTCATGGACCTTTT |
| | SEQ ID NO: 278 | SEQ ID NO: 291 | SEQ ID NO: 323 |
| 115 | TCAGGGTGAAATTCCT | TTTCCCCAGGCAGCTCTTTC | GAGCTCATGGACCTTT |
| | SEQ ID NO: 278 | SEQ ID NO: 291 | SEQ ID NO: 326 |
| 116 | TCAGGGTGAAATTCCT | TTTCCCCAGGCAGCTCTTTC | GGAGCTCATGGACCTT |
| | SEQ ID NO: 278 | SEQ ID NO: 291 | SEQ ID NO: 327 |
| 117 | CAGGGTGAAATTCCTTA | TTTCCCCAGGCAGCTCTTTC | AGCTCATGGACCTTTT |
| | SEQ ID NO: 279 | SEQ ID NO: 291 | SEQ ID NO: 325 |
| 118 | CAGGGTGAAATTCCTTA | TTTCCCCAGGCAGCTCTTTC | GAGCTCATGGACCTTT |
| | SEQ ID NO: 279 | SEQ ID NO: 291 | SEQ ID NO: 326 |
| 119 | CAGGGTGAAATTCCTTA | TTTCCCCAGGCAGCTCTTTC | GGAGCTCATGGACCTT |
| | SEQ ID NO: 279 | SEQ ID NO: 291 | SEQ ID NO: 327 |
| 120 | CAGGGTGAAATTCCTTA | TTTCCCCAGGCAGCTCTTTC | CATGGAGCTCATGGA |
| | SEQ ID NO: 279 | SEQ ID NO: 291 | SEQ ID NO: 328 |
| 121 | AGGGTGAAATTCCTTAC | TTTCCCCAGGCAGCTCTTTC | AGCTCATGGACCTTTT |
| | SEQ ID NO: 280 | SEQ ID NO: 291 | SEQ ID NO: 325 |
| 122 | AGGGTGAAATTCCTTAC | TTTCCCCAGGCAGCTCTTTC | GAGCTCATGGACCTTT |
| | SEQ ID NO: 280 | SEQ ID NO: 291 | SEQ ID NO: 326 |
| 123 | AGGGTGAAATTCCTTAC | TTTCCCCAGGCAGCTCTTTC | GGAGCTCATGGACCTT |
| | SEQ ID NO: 280 | SEQ ID NO: 291 | SEQ ID NO: 327 |
| 124 | AGGGTGAAATTCCTTAC | TTTCCCCAGGCAGCTCTTTC | CATGGAGCTCATGGA |
| | SEQ ID NO: 280 | SEQ ID NO: 291 | SEQ ID NO: 328 |
| 125 | GGGTGAAATTCCTTACA | TTTCCCCAGGCAGCTCTTTC | AGCTCATGGACCTTTT |
| | SEQ ID NO: 281 | SEQ ID NO: 291 | SEQ ID NO: 325 |
| 126 | GGGTGAAATTCCTTACA | TTTCCCCAGGCAGCTCTTTC | GAGCTCATGGACCTTTT |
| | SEQ ID NO: 281 | SEQ ID NO: 291 | SEQ ID NO: 323 |
| 127 | GGGTGAAATTCCTTACA | TTTCCCCAGGCAGCTCTTTC | GGAGCTCATGGACCTT |
| | SEQ ID NO: 281 | SEQ ID NO: 291 | SEQ ID NO: 327 |
| 128 | GGGTGAAATTCCTTACA | TTTCCCCAGGCAGCTCTTTC | CATGGAGCTCATGGA |
| | SEQ ID NO: 281 | SEQ ID NO: 291 | SEQ ID NO: 328 |
| 129 | CACAAACCTCTGAGCTA | TCCAGGTTCCAAAATCAGGCT | GGGGCGACGAGGATA |
| | SEQ ID NO: 329 | SEQ ID NO: 332 | SEQ ID NO: 331 |
| 130 | TCCAGGTTCCAAAATCA | TCTTCATTTTATCCTCGTCGCCC | GAGCTGCCTGGGGAAA |
| | SEQ ID NO: 330 | SEQ ID NO: 333 | SEQ ID NO: 321 |
| 131 | CACAAACCTCTGAGCTA | AAGATTCAGCAGCTACCGCAGCCC | GGGGCGACGAGGATA |
| | SEQ ID NO: 329 | SEQ ID NO: 334 | SEQ ID NO: 331 |

A PCR primer pair for amplifying BK virus DNA can comprise any one of the following pairs of primer sequences (1) SEQ ID NOS: 256 and 292; (2) SEQ ID NOS: 257 and 292; (3) SEQ ID NOS: 257 and 293; (4) SEQ ID NOS: 258 and 292; (5) SEQ ID NOS: 259 and 292; (6) SEQ ID NOS: 260 and 292; (7) SEQ ID NOS: 261 and 292; (8) SEQ ID NOS: 262 and 292; (9) SEQ ID NOS: 263 and 293; (10) SEQ ID NOS: 264 and 294; (11) SEQ ID NOS: 264 and 295; (12) SEQ ID NOS: 264 and 296; (13) SEQ ID NOS: 264 and 297; (14) SEQ ID NOS: 264 and 298; (15) SEQ ID NOS: 264 and 299; (16) SEQ ID NOS: 264 and 300; (17) SEQ ID NOS: 265 and 294; (18) SEQ ID NOS: 265 and 301; (19) SEQ ID NOS: 265 and 302; (20) SEQ ID NOS: 265 and 303; (21) SEQ ID NOS: 265 and 304; (22) SEQ ID NOS: 265 and 305; (23) SEQ ID NOS: 265 and 299; (24) SEQ ID NOS: 265 and 306; (25) SEQ ID NOS: 265 and 307; (26) SEQ ID NOS: 266 and 294; (27) SEQ ID NOS: 266 and 301; (28) SEQ ID NOS: 266 and 296; (29) SEQ ID NOS: 266 and 303; (30) SEQ ID NOS: 266 and 298; (31) SEQ ID NOS: 266 and 308; (32) SEQ ID NOS: 266 and 309; (33) SEQ ID NOS: 266 and 300; (34) SEQ ID NOS: 267 and 294; (35) SEQ ID NOS: 267 and 301; (36) SEQ ID NOS: 267 and 296; (37) SEQ ID NOS: 267 and 303; (38) SEQ ID NOS: 267 and 298; (39) SEQ ID NOS: 267 and 305; (40) SEQ ID NOS: 267 and 299; (41) SEQ ID NOS: 267 and 310; (42) SEQ ID NOS: 267 and 300; (43) SEQ ID NOS: 268 and 308; (44) SEQ ID NOS: 268 and 299; (45) SEQ ID NOS: 268 and 310; (46) SEQ ID NOS: 268 and 300; (47) SEQ ID NOS: 269 and 311; (48) SEQ ID NOS: 269 and 312; (49) SEQ ID NOS: 269 and 313; (50) SEQ ID NOS: 269 and 314; (51) SEQ ID NOS: 269 and 315; (52) SEQ ID NOS: 269 and 316; (53) SEQ ID NOS: 269 and 306; (54) SEQ ID NOS: 270 and 294; (55) SEQ ID NOS: 270 and 317; (56) SEQ ID NOS:270 and 303; (57) SEQ ID NOS: 270 and 298; (58) SEQ ID NOS: 270 and 308; (59) SEQ ID NOS: 270 and 309; (60) SEQ ID NOS: 270 and 310; (61) SEQ ID NOS: 270 and 300; (62) SEQ ID NOS:271 and 294; (63) SEQ ID NOS: 271 and 301; (64) SEQ ID NOS: 271 and 296; (65) SEQ ID NOS: 271 and 303; (66) SEQ ID NOS: 271 and 298; (67) SEQ ID NOS: 271 and 305; (68) SEQ ID NOS: 271 and 318; (69) SEQ ID NOS: 271 and 309; (70) SEQ ID NOS: 271 and 319; (71) SEQ ID NOS: 271 and 300; (72) SEQ ID NOS: 272 and 320; (73) SEQ ID NOS: 272 and 310; (74) SEQ ID NOS: 272 and 307; (75) SEQ ID NOS: 272 and 321; (76) SEQ ID NOS: 272 and 299; (77) SEQ ID NOS: 273 and 322; (78) SEQ ID NOS: 273 and 323; (79) SEQ ID NOS: 273 and 324; (80) SEQ ID NOS: 274 and 325; (81) SEQ ID NOS: 274 and 326; (82) SEQ ID NOS: 274 and 327; (83) SEQ ID NOS: 274 and 328; (84) SEQ ID NOS: 275 and 325; (85) SEQ ID NOS: 275 and 323; (86) SEQ ID NOS: 275 and 327; (87) SEQ ID NOS: 276 and 323; (88) SEQ ID NOS: 276 and 326; (89) SEQ ID NOS: 277 and 325; (90) SEQ ID NOS: 277 and 326; (91) SEQ ID NOS: 277 and 327; (92) SEQ ID NOS: 278 and 325; (93) SEQ ID NOS: 278 and 323; (94) SEQ ID NOS: 278 and 326; (95) SEQ ID NOS: 278 and 327 (96) SEQ ID NOS: 279 and 325; (97) SEQ ID NOS: 279 and 326; (98) SEQ ID NOS: 279 and 327; (99) SEQ ID NOS: 279 and 328; (100) SEQ ID NOS: 280 and 325; (101) SEQ ID NOS: 280 and 326; (102) SEQ ID NOS: 280 and 327; (103) SEQ ID NOS: 280 and 328; (104) SEQ ID NOS: 281 and 325; (105) SEQ ID NOS: 281 and 323; (106) SEQ ID NOS: 281 and 327; (107) SEQ ID NOS: 281 and 328; (108) SEQ ID NOS: 329 and 331; (109) SEQ ID NOS: 330 and 321; and (110) SEQ ID NOS: 329 and 331.

The preceding numbering of the one-hundred ten pairs of primers does not correspond exactly to the “Group” numbering scheme in Table 4 because certain groups use the same primer pair, but different internal probes. For example, Groups 74, 78, 82, 86, 89, 92 and 96 each employ the forward primer of SEQ ID NO:272 and the reverse primer of SEQ ID NO:307, but six different internal probes, e.g., SEQ, D NOS: 284-289. Accordingly, primer pair “(74)” of the preceding passage implies any one of Groups 74, 78, 82, 86, 89, 92 and 96 of Table 4. Likewise, Groups 73, 77, 81, 85, 88, 91, and 95 of Table 4 each employ SEQ ID NOS: 272 and 310 as primers, but six different internal probes, e.g., SEQ ID NOS: 284-289. Groups 73, 77, 81, 85, 88, 91, and 95 are depicted in primer pair “(73)” in the preceding passage.

The probe of SEQ ID NO:282 can be used to hybridize to the product amplified with the primer pair of Groups 1-9 depicted in Table 4. The probe of SEQ ID NO:283 can be used to hybridize to the product amplified with the primer pair of Groups 10-71 depicted in Table 4. The probe of SEQ ID NO:284 can be used to hybridize to the product amplified with the primer pair of Groups 72-75 depicted in Table 4. The probe of SEQ ID NO:285 can be used to hybridize to the product amplified with the primer pair of Groups 76-79 depicted in Table 4. The probe of SEQ ID NO:286 can be used to hybridize to the product amplified with the primer pair of Groups 80-83 depicted in Table 4. The probe of SEQ ID NO:287 can be used to hybridize to the product amplified with the primer pair of Groups 84-87 depicted in Table 4. The probe of SEQ ID NO:288 can be used to hybridize to the product amplified with the primer pair of Groups 88-90 depicted in Table 4. The probe of SEQ ID NO:289 can be used to hybridize to the product amplified with the primer pair of Groups 91-97 depicted in Table 4. The probe of SEQ ID NO:290 can be used to hybridize to the product amplified with the primer pair of Groups 98-104 depicted in Table 4. The probe of SEQ ID NO:291 can be used to hybridize to the product amplified with the primer pair of Groups 105-128 depicted in Table 4. The probe of SEQ ID NO:332 can be used to hybridize to the product amplified with the primer pair of Group 129 depicted in Table 4. The probe of SEQ ID NO:333 can be used to hybridize to the product amplified with the primer pair of Group 130 depicted in Table 4. In Table 4, SEQ ID NO:334 is an internal control probe i.e., not a BKV probe, for binding to an internal control target sequence comprising SEQ ID NO:336 (GGGCTGCGGTAGCTGCTGAATCTT) for monitoring (1) the efficiency of extraction of nucleic acids from a sample; and (2) the amplification step of PCR

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

What is claimed is:

1. An isolated polynucleotide comprising a nucleotide sequence selected from the group consisting of: SEQ ID NOS: 1-334.

2. A PCR primer pair for amplifying BK virus or one or more variants thereof, comprising a pair of sequences selected from the group consisting of: SEQ ID NOS: 1 and 3; (2) SEQ ID NOS: 6 and 3; (3) SEQ ID NOS: 7 and 3; (4) SEQ ID NOS: 8 and 3; (5) SEQ ID NOS: 9 and 3; (6) SEQ ID NOS: 10 and 12; (7) SEQ ID NOS: 13 and 12; (8) SEQ ID NOS: 15 and 17; (9) SEQ ID NOS: 15 and 19; (10) SEQ ID NOS: 20 and 21; (I 1) SEQ ID NOS: 15 and 22; (12) SEQ ID NOS: 23 and 25; (13) SEQ ID NOS: 26 and 25; (14) SEQ ID NOS: 28 and 29; (15) SEQ ID NOS: 15 and 30; (16) SEQ ID NOS: 28 and 31; (17) SEQ ID NOS: 15 and 32; (18) SEQ ID NOS: 20 and 32; (19) SEQ ID NOS: 10 and 33; (20) SEQ ID NOS: 10 and 34; (21) SEQ ID NOS: 23 and 35, (22) SEQ ID NOS: 28 and 36; (23) SEQ ID NOS: 37 and 39; (24) SEQ ID NOS: 37 and 36; (25) SEQ ID NOS: 28 and 33; (26) SEQ ID NOS: 13 and 32; (27) SEQ ID NOS: 42 and 43; (28) SEQ ID NOS: 28 and 44; (29) SEQ ID NOS: 10 and 45; (30) SEQ ID NOS: 15 and 46; (31) SEQ ID NOS: 10 and 47; (32) SEQ ID NOS: 20 and 48; (33) SEQ ID NOS: 10 and 49; (34) SEQ ID NOS: 15 and 50; (35) SEQ ID NOS: 10 and 51; (36) SEQ ID NOS: 52 and 54; (37) SEQ ID NOS: 20 and 47; (38) SEQ ID NOS: 10 and 49; (39) SEQ ID NOS: 55 and 57; (40) SEQ ID NOS: 58 and 19; (41) SEQ ID NOS: 60 and 22; (42) SEQ ID NOS: 55 and 62; (43) SEQ ID NOS: 58 and 34; (44) SEQ ID NOS: 26 and 66; (45) SEQ ID NOS: and 67; (46) SEQ ID NOS: 68 and 50; (47) SEQ ID NOS: 20 and 70; (48) SEQ ID NOS: 10 and 72; (49) SEQ ID NOS: 55 and 73; (50) SEQ ID NOS: 55 and 74; (51) SEQ ID NOS: 37 and 75; (52) SEQ ID NOS: 76 and 25; (53) SEQ ID NOS: 55 and 78; (54) SEQ ID NOS: 26 and 79; (55) SEQ ID NOS: 80 and 82; (56) SEQ ID NOS: 58 and 84; (57) SEQ ID NOS: 76 and 29; (58) SEQ ID NOS: 15 and 21; (59) SEQ ID NOS: 23 and 35; (60) SEQ ID NOS: 87 and 25; (61) SEQ ID NOS: 88 and 25; (62) SEQ ID NOS: 10 and 89; (63) SEQ ID NOS: 10 and 90; (64) SEQ ID NOS: 20 and 91; (65) SEQ ID NOS: 55 and 78; (66) SEQ ID NOS: 92 and 94; (67) SEQ ID NOS: 95 and 57; (68) SEQ ID NOS: 97 and 94; (69) SEQ ID NOS: 100 and 94; (70) SEQ ID NOS: 97 and 103; (71) SEQ ID NOS: 95 and 12; (72) SEQ ID NOS: 104 and 94; (73) SEQ ID NOS: 97 and 105; (74) SEQ ID NOS: 97 and 21; (75) SEQ ID NOS: 106 and 94; (76) SEQ ID NOS: 107 and 103; (77) SEQ ID NOS: 109 and 103; (78) SEQ ID NOS: 111 and 112; (79) SEQ ID NOS: 113 and 94; (80) SEQ ID NOS: 114 and 103; (81) SEQ ID NOS: 115 and 116; (82) SEQ ID NOS: 117 and 94; (83) SEQ ID NOS: 109 and 105; (84) SEQ ID NOS: 118 and 94; (85) SEQ ID NOS: 119 and 103; (86) SEQ ID NOS: 120 and 103; (87) SEQ ID NOS: and 103; (88) SEQ ID NOS: 123 and 105; (89) SEQ ID NOS: 125 and 103; (90) SEQ ID NOS: 126 and 12; (91) SEQ ID NOS: 128 and 62; (92) SEQ ID NOS: 129 and 105; (93) SEQ ID NOS: 130 and 12; (94) SEQ ID NOS: 131 and 94; (95) SEQ ID NOS: 132 and 94; (96) SEQ ID NOS: 107 and 12; (97) SEQ ID NOS: 97 and 57; and (98) SEQ ID NOS: 98 and 57.

3. A PCR primer pair for amplifying BK virus or one or more variants thereof, comprising a pair of sequences selected from the group consisting of: (1) SEQ ID NOS: 147 and 234; (2) SEQ ID NOS: 146 and 217; (3) SEQ ID NOS: 148 and 225; (4) SEQ ID NOS: 172 and 250: (5) SEQ ID NOS: 138 and 216; (6) SEQ ID NOS: 139 and 221; (7) SEQ ID NOS: 150 and 227; (8) SEQ ID NOS: 145 and 233; (9) SEQ ID NOS: 142 and 226; (10) SEQ ID NOS: 170 and 248; (11) SEQ 11) NOS: 151 and 230: (12) SEQ ID NOS: 158 and 241; (13) SEQ ID NOS: 150 and 238; (14) SEQ ID NOS: 147 and 218; (15) SEQ ID NOS: 146 and 228; (16) SEQ ID NOS: 136 and 237; (17) SEQ ID NOS: 172 and 249; (18) SEQ ID NOS: 138 and 223; (19) SEQ ID NOS: 143 and 219; (20) SEQ ID NOS: 157 and 243; (21) SEQ ID NOS: 139 and 223; (22) SEQ ID NOS: 150 and 235; (23) SEQ ID NOS: 150 and 232; (24) SEQ ID NOS: 147 and 233; (25) SEQ ID NOS: 146 and 237; (26) SEQ ID NOS: 142 and 223; (27) SEQ ID NOS: 148 and 215; (28) SEQ ID NOS: 139 and 230; (29) SEQ ID NOS: 162 and 242; (30) SEQ ID NOS: 137 and 215; (31) SEQ ID NOS: 140 and 222; (32) SEQ ID NOS: 166 and 241; (33) SEQ ID NOS: 156 and 234; (34) SEQ ID NOS: 143 and 221; (35) SEQ ID NOS: 165 and 242; (36) SEQ ID NOS: 149 and 230; (37) SEQ ID NOS: 141 and 223; (38) SEQ ID NOS: 147 and 236; (39) SEQ ID NOS: 146 and 229; (40) SEQ ID NOS: 168 and 251; (41) SEQ ID NOS: 171 and 248; (42) SEQ ID NOS: 138 and 220; (43) SEQ ID NOS: 160 and 241; (44) SEQ ID NOS: 164 and 252; (45) SEQ ID NOS: 139 and 231; (46) SEQ ID NOS: 150 and 223; (47) SEQ ID NOS: 144 and 233; (48) SEQ ID NOS: 170 and 252; (49) SEQ ID NOS: 163 and 242; (50) SEQ ID NOS: 150 and 228; (51) SEQ ID NOS: 147 and 226; (52) SEQ ID NOS: 140 and 217; (53) SEQ ID NOS: 172 and 247; (54) SEQ ID NOS: 143 and 230; (55) SEQ ID NOS: 139 and 224; (56) SEQ ID NOS: 150 and 235; (57) SEQ ID NOS: 184 and 253; (58) SEQ ID NOS: 168 and 255; (59) SEQ ID NOS: 175 and 255; (60) SEQ ID NOS: 173 and 253; (61) SEQ ID NOS: 164 and 255; (62) SEQ ID NOS: 177 and 253; (63) SEQ ID NOS: 169 and 253; (64) SEQ ID NOS: 183 and 255; (65) SEQ ID NOS: 172 and 245; (66) SEQ ID NOS: 166 and 254; (67) SEQ ID NOS: 178 and 254; (68) SEQ ID NOS: 165 and 255; (69) SEQ ID NOS: 174 and 253; (70) SEQ ID NOS: 171 and 254; (71) SEQ ID NOS: 160 and 255; (72) SEQ ID NOS: 172 and 246; (73) SEQ ID NOS: 175 and 254; (74) SEQ ID NOS: 161 and 253; (75) SEQ ID NOS: 159 and 255; (76) SEQ ID NOS: 167 and 253; (77) SEQ ID NOS: 183 and 254; (78) SEQ ID NOS: 181 and 255; (79) SEQ ID NOS: 150 and 240; (80) SEQ ID NOS: 179 and 253; (81) SEQ ID NOS: 172 and 254; (82) SEQ ID NOS: 176 and 253; (83) SEQ ID NOS: 183 and 253; (84) SEQ ID NOS: 166 and 255; (85) SEQ ID NOS: 154 and 239; (86) SEQ ID NOS: 178 and 253; (87) SEQ ID NOS: 153 and 239; (88) SEQ ID NOS: 152 and 239; (89) SEQ ID NOS: 185 and 254; (90) SEQ ID NOS: 171 and 255; (91) SEQ ID NOS: 155 and 239; (92) SEQ ID NOS: 182 and 255; (93) SEQ ID NOS:180 and 254; (94) SEQ ID NOS: 172 and 244; and (95) SEQ ID NOS: 183 and 253.

4. A PCR primer pair for amplifying BK virus or one or more variants thereof, comprising a pair of sequences selected from the group consisting of: (1) SEQ ID NOS: 256 and 292; (2) SEQ ID NOS: 257 and 292; (3) SEQ ID NOS: 257 and 293; (4) SEQ ID NOS: 258 and 292; (5) SEQ ID NOS: 259 and 292; (6) SEQ ID NOS: 260 and 292; (7) SEQ ID NOS: 261 and 292; (8) SEQ ID NOS: 262 and 292; (9) SEQ ID NOS: 263 and 293, (10) SEQ ID NOS: 264 and 294; (11) SEQ ID NOS: 264 and 295; (12) SEQ ID NOS: 264 and 296; (13) SEQ ID NOS: 264 and 297; (14) SEQ ID NOS: 264 and 298; (15) SEQ ID NOS: 264 and 299; (16) SEQ ID NOS: 264 and 300; (17) SEQ ID NOS: 265 and 294; (18) SEQ ID NOS: 265 and 301; (19) SEQ ID NOS: 265 and 302; (20) SEQ ID NOS: 265 and 303; (21) SEQ ID NOS: 265 and 304; (22) SEQ ID NOS: 265 and 305; (23) SEQ ID NOS: 265 and 299; (24) SEQ ID NOS: 265 and 306; (25) SEQ ID NOS: 265 and 307; (26) SEQ ID NOS: 266 and 294; (27) SEQ ID NOS: 266 and 301; (28) SEQ ID NOS: 266 and 296; (29) SEQ ID NOS: 266 and 303; (30) SEQ ID NOS: 266 and 298; (31) SEQ ID NOS: 266 and 308; (32) SEQ ID NOS: 266 and 309; (33) SEQ ID NOS: 266 and 300; (34) SEQ ID NOS: 267 and 294; (35) SEQ ID NOS: 267 and 301; (36) SEQ ID NOS: 267 and 296; (37) SEQ ID NOS: 267 and 303; (38) SEQ ID NOS: 267 and 298; (39) SEQ ID NOS: 267 and 305Q(40) SEQ ID NOS: 267 and 299; (41) SEQ ID NOS: 267 and 310; (42) SEQ ID NOS: 267 and 300; (43) SEQ ID NOS: 268 and 308; (44) SEQ ID NOS: 268 and 299; (45) SEQ ID NOS: 268 and 310; (46) SEQ ID NOS: 268 and 300; (47) SEQ ID NOS: 269 and 311; (48) SEQ ID NOS: 269 and 312; (49) SEQ ID NOS: 269 and 313 (50) SEQ ID NOS: 269 and 314; (51) SEQ ID NOS: 269 and 315; (52) SEQ ID NOS: 269 and 316; (53) SEQ ID NOS: 269 and 306; (54) SEQ ID NOS: 270 and 294; (55) SEQ ID NOS: 270 and 317; (56) SEQ ID NOS:270 and 303; (57) SEQ ID NOS: 270 and 298; (58) SEQ ID NOS: 270 and 308 (59) SEQ ID NOS: 270 and 309; (60) SEQ ID NOS: 270 and 310; (61) SEQ ID NOS: 270 and 300; (62) SEQ ID NOS: 271 and 294; (63) SEQ ID NOS: 271 and 301; (64) SEQ ID NOS: 271 and 296; (65) SEQ ID NOS: 271 and 303; (66) SEQ ID NOS: 271 and 298; (67) SEQ ID NOS: 271 and 305; (68) SEQ ID NOS: 271 and 318; (69) SEQ ID NOS: 271 and 309; (70) SEQ ID NOS: 271 and 319; (71) SEQ ID NOS: 271 and 300; (72) SEQ ID NOS: 272 and 320; (73) SEQ ID NOS: 272 and 310; (74) SEQ ID NOS: 272 and 307; (75) SEQ ID NOS: 272 and 321; (76) SEQ ID NOS: 272 and 299; (77) SEQ ID NOS: 273 and 322; (78) SEQ ID NOS: 273 and 323; (79) SEQ ID NOS: 273 and 324; (80) SEQ ID NOS: 274 and 325; (81) SEQ ID NOS: 274 and 326; (82) SEQ ID NOS: 274 and 327; (83) SEQ ID NOS: 274 and 328; (84) SEQ ID NOS: 275 and 325; (85) SEQ ID NOS: 275 and 323; (86) SEQ ID NOS: 275 and 327; (87) SEQ ID NOS: 276 and 323; (88) SEQ ID NOS: 276 and 326; (89) SEQ ID NOS: 277 and 325; (90) SEQ ID NOS: 277 and 326; (91) SEQ ID NOS: 277 and 327; (92) SEQ ID NOS 278 and 325; (93) SEQ ID NOS: 278 and 323; (94) SEQ ID NOS: 278 and 326; (95) SEQ ID NOS: 278 and 327; (96) SEQ ID NOS: 279 and 325; (97) SEQ ID NOS: 279 and 326; (98) SEQ ID NOS: 279 and 327; (99) SEQ ID NOS: 279 and 328; (1 00) SEQ ID NOS: 280 and 325; (101) SEQ ID NOS: 280 and 326; (102) SEQ ID NOS: 280 and 327; (103) SEQ ID NOS: 280 and 328; (104) SEQ ID NOS: 281 and 325; (105) SEQ ID NOS: 281 and 323; (106) SEQ ID NOS: 281 and 327; (107) SEQ ID NOS: 281 and 328; (108) SEQ ID NOS: 329 and 331; (109) SEQ ID NOS: 330 and 321; and (110) SEQ ID NOS: 329 and 331.

5. A polynucleotide probe for binding to BK virus DNA, comprising a nucleotide sequence selected from the group consisting of SEQ ID NOS: 2, 4, 5, 11, 14, 16, 18, 24, 27, 38, 40, 41, 53, 56, 59, 617 637 647 65, 69, 71, 77, 81, 83, 85, 86, 93, 96, 99, 101, 102, 108, 110, 122, 124, 127, 133-134, 186-214, 282-291 332-333.

6. The polynucleotide probe of claim 5, wherein the probe does not cross react with an identified nucleic acid sequence of a polyoma virus with high efficiency.

7. The polynucleotide probe of claim 5, wherein the probe is labeled for quantitating BK virus.

8. The polynucleotide probe of claim 5, wherein the probe is labeled for detecting BK virus.

9. The polynucleotide probe of claim 7 or 8, wherein the probe comprises a detectable moiety selected from the group consisting of: a fluorescent label, a chemiluminescent label, a radioactive label, a quenching molecule biotin and gold.

10. A method for detecting BK virus in a sample comprising:
(1) contacting at least one forward and reverse primer pair listed in Tables 2, 3 or 4 to a sample;
(2) conducting a polymerase chain reaction; and
(3) detecting and/or quantitating the generation of an amplified PCR product,
wherein the generation of an amplified PCR product indicates the presence of BK virus in the sample.

11. The method of claim 10, wherein the forward and reverse primer pair are selected from the group consisting of: (1) SEQ ID NOS: 1 and 3; (2) SEQ ID NOS: 6 and 3; (3) SEQ ID NOS: 7 and 3; (4) SEQ ID NOS: 8 and 3; (5) SEQ ID NOS: 9 and 3; (6) SEQ ID NOS: 10 and 12; (7) SEQ ID NOS: 13 and 12; (8) SEQ ID NOS: 15 and 17; (9) SEQ ID NOS: 15 and 19; (10) SEQ ID NOS: 20 and 21; (11) SEQ ID NOS: 15 and 22; (12) SEQ ID NOS: 23 and 25; (13) SEQ ID NOS: 26 and 25; (14) SEQ ID NOS: 28 and 29; (15) SEQ ID NOS: 15 and 30; (16) SEQ ID NOS: 28 and 31; (17) SEQ ID NOS: 15 and 32; (18) SEQ ID NOS: 20 and 32; (19) SEQ ID NOS: 10 and 33; (20) SEQ ID NOS: 10 and 34; (21) SEQ ID NOS: 23 and 35; (22) SEQ ID NOS: 28 and 36; (23) SEQ ID NOS: 37 and 39; (24) SEQ ID NOS: 37 and 36; (25) SEQ ID NOS: 28 and 33; (26) SEQ ID NOS: 13 and 32; (27) SEQ ID NOS: 42 and 43; (28) SEQ ID NOS: 28 and 44; (29) SEQ ID NOS: 10 and 45; (30) SEQ ID NOS: 15 and 46; (31) SEQ ID NOS: 10 and 47; (32) SEQ ID NOS: 20 and 48; (33) SEQ ID NOS: 10 and 49; (34) SEQ ID NOS: 15 and 50; (35) SEQ ID NOS: 10 and 51; (36) SEQ ID NOS: 52 and 54; (37) SEQ ID NOS: 20 and 47; (38) SEQ ID NOS: 10 and 49; (39) SEQ ID NOS: 55 and 57; (40) SEQ ID NOS: 58 and 19; (41) SEQ ID NOS: 60 and 22; (42) SEQ ID NOS: 55 and 62; (43) SEQ ID NOS: 58 and 34; (44) SEQ ID NOS: 26 and 66; (45) SEQ ID NOS: 37 and 67; (46) SEQ ID NOS: 68 and 50; (47) SEQ ID NOS: 20 and 70; (48) SEQ ID NOS: 10 and 72; (49) SEQ ID NOS: 55 and 73; (50) SEQ ID NOS: 55 and 74; (51) SEQ ID NOS: 37 and 75; (52) SEQ ID NOS: 76 and 25; (53) SEQ ID NOS: 55 and 78; (54) SEQ ID NOS: 26 and 79; (55) SEQ ID NOS: 80 and 82; (56) SEQ ID NOS: 58 and 84; (57) SEQ ID NOS: 76 and 29; (58) SEQ ID NOS: 15 and 21; (59) SEQ ID NOS: 23 and 35; (60) SEQ ID NOS: 87 and 25; (61) SEQ ID NOS: 88 and 25; (62) SEQ ID NOS: 10 and 89; (63) SEQ ID NOS: 10 and 90; (64) SEQ ID NOS: 20 and 91; (65) SEQ ID NOS: 55 and 78; (66) SEQ ID NOS: 92 and 94; (67) SEQ ID NOS: 95 and 57; (68) SEQ ID NOS: 97 and 94; (69) SEQ ID NOS: 100 and 94; (70) SEQ ID NOS: 97 and 103; (71) SEQ ID NOS: 95 and 12; (72) SEQ ID NOS: 104 and 94; (73) SEQ ID NOS: 97 and 105; (74) SEQ ID NOS: 97 and 21; (75) SEQ ID NOS: 106 and 94; (76) SEQ ID NOS: 107 and 103; (77) SEQ ID NOS: 109 and 103; (78) SEQ ID NOS: 111 and 112; (79) SEQ ID NOS: 113 and 94; (80) SEQ ID NOS: 114 and 103; (81) SEQ ID NOS: 115 and 116; (82) SEQ ID NOS: 117 and 94; (83) SEQ ID NOS: 109 and 105; (84) SEQ ID NOS: 118 and 94; (85) SEQ ID NOS: 119 and 103; (86) SEQ ID NOS: 120 and 103; (87) SEQ ID NOS: 121 and 103; (88) SEQ ID NOS: 123 and 105; (89) SEQ ID NOS: 125 and 103; (90) SEQ ID NOS: 126 and 12 (91) SEQ ID NOS: 128 and 62; (92) SEQ ID NOS: 129 and 105; (93) SEQ ID NOS: 130 and 12; (94) SEQ ID NOS: 131 and 94; (95) SEQ ID NOS: 132 and 94, (96) SEQ ID NOS: 107 and 12; (97) SEQ ID NOS: 97 and 57; and (98) SEQ ID NOS: 98 and 57.

12. The method of claim 10, wherein the forward and reverse primer pair are selected from the group consisting of: (1) SEQ ID NOS: 147 and 234; (2) SEQ ID NOS: 146 and 217; (3) SEQ ID NOS: 148 and 225; (4) SEQ ID NOS: 172 and 250; (5) SEQ ID NOS: 138 and 216; (6) SEQ ID NOS: 139 and 221; (7) SEQ ID NOS: 150 and 227; (8) SEQ ID NOS: 145 and 233; (9) SEQ ID NOS: 142 and 226; (10) SEQ ID NOS: 170 and 248; (11) SEQ ID NOS: 151 and 230; (12) SEQ ID NOS: 158 and 241; (13) SEQ ID NOS: 150 and 238; (14) SEQ ID NOS: 147 and 218; (15) SEQ ID NOS: 146 and 228; (16) SEQ ID NOS: 136 and 237; (17) SEQ ID NOS: 172 and 249; (18) SEQ ID NOS: 138 and 223; (19) SEQ ID NOS: 143 and 219; (20) SEQ ID NOS: 157 and 243; (21) SEQ ID NOS: 139 and 223; (22) SEQ ID NOS: 150 and 235; (23) SEQ ID NOS: 150 and 232; (243 SEQ ID NOS: 147 and 233; (25) SEQ ID NOS: 146 and 237; (26) SEQ ID NOS: 142 and 223; (27) SEQ ID NOS: 148 and 215; (28) SEQ ID NOS: 139 and 230; (29) SEQ ID NOS: 162 and 242; (30) SEQ ID NOS: 137 and 215; (31) SEQ ID NOS: 140 and 222; (32) SEQ ID NOS: 166 and 241; (33) SEQ ID NOS: 156 and 234; (34) SEQ ID NOS: 143 and 221; (35) SEQ ID NOS: 165 and 242; (36) SEQ ID NOS: 149 and 230; (37) SEQ ID NOS: 141 and 223; (38) SEQ ID NOS: 147 and 236; (39) SEQ ID NOS: 146 and 229; (40) SEQ ID NOS: 168 and 251; (41) SEQ ID NOS: 171 and 248; (42) SEQ ID NOS: 138 and 220; (43) SEQ ID NOS: 160 and 241; (44) SEQ ID NOS: 164 and 252; (45) SEQ ID NOS: 139 and 231; (46) SEQ ID NOS: 150 and 223; (47) SEQ ID NOS: 144 and 233; (48) SEQ ID NOS: 170 and 252; (49) SEQ ID NOS: 163 and 242; (50) SEQ ID NOS: 150 and 228; (51) SEQ ID NOS: 147 and 226; (52) SEQ ID NOS: 140 and 217; (53) SEQ ID NOS: 172 and 247; (54) SEQ ID NOS: 143 and 230; (55) SEQ ID NOS: 139 and 224; (56) SEQ ID NOS: 150 and 235; (57) SEQ ID NOS: 184 and 253; (58) SEQ ID NOS: 168 and 255; (59) SEQ ID NOS: 175 and 255; (60) SEQ ID NOS: 173 and 253; (61) SEQ ID NOS: 164 and 255; (62) SEQ ID NOS: 177 and 253; (63) SEQ ID NOS: 169 and 253; (64) SEQ ID NOS: 183 and 255; (65) SEQ ID NOS: 172 and 245; (66) SEQ ID NOS: 166 and 254; (67) SEQ ID NOS: 178 and 254; (68) SEQ ID NOS: 165 and 255; (69) SEQ ID NOS: 174 and 253; (70) SEQ ID NOS: 171 and 254; (71) SEQ ID NOS: 160 and 255; (72) SEQ ID NOS: 172 and 246; (73) SEQ ID NOS: 175 and 254; (74) SEQ ID NOS: 161 and 253; (75) SEQ ID NOS: 159 and 255; (76) SEQ ID NOS: 167 and 253; (77) SEQ ID NOS: 183 and 254; (78) SEQ ID NOS: 181 and 255; (79) SEQ ID NOS: 150 and 240; (80) SEQ ID NOS: 179 and 253; (81) SEQ ID NOS: 172 and 254; (82) SEQ ID NOS: 176 and 253; (83) SEQ ID NOS: 183 and 253; (84) SEQ ID NOS: 166 and 255; (85) SEQ ID NOS: 154 and 239; (86) SEQ ID NOS: 178 and 253; (87) SEQ ID NOS: 153 and 239; (88) SEQ ID NOS: 152 and 239; (89) SEQ ID NOS: 185 and 254; (90) SEQ ID NOS: 171 and 255; (91) SEQ ID NOS: 155 and 239; (92) SEQ ID NOS: 182 and 255; (93) SEQ ID NOS:180 and 254; (94) SEQ ID NOS: 172 and 244, and (95) SEQ ID NOS: 183 and 253.

13. The method of claim 10, wherein the forward and reverse primer pair are selected from the group consisting of: (1) SEQ ID NOS: 256 and 292; (2) SEQ ID NOS: 257 and 292; (3) SEQ ID NOS: 257 and 293; (4) SEQ ID NOS: 258 and 292; (5) SEQ ID NOS: 259 and 292; (6) SEQ ID NOS: 260 and 292; (7) SEQ ID NOS: 261 and 292; (8) SEQ ID NOS: 262 and 292; (9) SEQ ID NOS: 263 and 293; (10) SEQ ID NOS: 264 and 294; ( 1) SEQ ID NOS: 264 and 295 (12) SEQ ID NOS: 264 and 296; (13) SEQ ID NOS: 264 and 297; (14) SEQ ID NOS: 264 and 298; (15) SEQ ID NOS: 264 and 299; (16) SEQ ID NOS: 264 and 300; (17) SEQ ID NOS: 265 and 294; (18) SEQ ID NOS: 265 and 301; (19) SEQ ID NOS: 265 and 302; (20) SEQ ID NOS: 265 and 303; (21) SEQ ID NOS: 265 and 304, (22) SEQ ID NOS: 265 and 305; (23) SEQ ID NOS: 265 and 299; (24) SEQ ID NOS: 265 and 306; (25) SEQ ID NOS: 265 and 307; (26) SEQ ID NOS: 266 and 294, (27) SEQ ID NOS: 266 and 301; (28) SEQ ID NOS: 266 and 296; (29) SEQ ID NOS: 266 and 303; (30) SEQ ID NOS: 266 and 298; (31) SEQ ID NOS: 266 and 308; (32) SEQ ID NOS: 266 and 309; (33) SEQ ID NOS: 266 and 300; (34) SEQ ID NOS: 267 and 294; (35) SEQ ID NOS: 267 and 301; (36) SEQ ID NOS: 267 and 296; (37) SEQ ID NOS: 267 and 303; (38) SEQ ID NOS: 267 and 298; (39) SEQ ID NOS: 267 and 305; (40) SEQ ID NOS: 267 and 299; (41) SEQ ID NOS: 267 and 310; (42) SEQ ID NOS: 267 and 300; (43) SEQ ID NOS: 268 and 308; (44) SEQ ID NOS: 268 and 299; (45) SEQ ID NOS: 268 and 310; (46) SEQ ID NOS: 268 and 300; (47) SEQ ID NOS: 269 and 311; (48) SEQ ID NOS: 269 and 312; (49) SEQ ID NOS: 269 and 313; (50) SEQ ID NOS: 269 and 314; (51) SEQ ID NOS: 269 and 315; (52) SEQ ID NOS: 269 and 316; (53) SEQ ID NOS: 269 and 306; (54) SEQ ID NOS: 270 and 294; (55) SEQ ID NOS: 270 and 317; (56) SEQ ID NOS:270 and 303; (57) SEQ ID NOS: 270 and 298; (58) SEQ ID NOS: 270 and 308; (59) SEQ ID NOS: 270 and 309; (60) SEQ ID NOS: 270 and 310; (61) SEQ ID NOS: 270 and 300; (62) SEQ ID NOS:271 and 294; (63) SEQ ID NOS: 271 and 301; (64) SEQ ID NOS: 271 and 296; (65) SEQ ID NOS: 271 and 303; (66) SEQ ID NOS: 271 and 298; (67) SEQ ID NOS: 271 and 305; (68) SEQ ID NOS: 271 and 318; (69) SEQ ID NOS: 271 and 309; (70) SEQ ID NOS: 271 and 319; (71) SEQ ID NOS: 271 and 300; (72) SEQ ID NOS: 272 and 320; (73) SEQ ID NOS: 272 and 310; (74) SEQ ID NOS: 272 and 307; (75) SEQ ID NOS: 272 and 321; (76) SEQ ID NOS: 272 and 299; (77) SEQ ID NOS: 273 and 322; (78) SEQ ID NOS: 273 and 323; (79) SEQ ID NOS: 273 and 324; (80) SEQ ID NOS: 274 and 325; (81) SEQ ID NOS: 274 and 326; (82) SEQ ID NOS: 274 and 327; (83) SEQ ID NOS: 274 and 328; (84) SEQ ID NOS: 275 and 325; (85) SEQ ID NOS: 275 and 323; (86) SEQ ID NOS: 275 and 327; (87) SEQ ID NOS: 276 and 323; (88) SEQ ID NOS: 276 and 326; (89) SEQ ID NOS: 277 and 325; (90) SEQ ID NOS: 277 and 326; (91) SEQ ID NOS: 277 and 327; (92) SEQ ID NOS: 278 and 325; (93) SEQ ID NOS: 278 and 323; (94) SEQ ID NOS: 278 and 326; (95) SEQ ID NOS: 278 and 327; (96) SEQ ID NOS: 279 and 325; (97) SEQ ID NOS: 279 and 326; (98) SEQ ID NOS: 279 and 327, (99) SEQ ID NOS: 279 and 328; (1 00) SEQ ID NOS: 280 and 325; (101) SEQ ID NOS: 280 and 326; (102) SEQ ID NOS: 280 and 327; (103) SEQ ID NOS: 280 and 328; (104) SEQ ID NOS: 281 and 325; (105) SEQ ID NOS: 281 and 323; (106) SEQ ID NOS: 281 and 327; (107) SEQ ID NOS: 281 and 328; (108) SEQ ID NOS: 329 and 331; (109) SEQ ID NOS: 330 and 321; and (110) SEQ ID NOS: 329 and 331.

14. The method of claim 10, wherein step (3) is performed using one or more labeled probes comprising a sequence that hybridizes to the amplicon generated by the forward and reverse primer pair group of Tables 2, 3 or 4.

15. The method of claim 14, wherein the sequence of the one or more labeled probes is selected from the group consisting of: SEQ ID NOS: 2, 4, 5, 11, 14, 16, 18, 24, 27, 38, 40, 41, 53, 56, 59, 61, 63, 64, 65, 69, 71, 77, 81, 83, 85, 86, 93, 96, 99, 101, 102, 108, 110, 122, 124, 127, 133-134, 186-214, 282-291, and 332-333.

16. The method of claim 14, wherein the one or more labeled probes is fluorescently labeled and the step of detecting the binding of the one or more labeled probes to the amplified PCR product comprises measuring the fluorescence of the sample.

17. The method of claim 14, wherein the one or more labeled probes is fluorescently labeled and the step of quantitating the binding of the one or more labeled probes to the amplified PCR product comprises measuring the fluorescence of the sample.

18. The method of claim 14, wherein the sample is selected from the group consisting of: blood, serum, plasma, sputum, urine, stool, skin, cerebrospinal fluid, saliva, semen, seminal fluid, gastric secretions, tears, oropharyngeal swabs, nasopharyngeal swabs, throat swabs, nasal aspirates, nasal wash, renal tissue and fluid therefrom including perfusion media, fluids collected from the ear, eye, mouth, and respiratory airways, neoplastic tissue obtained from biopsies, tissues utilized for transplantation between individuals and/or animal derived tissues utilized for transplantation into a human recipient, fluids and cells derived from the culturing of human cells, human stem cells, human cartilage and fibroblasts.

19. The method of claim 14, wherein the sample is a human or a mammalian sample to be utilized in or on a human subject.

20. A polynucleotide comprising a nucleotide sequence selected from the group consisting of: SEQ ID NOS: 1-334, for isolating nucleic acid sequences derived from any BK virus genome.

21. A polynucleotide comprising a nucleotide sequence selected from the group consisting of: SEQ ID NOS: 1-3347 for sequencing nucleic acid sequences derived from any BK virus genome.

22. A polynucleotide probe comprising SEQ ID NO:135 for binding to an internal control target sequence comprising SEQ ID NO:335.

23. A polynucleotide probe comprising SEQ ID NO:334 for binding to an internal control target sequence comprising SEQ ID NO:336.

24. The probe of claim 22 or 23, wherein the probe monitors the efficiency of extraction of nucleic acids from a sample, and the amplification step of PCR.

25. A method for detecting a BKV-associated condition or disease, comprising:
(1) contacting at least one forward and reverse primer pair listed in Tables 2, 3 or 4 to a sample;
(2) conducting a polymerase chain reaction; and
(3) detecting and/or quantitating the generation of an amplified PCR product,
wherein the generation of an amplified PCR product indicates the presence of BK virus in the sample.

26. The method of claim 25, wherein the BKV-associated condition or disease is a BKV-associated neuropathy or BKV infection in a sample selected from the group consisting of: renal epithelial cells, brain tissue. neoplastic tissue obtained from biopsies, cerebrospinal fluid, and tissues utilized for transplantation.


SEQUENCE LISTING (340 sequences)

SEQ ID NO: 1 - DNA, 20 bp, Artificial Sequence
  Description of Artificial Sequence Synthetic primer
acagcccagtcccaggtaat

SEQ ID NO: 2 - DNA, 28 bp, Artificial Sequence
  Description of Artificial Sequence Synthetic probe
atacataggctgcccatccactctctgg

SEQ ID NO: 3 - DNA, 22 bp, Artificial Sequence
  Description of Artificial Sequence Synthetic primer
agtctttctgtgccatcaaaca

SEQ ID NO: 4 - DNA, 31 bp, Artificial Sequence
  Description of Artificial Sequence Synthetic probe
ataccatacataggctgcccatccactctct

SEQ ID NO: 5 - DNA, 30 bp, Artificial Sequence
  Description of Artificial Sequence Synthetic probe
cataccatacataggctgcccatccactct

SEQ ID NO: 6 - DNA, 22 bp, Artificial Sequence
  Description of Artificial Sequence Synthetic primer
ggcctatttggacaaaaacaat

SEQ ID NO: 7 - DNA, 22 bp, Artificial Sequence
  Description of Artificial Sequence Synthetic primer
gcctatttggacaaaaacaatg

SEQ ID NO: 8 - DNA, 22 bp, Artificial Sequence
  Description of Artificial Sequence Synthetic primer
cctatttggacaaaaacaatgc

SEQ ID NO: 9 - DNA, 22 bp, Artificial Sequence
  Description of Artificial Sequence Synthetic primer
aagattcgcctgagaaaaagat

SEQ ID NO: 10 - DNA, 19 bp, Artificial Sequence
  Description of Artificial Sequence Synthetic primer
aggggcagctcccaaaaag

SEQ ID NO: 11 - DNA, 37 bp, Artificial Sequence
  Description of Artificial Sequence Synthetic probe
caaaggaacccgtgcaagtgccaaaactactaataaa

SEQ ID NO: 12 - DNA, 24 bp, Artificial Sequence
  Description of Artificial Sequence Synthetic primer
gatttccacaggttaggtcctcat

SEQ ID NO: 13 - DNA, 17 bp, Artificial Sequence
  Description of Artificial Sequence Synthetic primer
caggggcagctcccaaa

SEQ ID NO: 14 - DNA, 36 bp, Artificial Sequence
  Description of Artificial Sequence Synthetic probe
aaaggaacccgtgcaagtgccaaaactactaataaa

SEQ ID NO: 15 - DNA, 18 bp, Artificial Sequence
  Description of Artificial Sequence Synthetic primer
gggcagctcccaaaaagc

SEQ ID NO: 16 - DNA, 33 bp, Artificial Sequence
  Description of Artificial Sequence Synthetic probe
aaaggaacccgtgcaagtgccaaaactactaat

SEQ ID NO: 17 - DNA, 26 bp, Artificial Sequence
  Description of Artificial Sequence Synthetic primer
tagatttccacaggttaggtcctcat

SEQ ID NO: 18 - DNA, 35 bp, Artificial Sequence
  Description of Artificial Sequence Synthetic probe
aaggaacccgtgcaagtgccaaaactactaataaa

SEQ ID NO: 19 - DNA, 27 bp, Artificial Sequence
  Description of Artificial Sequence Synthetic primer
gtagatttccacaggttaggtcctcat

SEQ ID NO: 20 - DNA, 18 bp, Artificial Sequence
  Description of Artificial Sequence Synthetic primer
caggggcagctcccaaaa

SEQ ID NO: 21 - DNA, 25 bp, Artificial Sequence
  Description of Artificial Sequence Synthetic primer
agatttccacaggttaggtcctcat

SEQ ID NO: 22 - DNA, 28 bp, Artificial Sequence
  Description of Artificial Sequence Synthetic primer
agtagatttccacaggttaggtcctcat

SEQ ID NO: 23 - DNA, 17 bp, Artificial Sequence
  Description of Artificial Sequence Synthetic primer
aggggcagctcccaaaa

SEQ ID NO: 24 - DNA, 32 bp, Artificial Sequence
  Description of Artificial Sequence Synthetic probe
aaggaacccgtgcaagtgccaaaactactaat

SEQ ID NO: 25 - DNA, 25 bp, Artificial Sequence
  Description of Artificial Sequence Synthetic primer
gcatctaccccagttttaacttcta

SEQ ID NO: 26 - DNA, 22 bp, Artificial Sequence
  Description of Artificial Sequence Synthetic primer
ccaaccaaaagaaaaggagagt

SEQ ID NO: 27 - DNA, 25 bp, Artificial Sequence
  Description of Artificial Sequence Synthetic probe
cagctcccaaaaagccaaaggaacc

SEQ ID NO: 28 - DNA, 18 bp, Artificial Sequence
  Description of Artificial Sequence Synthetic primer
ggggcagctcccaaaaag

SEQ ID NO: 29 - DNA, 20 bp, Artificial Sequence
  Description of Artificial Sequence Synthetic primer
atctggatcccccatttctg

SEQ ID NO: 30 - DNA, 21 bp, Artificial Sequence
  Description of Artificial Sequence Synthetic primer
catctggatcccccatttctg

SEQ ID NO: 31 - DNA, 21 bp, Artificial Sequence
  Description of Artificial Sequence Synthetic primer
tcatctggatcccccatttct

SEQ ID NO: 32 - DNA, 27 bp, Artificial Sequence
  Description of Artificial Sequence Synthetic primer
tagatttccacaggttaggtcctcatt

SEQ ID NO: 33 - DNA, 28 bp, Artificial Sequence
  Description of Artificial Sequence Synthetic primer
gtagatttccacaggttaggtcctcatt

SEQ ID NO: 34 - DNA, 29 bp, Artificial Sequence
  Description of Artificial Sequence Synthetic primer
agtagatttccacaggttaggtcctcatt

SEQ ID NO: 35 - DNA, 21 bp, Artificial Sequence
  Description of Artificial Sequence Synthetic primer
gggaattcttgctgtgctgta

SEQ ID NO: 36 - DNA, 22 bp, Artificial Sequence
  Description of Artificial Sequence Synthetic primer
ttcatctggatcccccatttct

SEQ ID NO: 37 - DNA, 24 bp, Artificial Sequence
  Description of Artificial Sequence Synthetic primer
ccaaccaaaagaaaaggagagtgt

SEQ ID NO: 38 - DNA, 26 bp, Artificial Sequence
  Description of Artificial Sequence Synthetic probe
cagctcccaaaaagccaaaggaaccc

SEQ ID NO: 39 - DNA, 20 bp, Artificial Sequence
  Description of Artificial Sequence Synthetic primer
catctggatcccccatttct

SEQ ID NO: 40 - DNA, 37 bp, Artificial Sequence
  Description of Artificial Sequence Synthetic probe
aaggaacccgtgcaagtgccaaaactactaataaaag

SEQ ID NO: 41 - DNA, 38 bp, Artificial Sequence
  Description of Artificial Sequence Synthetic probe
aaaggaacccgtgcaagtgccaaaactactaataaaag

SEQ ID NO: 42 - DNA, 18 bp, Artificial Sequence
  Description of Artificial Sequence Synthetic primer
aggggcagctcccaaaaa

SEQ ID NO: 43 - DNA, 22 bp, Artificial Sequence
  Description of Artificial Sequence Synthetic primer
gggaattcttgctgtgctgtaa

SEQ ID NO: 44 - DNA, 22 bp, Artificial Sequence
  Description of Artificial Sequence Synthetic primer
ggggaattcttgctgtgctgta

SEQ ID NO: 45 - DNA, 23 bp, Artificial Sequence
  Description of Artificial Sequence Synthetic primer
gggaattcttgctgtgctgtaac

SEQ ID NO: 46 - DNA, 23 bp, Artificial Sequence
  Description of Artificial Sequence Synthetic primer
aggggaattcttgctgtgctgta

SEQ ID NO: 47 - DNA, 23 bp, Artificial Sequence
  Description of Artificial Sequence Synthetic primer
ggggaattcttgctgtgctgtaa

SEQ ID NO: 48 - DNA, 24 bp, Artificial Sequence
  Description of Artificial Sequence Synthetic primer
ggggaattcttgctgtgctgtaac

SEQ ID NO: 49 - DNA, 24 bp, Artificial Sequence
  Description of Artificial Sequence Synthetic primer
aggggaattcttgctgtgctgtaa

SEQ ID NO: 50 - DNA, 22 bp, Artificial Sequence
  Description of Artificial Sequence Synthetic primer
tttcatctggatcccccatttc

SEQ ID NO: 51 - DNA, 23 bp, Artificial Sequence
  Description of Artificial Sequence Synthetic primer
tttcatctggatcccccatttct

SEQ ID NO: 52 - DNA, 16 bp, Artificial Sequence
  Description of Artificial Sequence Synthetic primer
ggggcagctcccaaaa

SEQ ID NO: 53 - DNA, 25 bp, Artificial Sequence
  Description of Artificial Sequence Synthetic probe
aaggaacccgtgcaagtgccaaaac

SEQ ID NO: 54 - DNA, 18 bp, Artificial Sequence
  Description of Artificial Sequence Synthetic primer
gggaggggaattcttgct

SEQ ID NO: 55 - DNA, 23 bp, Artificial Sequence
  Description of Artificial Sequence Synthetic primer
cccaaccaaaagaaaaggagagt

SEQ ID NO: 56 - DNA, 26 bp, Artificial Sequence
  Description of Artificial Sequence Synthetic probe
caaaggaacccgtgcaagtgccaaaa

SEQ ID NO: 57 - DNA, 23 bp, Artificial Sequence
  Description of Artificial Sequence Synthetic primer
atttccacaggttaggtcctcat

SEQ ID NO: 58 - DNA, 24 bp, Artificial Sequence
  Description of Artificial Sequence Synthetic primer
ccccaaccaaaagaaaaggagagt

SEQ ID NO: 59 - DNA, 32 bp, Artificial Sequence
  Description of Artificial Sequence Synthetic probe
caaaggaacccgtgcaagtgccaaaactacta

SEQ ID NO: 60 - DNA, 25 bp, Artificial Sequence
  Description of Artificial Sequence Synthetic primer
cccaaccaaaagaaaaggagagtgt

SEQ ID NO: 61 - DNA, 34 bp, Artificial Sequence
  Description of Artificial Sequence Synthetic probe
caaaggaacccgtgcaagtgccaaaactactaat

SEQ ID NO: 62 - DNA, 26 bp, Artificial Sequence
  Description of Artificial Sequence Synthetic primer
agatttccacaggttaggtcctcatt

SEQ ID NO: 63 - DNA, 25 bp, Artificial Sequence
  Description of Artificial Sequence Synthetic probe
tcccaaaaagccaaaggaacccgtg

SEQ ID NO: 64 - DNA, 26 bp, Artificial Sequence
  Description of Artificial Sequence Synthetic probe
agctcccaaaaagccaaaggaacccg

SEQ ID NO: 65 - DNA, 27 bp, Artificial Sequence
  Description of Artificial Sequence Synthetic probe
agctcccaaaaagccaaaggaacccgt

SEQ ID NO: 66 - DNA, 24 bp, Artificial Sequence
  Description of Artificial Sequence Synthetic primer
tctgttatagcatctaccccagtt

SEQ ID NO: 67 - DNA, 27 bp, Artificial Sequence
  Description of Artificial Sequence Synthetic primer
agttacagcctcccacatcagtagatt

SEQ ID NO: 68 - DNA, 19 bp, Artificial Sequence
  Description of Artificial Sequence Synthetic primer
caggggcagctcccaaaaa

SEQ ID NO: 69 - DNA, 28 bp, Artificial Sequence
  Description of Artificial Sequence Synthetic probe
caaaggaacccgtgcaagtgccaaaact

SEQ ID NO: 70 - DNA, 22 bp, Artificial Sequence
  Description of Artificial Sequence Synthetic primer
tcatctggatcccccatttctg

SEQ ID NO: 71 - DNA, 36 bp, Artificial Sequence
  Description of Artificial Sequence Synthetic probe
aaggaacccgtgcaagtgccaaaactactaataaaa

SEQ ID NO: 72 - DNA, 26 bp, Artificial Sequence
  Description of Artificial Sequence Synthetic primer
gttacagcctcccacatcagtagatt

SEQ ID NO: 73 - DNA, 27 bp, Artificial Sequence
  Description of Artificial Sequence Synthetic primer
cctctgttatagcatctaccccagttt

SEQ ID NO: 74 - DNA, 27 bp, Artificial Sequence
  Description of Artificial Sequence Synthetic primer
acctctgttatagcatctaccccagtt

SEQ ID NO: 75 - DNA, 28 bp, Artificial Sequence
  Description of Artificial Sequence Synthetic primer
acctctgttatagcatctaccccagttt

SEQ ID NO: 76 - DNA, 17 bp, Artificial Sequence
  Description of Artificial Sequence Synthetic primer
ggcagctcccaaaaagc

SEQ ID NO: 77 - DNA, 33 bp, Artificial Sequence
  Description of Artificial Sequence Synthetic probe
ctccttttattagtagttttggcacttgcacgg

SEQ ID NO: 78 - DNA, 27 bp, Artificial Sequence
  Description of Artificial Sequence Synthetic primer
tccttttattagtagttttggcacttg

SEQ ID NO: 79 - DNA, 26 bp, Artificial Sequence
  Description of Artificial Sequence Synthetic primer
ccttttattagtagttttggcacttg

SEQ ID NO: 80 - DNA, 23 bp, Artificial Sequence
  Description of Artificial Sequence Synthetic primer
caaccaaaagaaaaggagagtgt

SEQ ID NO: 81 - DNA, 23 bp, Artificial Sequence
  Description of Artificial Sequence Synthetic probe
aaggaacccgtgcaagtgccaaa

SEQ ID NO: 82 - DNA, 19 bp, Artificial Sequence
  Description of Artificial Sequence Synthetic primer
tcatctggatcccccattt

SEQ ID NO: 83 - DNA, 35 bp, Artificial Sequence
  Description of Artificial Sequence Synthetic probe
aaaggaacccgtgcaagtgccaaaactactaataa

SEQ ID NO: 84 - DNA, 30 bp, Artificial Sequence
  Description of Artificial Sequence Synthetic primer
tacctctgttatagcatctaccccagtttt

SEQ ID NO: 85 - DNA, 33 bp, Artificial Sequence
  Description of Artificial Sequence Synthetic probe
cccgtgcaagtgccaaaactactaataaaagga

SEQ ID NO: 86 - DNA, 30 bp, Artificial Sequence
  Description of Artificial Sequence Synthetic probe
taaggttttcatctggatcccccatttctg

SEQ ID NO: 87 - DNA, 19 bp, Artificial Sequence
  Description of Artificial Sequence Synthetic primer
ctcccaaaaagccaaagga

SEQ ID NO: 88 - DNA, 19 bp, Artificial Sequence
  Description of Artificial Sequence Synthetic primer
agctcccaaaaagccaaag

SEQ ID NO: 89 - DNA, 23 bp, Artificial Sequence
  Description of Artificial Sequence Synthetic primer
ttttcatctggatcccccatttc

SEQ ID NO: 90 - DNA, 26 bp, Artificial Sequence
  Description of Artificial Sequence Synthetic primer
actaaagcccctaaggttttcatctg

SEQ ID NO: 91 - DNA, 27 bp, Artificial Sequence
  Description of Artificial Sequence Synthetic primer
gactaaagcccctaaggttttcatctg

SEQ ID NO: 92 - DNA, 24 bp, Artificial Sequence
  Description of Artificial Sequence Synthetic primer
atgaaaaccttaggggctttagtc

SEQ ID NO: 93 - DNA, 27 bp, Artificial Sequence
  Description of Artificial Sequence Synthetic probe
ccctgttacagcacagcaagaattccc

SEQ ID NO: 94 - DNA, 22 bp, Artificial Sequence
  Description of Artificial Sequence Synthetic primer
tttccacaggttaggtcctcat

SEQ ID NO: 95 - DNA, 23 bp, Artificial Sequence
  Description of Artificial Sequence Synthetic primer
tgaaaaccttaggggctttagtc

SEQ ID NO: 96 - DNA, 28 bp, Artificial Sequence
  Description of Artificial Sequence Synthetic probe
cctgttacagcacagcaagaattcccct

SEQ ID NO: 97 - DNA, 25 bp, Artificial Sequence
  Description of Artificial Sequence Synthetic primer
atgaaaaccttaggggctttagtct

SEQ ID NO: 98 - DNA, 23 bp, Artificial Sequence
  Description of Artificial Sequence Synthetic primer
cagtgatagcccagacagaaaaa

SEQ ID NO: 99 - DNA, 29 bp, Artificial Sequence
  Description of Artificial Sequence Synthetic probe
ccctgttacagcacagcaagaattcccct

SEQ ID NO: 100 - DNA, 23 bp, Artificial Sequence
  Description of Artificial Sequence Synthetic primer
gaaaaccttaggggctttagtct

SEQ ID NO: 101 - DNA, 27 bp, Artificial Sequence
  Description of Artificial Sequence Synthetic probe
cctgttacagcacagcaagaattcccc

SEQ ID NO: 102 - DNA, 28 bp, Artificial Sequence
  Description of Artificial Sequence Synthetic probe
tccctgttacagcacagcaagaattccc

SEQ ID NO: 103 - DNA, 23 bp, Artificial Sequence
  Description of Artificial Sequence Synthetic primer
tttccacaggttaggtcctcatt

SEQ ID NO: 104 - DNA, 24 bp, Artificial Sequence
  Description of Artificial Sequence Synthetic primer
gatgaaaaccttaggggctttagt

SEQ ID NO: 105 - DNA, 24 bp, Artificial Sequence
  Description of Artificial Sequence Synthetic primer
atttccacaggttaggtcctcatt

SEQ ID NO: 106 - DNA, 25 bp, Artificial Sequence
  Description of Artificial Sequence Synthetic primer
agatgaaaaccttaggggctttagt

SEQ ID NO: 107 - DNA, 26 bp, Artificial Sequence
  Description of Artificial Sequence Synthetic primer
gatgaaaaccttaggggctttagtct

SEQ ID NO: 108 - DNA, 31 bp, Artificial Sequence
  Description of Artificial Sequence Synthetic probe
cttccctgttacagcacagcaagaattcccc

SEQ ID NO: 109 - DNA, 25 bp, Artificial Sequence
  Description of Artificial Sequence Synthetic primer
cagatgaaaaccttaggggctttag

SEQ ID NO: 110 - DNA, 31 bp, Artificial Sequence
  Description of Artificial Sequence Synthetic probe
ttccctgttacagcacagcaagaattcccct

SEQ ID NO: 111 - DNA, 24 bp, Artificial Sequence
  Description of Artificial Sequence Synthetic primer
tgaaaaccttaggggctttagtct

SEQ ID NO: 112 - DNA, 25 bp, Artificial Sequence
  Description of Artificial Sequence Synthetic primer
gatttccacaggttaggtcctcatt

SEQ ID NO: 113 - DNA, 20 bp, Artificial Sequence
  Description of Artificial Sequence Synthetic primer
agaaatgggggatccagatg

SEQ ID NO: 114 - DNA, 21 bp, Artificial Sequence
  Description of Artificial Sequence Synthetic primer
atgggggatccagatgaaaac

SEQ ID NO: 115 - DNA, 20 bp, Artificial Sequence
  Description of Artificial Sequence Synthetic primer
gaaatgggggatccagatga

SEQ ID NO: 116 - DNA, 22 bp, Artificial Sequence
  Description of Artificial Sequence Synthetic primer
ttccacaggttaggtcctcatt

SEQ ID NO: 117 - DNA, 20 bp, Artificial Sequence
  Description of Artificial Sequence Synthetic primer
aaatgggggatccagatgaa

SEQ ID NO: 118 - DNA, 20 bp, Artificial Sequence
  Description of Artificial Sequence Synthetic primer
aatgggggatccagatgaaa

SEQ ID NO: 119 - DNA, 20 bp, Artificial Sequence
  Description of Artificial Sequence Synthetic primer
tgggggatccagatgaaaac

SEQ ID NO: 120 - DNA, 21 bp, Artificial Sequence
  Description of Artificial Sequence Synthetic primer
gaaatgggggatccagatgaa

SEQ ID NO: 121 - DNA, 21 bp, Artificial Sequence
  Description of Artificial Sequence Synthetic primer
aaatgggggatccagatgaaa

SEQ ID NO: 122 - DNA, 30 bp, Artificial Sequence
  Description of Artificial Sequence Synthetic probe
tccctgttacagcacagcaagaattcccct

SEQ ID NO: 123 - DNA, 21 bp, Artificial Sequence
  Description of Artificial Sequence Synthetic primer
agaaatgggggatccagatga

SEQ ID NO: 124 - DNA, 30 bp, Artificial Sequence
  Description of Artificial Sequence Synthetic probe
ttccctgttacagcacagcaagaattcccc

SEQ ID NO: 125 - DNA, 21 bp, Artificial Sequence
  Description of Artificial Sequence Synthetic primer
aatgggggatccagatgaaaa

SEQ ID NO: 126 - DNA, 21 bp, Artificial Sequence
  Description of Artificial Sequence Synthetic primer
cagaaatgggggatccagatg

SEQ ID NO: 127 - DNA, 32 bp, Artificial Sequence
  Description of Artificial Sequence Synthetic probe
cttccctgttacagcacagcaagaattcccct

SEQ ID NO: 128 - DNA, 26 bp, Artificial Sequence
  Description of Artificial Sequence Synthetic primer
cagatgaaaaccttaggggctttagt

SEQ ID NO: 129 - DNA, 22 bp, Artificial Sequence
  Description of Artificial Sequence Synthetic primer
aatgggggatccagatgaaaac

SEQ ID NO: 130 - DNA, 22 bp, Artificial Sequence
  Description of Artificial Sequence Synthetic primer
agaaatgggggatccagatgaa

SEQ ID NO: 131 - DNA, 21 bp, Artificial Sequence
  Description of Artificial Sequence Synthetic primer
gcttcctaaacccagaaatgg

SEQ ID NO: 132 - DNA, 22 bp, Artificial Sequence
  Description of Artificial Sequence Synthetic primer
atgcttcctaaacccagaaatg

SEQ ID NO: 133 - DNA, 28 bp, Artificial Sequence
  Description of Artificial Sequence Synthetic probe
tgttacagcacagcaagaattcccctcc

SEQ ID NO: 134 - DNA, 30 bp, Artificial Sequence
  Description of Artificial Sequence Synthetic probe
cttccctgttacagcacagcaagaattccc

SEQ ID NO: 135 - DNA, 29 bp, Artificial Sequence
  Description of Artificial Sequence Synthetic probe
ccagccttgatcgttacaaaggacgatgc

SEQ ID NO: 136 - DNA, 18 bp, Artificial Sequence
  Description of Artificial Sequence Synthetic primer
taaaagactcctaggtaa

SEQ ID NO: 137 - DNA, 19 bp, Artificial Sequence
  Description of Artificial Sequence Synthetic primer
taaaagactcctaggtaag

SEQ ID NO: 138 - DNA, 21 bp, Artificial Sequence
  Description of Artificial Sequence Synthetic primer
taaaagactcctaggtaagta

SEQ ID NO: 139 - DNA, 22 bp, Artificial Sequence
  Description of Artificial Sequence Synthetic primer
taaaagactcctaggtaagtaa

SEQ ID NO: 140 - DNA, 20 bp, Artificial Sequence
  Description of Artificial Sequence Synthetic primer
aaaagactcctaggtaagta

SEQ ID NO: 141 - DNA, 22 bp, Artificial Sequence
  Description of Artificial Sequence Synthetic primer
aaaagactcctaggtaagtaat

SEQ ID NO: 142 - DNA, 20 bp, Artificial Sequence
  Description of Artificial Sequence Synthetic primer
aaagactcctaggtaagtaa

SEQ ID NO: 143 - DNA, 21 bp, Artificial Sequence
  Description of Artificial Sequence Synthetic primer
aaagactcctaggtaagtaat

SEQ ID NO: 144 - DNA, 18 bp, Artificial Sequence
  Description of Artificial Sequence Synthetic primer
aagactcctaggtaagta

SEQ ID NO: 145 - DNA, 19 bp, Artificial Sequence
  Description of Artificial Sequence Synthetic primer
aagactcctaggtaagtaa

SEQ ID NO: 146 - DNA, 20 bp, Artificial Sequence
  Description of Artificial Sequence Synthetic primer
aagactcctaggtaagtaat

SEQ ID NO: 147 - DNA, 19 bp, Artificial Sequence
  Description of Artificial Sequence Synthetic primer
agactcctaggtaagtaat

SEQ ID NO: 148 - DNA, 18 bp, Artificial Sequence
  Description of Artificial Sequence Synthetic primer
gactcctaggtaagtaat

SEQ ID NO: 149 - DNA, 17 bp, Artificial Sequence
  Description of Artificial Sequence Synthetic primer
gtgctgctctagcactt

SEQ ID NO: 150 - DNA, 18 bp, Artificial Sequence
  Description of Artificial Sequence Synthetic primer
gtgctgctctagcacttt

SEQ ID NO: 151 - DNA, 18 bp, Artificial Sequence
  Description of Artificial Sequence Synthetic primer
ctgctctagcacttttgg

SEQ ID NO: 152 - DNA, 15 bp, Artificial Sequence
  Description of Artificial Sequence Synthetic primer
cttttgggggaccta

SEQ ID NO: 153 - DNA, 15 bp, Artificial Sequence
  Description of Artificial Sequence Synthetic primer
tgggggacctagttg

SEQ ID NO: 154 - DNA, 16 bp, Artificial Sequence
  Description of Artificial Sequence Synthetic primer
cctagttgccagtgta

SEQ ID NO: 155 - DNA, 16 bp, Artificial Sequence
  Description of Artificial Sequence Synthetic primer
agttgccagtgtatct

SEQ ID NO: 156 - DNA, 16 bp, Artificial Sequence
  Description of Artificial Sequence Synthetic primer
ttgccagtgtatctga

SEQ ID NO: 157 - DNA, 19 bp, Artificial Sequence
  Description of Artificial Sequence Synthetic primer
caaattgcatcccttgcta

SEQ ID NO: 158 - DNA, 17 bp, Artificial Sequence
  Description of Artificial Sequence Synthetic primer
aaattgcatcccttgct

SEQ ID NO: 159 - DNA, 16 bp, Artificial Sequence
  Description of Artificial Sequence Synthetic primer
aattgcatcccttgct

SEQ ID NO: 160 - DNA, 17 bp, Artificial Sequence
  Description of Artificial Sequence Synthetic primer
aattgcatcccttgcta

SEQ ID NO: 161 - DNA, 15 bp, Artificial Sequence
  Description of Artificial Sequence Synthetic primer
attgcatcccttgct

SEQ ID NO: 162 - DNA, 18 bp, Artificial Sequence
  Description of Artificial Sequence Synthetic primer
attgcatcccttgctact

SEQ ID NO: 163 - DNA, 18 bp, Artificial Sequence
  Description of Artificial Sequence Synthetic primer
ttgcatcccttgctactg

SEQ ID NO: 164 - DNA, 16 bp, Artificial Sequence
  Description of Artificial Sequence Synthetic primer
tgcatcccttgctact

SEQ ID NO: 165 - DNA, 17 bp, Artificial Sequence
  Description of Artificial Sequence Synthetic primer
tgcatcccttgctactg

SEQ ID NO: 166 - DNA, 16 bp, Artificial Sequence
  Description of Artificial Sequence Synthetic primer
gcatcccttgctactg

SEQ ID NO: 167 - DNA, 19 bp, Artificial Sequence
  Description of Artificial Sequence Synthetic primer
ctcaaacatatgctgtaat

SEQ ID NO: 168 - DNA, 20 bp, Artificial Sequence
  Description of Artificial Sequence Synthetic primer
tcaaacatatgctgtaattg

SEQ ID NO: 169 - DNA, 19 bp, Artificial Sequence
  Description of Artificial Sequence Synthetic primer
caaacatatgctgtaattg

SEQ ID NO: 170 - DNA, 19 bp, Artificial Sequence
  Description of Artificial Sequence Synthetic primer
aacatatgctgtaattgct

SEQ ID NO: 171 - DNA, 18 bp, Artificial Sequence
  Description of Artificial Sequence Synthetic primer
acatatgctgtaattgct

SEQ ID NO: 172 - DNA, 17 bp, Artificial Sequence
  Description of Artificial Sequence Synthetic primer
catatgctgtaattgct

SEQ ID NO: 173 - DNA, 16 bp, Artificial Sequence
  Description of Artificial Sequence Synthetic primer
ggctattgctgggttt

SEQ ID NO: 174 - DNA, 15 bp, Artificial Sequence
  Description of Artificial Sequence Synthetic primer
gctattgctgggttt

SEQ ID NO: 175 - DNA, 16 bp, Artificial Sequence
  Description of Artificial Sequence Synthetic primer
gctattgctgggtttg

SEQ ID NO: 176 - DNA, 16 bp, Artificial Sequence
  Description of Artificial Sequence Synthetic primer
ctattgctgggtttgc

SEQ ID NO: 177 - DNA, 15 bp, Artificial Sequence
  Description of Artificial Sequence Synthetic primer
tattgctgggtttgc

SEQ ID NO: 178 - DNA, 15 bp, Artificial Sequence
  Description of Artificial Sequence Synthetic primer
attgctgggtttgct

SEQ ID NO: 179 - DNA, 15 bp, Artificial Sequence
  Description of Artificial Sequence Synthetic primer
ctgggtttgctgctt

SEQ ID NO: 180 - DNA, 16 bp, Artificial Sequence
  Description of Artificial Sequence Synthetic primer
ctgggtttgctgcttt

SEQ ID NO: 181 - DNA, 17 bp, Artificial Sequence
  Description of Artificial Sequence Synthetic primer
ctgggtttgctgcttta

SEQ ID NO: 182 - DNA, 16 bp, Artificial Sequence
  Description of Artificial Sequence Synthetic primer
tgggtttgctgcttta

SEQ ID NO: 183 - DNA, 18 bp, Artificial Sequence
  Description of Artificial Sequence Synthetic primer
ggtttgctgctttaattc

SEQ ID NO: 184 - DNA, 17 bp, Artificial Sequence
  Description of Artificial Sequence Synthetic primer
gtttgctgctttaattc

SEQ ID NO: 185 - DNA, 19 bp, Artificial Sequence
  Description of Artificial Sequence Synthetic primer
ctgctttaattcaaactgt

SEQ ID NO: 186 - DNA, 21 bp, Artificial Sequence
  Description of Artificial Sequence Synthetic probe
aagtgctagagcagcacccat

SEQ ID NO: 187 - DNA, 21 bp, Artificial Sequence
  Description of Artificial Sequence Synthetic probe
aaagtgctagagcagcaccca

SEQ ID NO: 188 - DNA, 25 bp, Artificial Sequence
  Description of Artificial Sequence Synthetic probe
ccaaaagtgctagagcagcacccat

SEQ ID NO: 189 - DNA, 27 bp, Artificial Sequence
  Description of Artificial Sequence Synthetic probe
caactaggtcccccaaaagtgctagag

SEQ ID NO: 190 - DNA, 23 bp, Artificial Sequence
  Description of Artificial Sequence Synthetic probe
tacactggcaactaggtccccca

SEQ ID NO: 191 - DNA, 27 bp, Artificial Sequence
  Description of Artificial Sequence Synthetic probe
tacactggcaactaggtcccccaaaag

SEQ ID NO: 192 - DNA, 25 bp, Artificial Sequence
  Description of Artificial Sequence Synthetic probe
agatacactggcaactaggtccccc

SEQ ID NO: 193 - DNA, 26 bp, Artificial Sequence
  Description of Artificial Sequence Synthetic probe
cagatacactggcaactaggtccccc

SEQ ID NO: 194 - DNA, 25 bp, Artificial Sequence
  Description of Artificial Sequence Synthetic probe
agcctcagatacactggcaactagg

SEQ ID NO: 195 - DNA, 27 bp, Artificial Sequence
  Description of Artificial Sequence Synthetic probe
agcctcagatacactggcaactaggtc

SEQ ID NO: 196 - DNA, 28 bp, Artificial Sequence
  Description of Artificial Sequence Synthetic probe
cagcctcagatacactggcaactaggtc

SEQ ID NO: 197 - DNA, 20 bp, Artificial Sequence
  Description of Artificial Sequence Synthetic probe
agcagcaatttcagccactg

SEQ ID NO: 198 - DNA, 22 bp, Artificial Sequence
  Description of Artificial Sequence Synthetic probe
tccccagcagcaatttcagcca

SEQ ID NO: 199 - DNA, 23 bp, Artificial Sequence
  Description of Artificial Sequence Synthetic probe
ctccccagcagcaatttcagcca

SEQ ID NO: 200 - DNA, 20 bp, Artificial Sequence
  Description of Artificial Sequence Synthetic probe
agcagcagcctccccagcag

SEQ ID NO: 201 - DNA, 26 bp, Artificial Sequence
  Description of Artificial Sequence Synthetic probe
accagcaattacagcatatgtttgag

SEQ ID NO: 202 - DNA, 29 bp, Artificial Sequence
  Description of Artificial Sequence Synthetic probe
agcaccagcaattacagcatatgtttgag

SEQ ID NO: 203 - DNA, 21 bp, Artificial Sequence
  Description of Artificial Sequence Synthetic probe
tgaattaaagcagcaaaccca

SEQ ID NO: 204 - DNA, 22 bp, Artificial Sequence
  Description of Artificial Sequence Synthetic probe
ttgaattaaagcagcaaaccca

SEQ ID NO: 205 - DNA, 23 bp, Artificial Sequence
  Description of Artificial Sequence Synthetic probe
agtttgaattaaagcagcaaacc

SEQ ID NO: 206 - DNA, 24 bp, Artificial Sequence
  Description of Artificial Sequence Synthetic probe
agtttgaattaaagcagcaaaccc

SEQ ID NO: 207 - DNA, 24 bp, Artificial Sequence
  Description of Artificial Sequence Synthetic probe
cagtttgaattaaagcagcaaacc

SEQ ID NO: 208 - DNA, 24 bp, Artificial Sequence
  Description of Artificial Sequence Synthetic probe
acagtttgaattaaagcagcaaac

SEQ ID NO: 209 - DNA, 26 bp, Artificial Sequence
  Description of Artificial Sequence Synthetic probe
taacagtttgaattaaagcagcaaac

SEQ ID NO: 210 - DNA, 27 bp, Artificial Sequence
  Description of Artificial Sequence Synthetic probe
taacagtttgaattaaagcagcaaacc

SEQ ID NO: 211 - DNA, 28 bp, Artificial Sequence
  Description of Artificial Sequence Synthetic probe
taacagtttgaattaaagcagcaaaccc

SEQ ID NO: 212 - DNA, 23 bp, Artificial Sequence
  Description of Artificial Sequence Synthetic probe
atcacaaagtttccactgtaggc

SEQ ID NO: 213 - DNA, 22 bp, Artificial Sequence
  Description of Artificial Sequence Synthetic probe
tcacaaagtttccactgtaggc

SEQ ID NO: 214 - DNA, 24 bp, Artificial Sequence
  Description of Artificial Sequence Synthetic probe
cctacagtggaaactttgtgatcc

SEQ ID NO: 215 - DNA, 16 bp, Artificial Sequence
  Description of Artificial Sequence Synthetic primer
agatacactggcaact

SEQ ID NO: 216 - DNA, 18 bp, Artificial Sequence
  Description of Artificial Sequence Synthetic primer
cagatacactggcaacta

SEQ ID NO: 217 - DNA, 19 bp, Artificial Sequence
  Description of Artificial Sequence Synthetic primer
tcagatacactggcaacta

SEQ ID NO: 218 - DNA, 17 bp, Artificial Sequence
  Description of Artificial Sequence Synthetic primer
gcagcctcagatacact

SEQ ID NO: 219 - DNA, 17 bp, Artificial Sequence
  Description of Artificial Sequence Synthetic primer
agcagcctcagatacac

SEQ ID NO: 220 - DNA, 17 bp, Artificial Sequence
  Description of Artificial Sequence Synthetic primer
cagcagcctcagataca

SEQ ID NO: 221 - DNA, 15 bp, Artificial Sequence
  Description of Artificial Sequence Synthetic primer
agcagcagcctcaga

SEQ ID NO: 222 - DNA, 16 bp, Artificial Sequence
  Description of Artificial Sequence Synthetic primer
agcagcagcctcagat

SEQ ID NO: 223 - DNA, 16 bp, Artificial Sequence
  Description of Artificial Sequence Synthetic primer
cagcagcagcctcaga

SEQ ID NO: 224 - DNA, 17 bp, Artificial Sequence
  Description of Artificial Sequence Synthetic primer
cagcagcagcctcagat

SEQ ID NO: 225 - DNA, 15 bp, Artificial Sequence
  Description of Artificial Sequence Synthetic primer
ccagcagcaatttca

SEQ ID NO: 226 - DNA, 16 bp, Artificial Sequence
  Description of Artificial Sequence Synthetic primer
cccagcagcaatttca

SEQ ID NO: 227 - DNA, 17 bp, Artificial Sequence
  Description of Artificial Sequence Synthetic primer
ccccagcagcaatttca

SEQ ID NO: 228 - DNA, 16 bp, Artificial Sequence
  Description of Artificial Sequence Synthetic primer
tccccagcagcaattt

SEQ ID NO: 229 - DNA, 15 bp, Artificial Sequence
  Description of Artificial Sequence Synthetic primer
ctccccagcagcaat

SEQ ID NO: 230 - DNA, 17 bp, Artificial Sequence
  Description of Artificial Sequence Synthetic primer
ctccccagcagcaattt

SEQ ID NO: 231 - DNA, 15 bp, Artificial Sequence
  Description of Artificial Sequence Synthetic primer
cctccccagcagcaa

SEQ ID NO: 232 - DNA, 16 bp, Artificial Sequence
  Description of Artificial Sequence Synthetic primer
cctccccagcagcaat

SEQ ID NO: 233 - DNA, 17 bp, Artificial Sequence
  Description of Artificial Sequence Synthetic primer
gggatgcaatttgaact

SEQ ID NO: 234 - DNA, 17 bp, Artificial Sequence
  Description of Artificial Sequence Synthetic primer
agggatgcaatttgaac

SEQ ID NO: 235 - DNA, 18 bp, Artificial Sequence
  Description of Artificial Sequence Synthetic primer
agggatgcaatttgaact

SEQ ID NO: 236 - DNA, 16 bp, Artificial Sequence
  Description of Artificial Sequence Synthetic primer
aagggatgcaatttga

SEQ ID NO: 237 - DNA, 16 bp, Artificial Sequence
  Description of Artificial Sequence Synthetic primer
gcaagggatgcaattt

SEQ ID NO: 238 - DNA, 18 bp, Artificial Sequence
  Description of Artificial Sequence Synthetic primer
cagtagcaagggatgcaa

SEQ ID NO: 239 - DNA, 16 bp, Artificial Sequence
  Description of Artificial Sequence Synthetic primer
cagctatagcctctga

SEQ ID NO: 240 - DNA, 18 bp, Artificial Sequence
  Description of Artificial Sequence Synthetic primer
agcagctatagcctctga

SEQ ID NO: 241 - DNA, 18 bp, Artificial Sequence
  Description of Artificial Sequence Synthetic primer
gaattaaagcagcaaacc

SEQ ID NO: 242 - DNA, 19 bp, Artificial Sequence
  Description of Artificial Sequence Synthetic primer
tgaattaaagcagcaaacc

SEQ ID NO: 243 - DNA, 20 bp, Artificial Sequence
  Description of Artificial Sequence Synthetic primer
gtttgaattaaagcagcaaa

SEQ ID NO: 244 - DNA, 18 bp, Artificial Sequence
  Description of Artificial Sequence Synthetic primer
acctataccctacttgag

SEQ ID NO: 245 - DNA, 17 bp, Artificial Sequence
  Description of Artificial Sequence Synthetic primer
tgatcccaatcactaaa

SEQ ID NO: 246 - DNA, 17 bp, Artificial Sequence
  Description of Artificial Sequence Synthetic primer
gtgatcccaatcactaa

SEQ ID NO: 247 - DNA, 16 bp, Artificial Sequence
  Description of Artificial Sequence Synthetic primer
agtggaaactttgtga

SEQ ID NO: 248 - DNA, 18 bp, Artificial Sequence
  Description of Artificial Sequence Synthetic primer
cagtggaaactttgtgat

SEQ ID NO: 249 - DNA, 17 bp, Artificial Sequence
  Description of Artificial Sequence Synthetic primer
ctacagtggaaactttg

SEQ ID NO: 250 - DNA, 16 bp, Artificial Sequence
  Description of Artificial Sequence Synthetic primer
gcctacagtggaaact

SEQ ID NO: 251 - DNA, 16 bp, Artificial Sequence
  Description of Artificial Sequence Synthetic primer
aggcctacagtggaaa

SEQ ID NO: 252 - DNA, 17 bp, Artificial Sequence
  Description of Artificial Sequence Synthetic primer
aggcctacagtggaaac

SEQ ID NO: 253 - DNA, 15 bp, Artificial Sequence
  Description of Artificial Sequence Synthetic primer
tgcctgattgctgat

SEQ ID NO: 254 - DNA, 16 bp, Artificial Sequence
  Description of Artificial Sequence Synthetic primer
tgcctgattgctgata

SEQ ID NO: 255 - DNA, 16 bp, Artificial Sequence
  Description of Artificial Sequence Synthetic primer
atgcctgattgctgat

SEQ ID NO: 256 - DNA, 18 bp, Artificial Sequence
  Description of Artificial Sequence Synthetic primer
caatgcagtagcaatcta

SEQ ID NO: 257 - DNA, 18 bp, Artificial Sequence
  Description of Artificial Sequence Synthetic primer
aatgcagtagcaatctat

SEQ ID NO: 258 - DNA, 19 bp, Artificial Sequence
  Description of Artificial Sequence Synthetic primer
aatgcagtagcaatctatc

SEQ ID NO: 259 - DNA, 18 bp, Artificial Sequence
  Description of Artificial Sequence Synthetic primer
tgcagtagcaatctatcc

SEQ ID NO: 260 - DNA, 18 bp, Artificial Sequence
  Description of Artificial Sequence Synthetic primer
gcagtagcaatctatcca

SEQ ID NO: 261 - DNA, 19 bp, Artificial Sequence
  Description of Artificial Sequence Synthetic primer
cagtagcaatctatccaaa

SEQ ID NO: 262 - DNA, 18 bp, Artificial Sequence
  Description of Artificial Sequence Synthetic primer
gcaatctatccaaaccaa

SEQ ID NO: 263 - DNA, 17 bp, Artificial Sequence
  Description of Artificial Sequence Synthetic primer
caatctatccaaaccaa

SEQ ID NO: 264 - DNA, 16 bp, Artificial Sequence
  Description of Artificial Sequence Synthetic primer
ctgggcaaagaggaaa

SEQ ID NO: 265 - DNA, 16 bp, Artificial Sequence
  Description of Artificial Sequence Synthetic primer
tgggcaaagaggaaaa

SEQ ID NO: 266 - DNA, 17 bp, Artificial Sequence
  Description of Artificial Sequence Synthetic primer
gggcaaagaggaaaatc

SEQ ID NO: 267 - DNA, 17 bp, Artificial Sequence
  Description of Artificial Sequence Synthetic primer
ggcaaagaggaaaatca

SEQ ID NO: 268 - DNA, 16 bp, Artificial Sequence
  Description of Artificial Sequence Synthetic primer
aaatcagcacaaacct

SEQ ID NO: 269 - DNA, 15 bp, Artificial Sequence
  Description of Artificial Sequence Synthetic primer
aatcagcacaaacct

SEQ ID NO: 270 - DNA, 16 bp, Artificial Sequence
  Description of Artificial Sequence Synthetic primer
cagcacaaacctctga

SEQ ID NO: 271 - DNA, 17 bp, Artificial Sequence
  Description of Artificial Sequence Synthetic primer
cacaaacctctgagcta

SEQ ID NO: 272 - DNA, 18 bp, Artificial Sequence
  Description of Artificial Sequence Synthetic primer
tccaggttccaaaatcag

SEQ ID NO: 273 - DNA, 20 bp, Artificial Sequence
  Description of Artificial Sequence Synthetic primer
tcattctcttcattttatcc

SEQ ID NO: 274 - DNA, 19 bp, Artificial Sequence
  Description of Artificial Sequence Synthetic primer
cattctcttcattttatcc

SEQ ID NO: 275 - DNA, 16 bp, Artificial Sequence
  Description of Artificial Sequence Synthetic primer
cctttgtcagggtgaa

SEQ ID NO: 276 - DNA, 17 bp, Artificial Sequence
  Description of Artificial Sequence Synthetic primer
ctttgtcagggtgaaat

SEQ ID NO: 277 - DNA, 16 bp, Artificial Sequence
  Description of Artificial Sequence Synthetic primer
tttgtcagggtgaaat

SEQ ID NO: 278 - DNA, 16 bp, Artificial Sequence
  Description of Artificial Sequence Synthetic primer
tcagggtgaaattcct

SEQ ID NO: 279 - DNA, 17 bp, Artificial Sequence
  Description of Artificial Sequence Synthetic primer
cagggtgaaattcctta

SEQ ID NO: 280 - DNA, 17 bp, Artificial Sequence
  Description of Artificial Sequence Synthetic primer
agggtgaaattccttac

SEQ ID NO: 281 - DNA, 17 bp, Artificial Sequence
  Description of Artificial Sequence Synthetic primer
gggtgaaattccttaca

SEQ ID NO: 282 - DNA, 24 bp, Artificial Sequence
  Description of Artificial Sequence Synthetic probe
tgattttcctctttgcccagatac

SEQ ID NO: 283 - DNA, 21 bp, Artificial Sequence
  Description of Artificial Sequence Synthetic probe
tccaggttccaaaatcaggct

SEQ ID NO: 284 - DNA, 32 bp, Artificial Sequence
  Description of Artificial Sequence Synthetic probe
aaagtattcattctcttcattttatcctcgtc

SEQ ID NO: 285 - DNA, 32 bp, Artificial Sequence
  Description of Artificial Sequence Synthetic probe
aagtattcattctcttcattttatcctcgtcg

SEQ ID NO: 286 - DNA, 31 bp, Artificial Sequence
  Description of Artificial Sequence Synthetic probe
attcattctcttcattttatcctcgtcgccc

SEQ ID NO: 287 - DNA, 29 bp, Artificial Sequence
  Description of Artificial Sequence Synthetic probe
tcattctcttcattttatcctcgtcgccc

SEQ ID NO: 288 - DNA, 29 bp, Artificial Sequence
  Description of Artificial Sequence Synthetic probe
cattctcttcattttatcctcgtcgcccc

SEQ ID NO: 289 - DNA, 28 bp, Artificial Sequence
  Description of Artificial Sequence Synthetic probe
attctcttcattttatcctcgtcgcccc

SEQ ID NO: 290 - DNA, 20 bp, Artificial Sequence
  Description of Artificial Sequence Synthetic probe
tcgtcgccccctttgtcagg

SEQ ID NO: 291 - DNA, 20 bp, Artificial Sequence
  Description of Artificial Sequence Synthetic probe
tttccccaggcagctctttc

SEQ ID NO: 292 - DNA, 17 bp, Artificial Sequence
  Description of Artificial Sequence Synthetic primer
tagctcagaggtttgtg

SEQ ID NO: 293 - DNA, 16 bp, Artificial Sequence
  Description of Artificial Sequence Synthetic primer
agctcagaggtttgtg

SEQ ID NO: 294 - DNA, 20 bp, Artificial Sequence
  Description of Artificial Sequence Synthetic primer
ggataaaatgaagagaatga

SEQ ID NO: 295 - DNA, 19 bp, Artificial Sequence
  Description of Artificial Sequence Synthetic primer
gaggataaaatgaagagaa

SEQ ID NO: 296 - DNA, 19 bp, Artificial Sequence
  Description of Artificial Sequence Synthetic primer
acgaggataaaatgaagag

SEQ ID NO: 297 - DNA, 18 bp, Artificial Sequence
  Description of Artificial Sequence Synthetic primer
cgacgaggataaaatgaa

SEQ ID NO: 298 - DNA, 17 bp, Artificial Sequence
  Description of Artificial Sequence Synthetic primer
ggcgacgaggataaaat

SEQ ID NO: 299 - DNA, 17 bp, Artificial Sequence
  Description of Artificial Sequence Synthetic primer
tttcaccctgacaaagg

SEQ ID NO: 300 - DNA, 17 bp, Artificial Sequence
  Description of Artificial Sequence Synthetic primer
ggaatttcaccctgaca

SEQ ID NO: 301 - DNA, 20 bp, Artificial Sequence
  Description of Artificial Sequence Synthetic primer
gaggataaaatgaagagaat

SEQ ID NO: 302 - DNA, 20 bp, Artificial Sequence
  Description of Artificial Sequence Synthetic primer
cgaggataaaatgaagagaa

SEQ ID NO: 303 - DNA, 19 bp, Artificial Sequence
  Description of Artificial Sequence Synthetic primer
cgacgaggataaaatgaag

SEQ ID NO: 304 - DNA, 17 bp, Artificial Sequence
  Description of Artificial Sequence Synthetic primer
gggcgacgaggataaaa

SEQ ID NO: 305 - DNA, 16 bp, Artificial Sequence
  Description of Artificial Sequence Synthetic primer
ggggcgacgaggataa

SEQ ID NO: 306 - DNA, 16 bp, Artificial Sequence
  Description of Artificial Sequence Synthetic primer
aatttcaccctgacaa

SEQ ID NO: 307 - DNA, 18 bp, Artificial Sequence
  Description of Artificial Sequence Synthetic primer
ggaatttcaccctgacaa

SEQ ID NO: 308 - DNA, 15 bp, Artificial Sequence
  Description of Artificial Sequence Synthetic primer
ggggcgacgaggata

SEQ ID NO: 309 - DNA, 17 bp, Artificial Sequence
  Description of Artificial Sequence Synthetic primer
atttcaccctgacaaag

SEQ ID NO: 310 - DNA, 18 bp, Artificial Sequence
  Description of Artificial Sequence Synthetic primer
aatttcaccctgacaaag

SEQ ID NO: 311 - DNA, 19 bp, Artificial Sequence
  Description of Artificial Sequence Synthetic primer
ggataaaatgaagagaatg

SEQ ID NO: 312 - DNA, 17 bp, Artificial Sequence
  Description of Artificial Sequence Synthetic primer
acgaggataaaatgaag

SEQ ID NO: 313 - DNA, 17 bp, Artificial Sequence
  Description of Artificial Sequence Synthetic primer
cgacgaggataaaatga

SEQ ID NO: 314 - DNA, 15 bp, Artificial Sequence
  Description of Artificial Sequence Synthetic primer
gggcgacgaggataa

SEQ ID NO: 315 - DNA, 15 bp, Artificial Sequence
  Description of Artificial Sequence Synthetic primer
tcaccctgacaaagg

SEQ ID NO: 316 - DNA, 16 bp, Artificial Sequence
  Description of Artificial Sequence Synthetic primer
tttcaccctgacaaag

SEQ ID NO: 317 - DNA, 19 bp, Artificial Sequence
  Description of Artificial Sequence Synthetic primer
cgaggataaaatgaagaga

SEQ ID NO: 318 - DNA, 16 bp, Artificial Sequence
  Description of Artificial Sequence Synthetic primer
ttcaccctgacaaagg

SEQ ID NO: 319 - DNA, 17 bp, Artificial Sequence
  Description of Artificial Sequence Synthetic primer
aatttcaccctgacaaa

SEQ ID NO: 320 - DNA, 18 bp, Artificial Sequence
  Description of Artificial Sequence Synthetic primer
atttcaccctgacaaagg

SEQ ID NO: 321 - DNA, 16 bp, Artificial Sequence
  Description of Artificial Sequence Synthetic primer
gagctgcctggggaaa

SEQ ID NO: 322 - DNA, 15 bp, Artificial Sequence
  Description of Artificial Sequence Synthetic primer
agctgcctggggaaa

SEQ ID NO: 323 - DNA, 17 bp, Artificial Sequence
  Description of Artificial Sequence Synthetic primer
gagctcatggacctttt

SEQ ID NO: 324 - DNA, 17 bp, Artificial Sequence
  Description of Artificial Sequence Synthetic primer
ggagctcatggaccttt

SEQ ID NO: 325 - DNA, 16 bp, Artificial Sequence
  Description of Artificial Sequence Synthetic primer
agctcatggacctttt

SEQ ID NO: 326 - DNA, 16 bp, Artificial Sequence
  Description of Artificial Sequence Synthetic primer
gagctcatggaccttt

SEQ ID NO: 327 - DNA, 16 bp, Artificial Sequence
  Description of Artificial Sequence Synthetic primer
ggagctcatggacctt

SEQ ID NO: 328 - DNA, 15 bp, Artificial Sequence
  Description of Artificial Sequence Synthetic primer
catggagctcatgga

SEQ ID NO: 329 - DNA, 17 bp, Artificial Sequence
  Description of Artificial Sequence Synthetic primer
cacaaacctctgagcta

SEQ ID NO: 330 - DNA, 17 bp, Artificial Sequence
  Description of Artificial Sequence Synthetic primer
tccaggttccaaaatca

SEQ ID NO: 331 - DNA, 15 bp, Artificial Sequence
  Description of Artificial Sequence Synthetic primer
ggggcgacgaggata

SEQ ID NO: 332 - DNA, 21 bp, Artificial Sequence
  Description of Artificial Sequence Synthetic probe
tccaggttccaaaatcaggct

SEQ ID NO: 333 - DNA, 23 bp, Artificial Sequence
  Description of Artificial Sequence Synthetic probe
tcttcattttatcctcgtcgccc

SEQ ID NO: 334 - DNA, 24 bp, Artificial Sequence
  Description of Artificial Sequence Synthetic probe
aagattcagcagctaccgcagccc

SEQ ID NO: 335 - DNA, 29 bp, Artificial Sequence
  Description of Artificial Sequence Synthetic oligonucleotide
gcatcgtcctttgtaacgatcaaggctgg

SEQ ID NO: 336 - DNA, 24 bp, Artificial Sequence
  Description of Artificial Sequence Synthetic oligonucleotide
gggctgcggtagctgctgaatctt

SEQ ID NO: 337 - DNA, 10 bp, Artificial Sequence
  Description of Artificial Sequence Synthetic probe
aaacacgtgc

SEQ ID NO: 338 - DNA, 10 bp, Artificial Sequence
  Description of Artificial Sequence Synthetic probe
ccttgttcca

SEQ ID NO: 339 - DNA, 10 bp, Artificial Sequence
  Description of Artificial Sequence Synthetic probe
cagggacgat

SEQ ID NO: 340 - DNA, 27 bp, Artificial Sequence
  Description of Artificial Sequence Synthetic probe
attctcttcattttatcctcgtcgccc